SEQUENCE LISTING (66 sequences)

SEQ ID NO: 1 - protein, 69 aa, Artificial Sequence [sequence omitted]

SEQ ID NO: 2 - protein, 434 aa, Helicobacter pylori
MAVQKVKNADKVQKLSDTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKYQQDNQTKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNPFRNMGMIASSTTNNGAHHHHHH

SEQ ID NO: 3 - protein, 463 aa, Artificial Sequence
  Mk6P4-CCL5c7HopQV1 Megakine
QGPPGDIVLACCFAYIARPLPRAHIKEYFYTSGKCSNPAVVFVKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCANPEKKWVREYINSLEMSHHHHHHEPEA

SEQ ID NO: 4 - protein, 464 aa, Artificial Sequence
  Mk6P4-CCL5c7HopQV2 Megakine
QGPPGDIVLACCFAYIARPLPRAHIKEYFYTSGKCSNPAVVFVTTTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCANPEKKWVREYINSLEMSHHHHHHEPEA

SEQ ID NO: 5 - protein, 465 aa, Artificial Sequence
  Mk6P4-CCL5c7HopQV3  Megakine
QGPPGDIVLACCFAYIARPLPRAHIKEYFYTSGKCSNPAVVFVTRTKTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCANPEKKWVREYINSLEMSHHHHHHEPEA

SEQ ID NO: 6 - protein, 463 aa, Artificial Sequence
  Mk6P4-CCL5c7HopQV4  Megakine
QGPPGDIVLACCFAYIARPLPRAHIKEYFYTSGKCSNPAVVFVTKTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCANPEKKWVREYINSLEMSHHHHHHEPEA

SEQ ID NO: 7 - protein, 742 aa, Artificial Sequence
  Mk6P4-CCL5c7HopQV1_Aga2p_ACP protein sequence
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCANPEKKWVREYINSLEMSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 8 - protein, 743 aa, Artificial Sequence
  Mk 6P4-CCL5c7HopQV2_Aga2p_ACP protein sequence
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVTTTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCANPEKKWVREYINSLEMSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 9 - protein, 744 aa, Artificial Sequence
  Mk6P4-CCL5c7HopQV3_Aga2p_ACP protein sequence
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVTRTKTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCANPEKKWVREYINSLEMSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 10 - protein, 742 aa, Artificial Sequence
  Mk6P4-CCL5c7HopQV4_Aga2p_ACP protein sequence
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVTKTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCANPEKKWVREYINSLEMSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 11 - protein, 821 aa, Artificial Sequence
  MbNb207cHopQ_Aga2p_ACP protein sequence
MRFPSIFTAVVFAASSALAAPANTTAEDETAQIPAEAVIGYLGLEGDSDVAALPLSDSTNNGSLSTNTTIASIAAKEEGVQLDKREAEAQVQLVESGGGLVQTKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNPFRASGGGSGGGGSGKLSDTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKYGSLRLSCAASGRTFSTAAMGWFRQAPGKERDFVAGIYWTVGSTYYADSAKGRFTISRDNAKNTVYLQMDSLKPEDTAVYYCAARRRGFTLAPTRANEYDYWGQGTQVTVSSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 12 - protein, 552 aa, Artificial Sequence
  Mk6P4-CCL5c7HopQV1 yeast secreted protein sequence
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCANPEKKWVREYINSLEMSHHHHHHEPEA

SEQ ID NO: 13 - protein, 553 aa, Artificial Sequence
  Mk6P4-CCL5c7HopQV2 yeast secreted protein sequence
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVTTTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCANPEKKWVREYINSLEMSHHHHHHEPEA

SEQ ID NO: 14 - protein, 554 aa, Artificial Sequence
  Mk6P4-CCL5c7HopQV3 yeast secreted protein sequence
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVTRTKTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCANPEKKWVREYINSLEMSHHHHHHEPEA

SEQ ID NO: 15 - protein, 552 aa, Artificial Sequence
  Mk6P4-CCL5c7HopQV4 yeast secreted protein sequence
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVTKTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCANPEKKWVREYINSLEMSHHHHHHEPEA

SEQ ID NO: 16 - protein, 482 aa, Artificial Sequence
  DsbA_Mk6P4-CCL5c7HopQV1 protein sequence
MKKIWLALAGLVLAFSASAQGPPGDIVLACCFAYIARPLPRAHIKEYFYTSGKCSNPAVVFVKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCANPEKKWVREYINSLEMSHHHHHHEPEA

SEQ ID NO: 17 - protein, 483 aa, Artificial Sequence
  DsbA_Mk6P4-CCL5c7HopQV2 protein sequence
MKKIWLALAGLVLAFSASAQGPPGDIVLACCFAYIARPLPRAHIKEYFYTSGKCSNPAVVFVTTTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCANPEKKWVREYINSLEMSHHHHHHEPEA

SEQ ID NO: 18 - protein, 484 aa, Artificial Sequence
  DsbA_Mk6P4-CCL5c7HopQV3 protein sequence
MKKIWLALAGLVLAFSASAQGPPGDIVLACCFAYIARPLPRAHIKEYFYTSGKCSNPAVVFVTRTKTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCANPEKKWVREYINSLEMSHHHHHHEPEA

SEQ ID NO: 19 - protein, 482 aa, Artificial Sequence
  DsbA_Mk6P4-CCL5c7HopQV4 protein sequence
MKKIWLALAGLVLAFSASAQGPPGDIVLACCFAYIARPLPRAHIKEYFYTSGKCSNPAVVFVTKTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCANPEKKWVREYINSLEMSHHHHHHEPEA

SEQ ID NO: 20 - protein, 542 aa, Artificial Sequence
  DsbA_MbNb207cHopQ Megabody
MKKIWLALAGLVLAFSASAQVQLVESGGGLVQTKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKYGSLRLSCAASGRTFSTAAMGWFRQAPGKERDFVAGIYWTVGSTYYADSAKGRFTISRDNAKNTVYLQMDSLKPEDTAVYYCAARRRGFTLAPTRANEYDYWGQGTQVTVSSHHHHHHEPEA

SEQ ID NO: 21 - protein, 4 aa, Artificial Sequence
  affinity tag
EPEA

SEQ ID NO: 22 - protein, 68 aa, Homo sapiens
KPVSLSYRCPCRFFESHVARANVKHLKILNTPNCALQIVARLKNNNRQVCIDPKLKWIQEYLEKALNK

SEQ ID NO: 23 - protein, 464 aa, Artificial Sequence
  MkCXCL12c7HopQ protein sequence
KPVSLSYRCPCRFFESHVARANVKHLKILNTPNCALQIVARLKTKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCIDPKLKWIQEYLEKALNKHHHHHHEPEA

SEQ ID NO: 24 - protein, 483 aa, Artificial Sequence
  DsbA-MkCXCL12c7HopQ protein sequence
MKKIWLALAGLVLAFSASAKPVSLSYRCPCRFFESHVARANVKHLKILNTPNCALQIVARLKTKTTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKNRQVCIDPKLKWIQEYLEKALNKHHHHHHEPEA

SEQ ID NO: 25 - protein, 742 aa, Artificial Sequence
  Mk6P4-CCL5c7HopQ random linkers
  misc_feature (134)..(134): Xaa can be any naturally occurring amino acid
  misc_feature (518)..(518): Xaa can be any naturally occurring amino acid
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVTXTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTXNRQVCANPEKKWVREYINSLEMSGSGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 26 - protein, 743 aa, Artificial Sequence
  Mk6P4-CCL5c7HopQ random linkers
  misc_feature (134)..(134): Xaa can be any naturally occurring amino acid
  misc_feature (518)..(519): Xaa can be any naturally occurring amino acid
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVTXTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTXXNRQVCANPEKKWVREYINSLEMSGSGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 27 - protein, 743 aa, Artificial Sequence
  Mk6P4-CCL5c7HopQ random linkers
  misc_feature (134)..(135): Xaa can be any naturally occurring amino acid
  misc_feature (519)..(519): Xaa can be any naturally occurring amino acid
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVTXXTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTXNRQVCANPEKKWVREYINSLEMSGSGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 28 - protein, 744 aa, Artificial Sequence
  Mk6P4-CCL5c7HopQ random linkers
  misc_feature (134)..(135): Xaa can be any naturally occurring amino acid
  misc_feature (519)..(520): Xaa can be any naturally occurring amino acid
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVTXXTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTXXNRQVCANPEKKWVREYINSLEMSGSGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 29 - DNA, 58 bp, Artificial Sequence
  Primer
  misc_feature (33)..(34): n is a, c, g, or t
  misc_feature (35)..(35): k is g or t
aatgtagcaatccagcagttgtttttgttaccnnkacaacttctgttattgatactac

SEQ ID NO: 30 - DNA, 61 bp, Artificial Sequence
  Primer
  misc_feature (33)..(34): n is a, c, g, or t
  misc_feature (35)..(35): k is g or t
  misc_feature (36)..(37): n is a, c, g, or t
  misc_feature (38)..(38): k is g or t
aatgtagcaatccagcagttgtttttgttaccnnknnkacaacttctgttattgatactac

SEQ ID NO: 31 - DNA, 60 bp, Artificial Sequence
  Primer
  misc_feature (36)..(36): m is a or c
  misc_feature (37)..(38): n is a, c, g, or t
cattttttttcaggatttgcacaaacctgacgattmnntgtggttacatgtgcttctaac

SEQ ID NO: 32 - DNA, 63 bp, Artificial Sequence
  Primer
  misc_feature (36)..(36): m is a or c
  misc_feature (37)..(38): n is a, c, g, or t
  misc_feature (39)..(39): m is a or c
  misc_feature (40)..(41): n is a, c, g, or t
cattttttttcaggatttgcacaaacctgacgattmnnmnntgtggttacatgtgcttctaac

SEQ ID NO: 33 - protein, 92 aa, Artificial Sequence
  SS- 6P4-CCL5
MKVSAAALAVILIATALCAPASAQGPPGDIVLACCFAYIARPLPRAHIKEYFYTSGKCSNPAVVFVTRKNRQVCANPEKKWVREYINSLEMS

SEQ ID NO: 34 - protein, 760 aa, Escherichia coli
NADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQ

SEQ ID NO: 35 - protein, 17 aa, Artificial Sequence
  cYgjk circular permutation linker peptide
ASGGGSGGGGSGGGGSG

SEQ ID NO: 36 - protein, 775 aa, Artificial Sequence
  c1YgjK scaffold protein (PDB 3W7S)
KEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKK

SEQ ID NO: 37 - protein, 775 aa, Artificial Sequence
  c2YgjK scaffold protein (PDB 3W7S)
VQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTA

SEQ ID NO: 38 - protein, 847 aa, Artificial Sequence
  Mk6P4-CCL5c1YgjKV1 Megakine
QGPPGDIVLACCFAYIARPLPRAHIKEYFYTSGKCSNPAVVFVTRGGKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGGNRQVCANPEKKWVREYINSLEMS

SEQ ID NO: 39 - protein, 845 aa, Artificial Sequence
  Mk6P4-CCL5c1YgjKV2 Megakine
QGPPGDIVLACCFAYIARPLPRAHIKEYFYTSGKCSNPAVVFVTRGKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGNRQVCANPEKKWVREYINSLEMS

SEQ ID NO: 40 - protein, 843 aa, Artificial Sequence
  Mk6P4-CCL5c1YgjKV3 Megakine
QGPPGDIVLACCFAYIARPLPRAHIKEYFYTSGKCSNPAVVFVTRKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKNRQVCANPEKKWVREYINSLEMS

SEQ ID NO: 41 - protein, 847 aa, Artificial Sequence
  Mk6P4-CCL5c2YgjKV1 Megakine
QGPPGDIVLACCFAYIARPLPRAHIKEYFYTSGKCSNPAVVFVTRGGVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAGGNRQVCANPEKKWVREYINSLEMS

SEQ ID NO: 42 - protein, 843 aa, Artificial Sequence
  Mk6P4-CCL5c2YgjKV3 Megakine
QGPPGDIVLACCFAYIARPLPRAHIKEYFYTSGKCSNPAVVFVTRVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTANRQVCANPEKKWVREYINSLEMS

SEQ ID NO: 43 - protein, 1136 aa, Artificial Sequence
  Mk6P4-CCL5c1YgjKV1_Aga2p_ACP protein sequence
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVTRGGKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGGNRQVCANPEKKWVREYINSLEMSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 44 - protein, 1134 aa, Artificial Sequence
  Mk6P4-CCL5c1YgjKV2_Aga2p_ACP protein sequence
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVTRGKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGNRQVCANPEKKWVREYINSLEMSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 45 - protein, 1132 aa, Artificial Sequence
  Mk6P4-CCL5c1YgjKV3_Aga2p_ACP protein sequence
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVTRKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAKDVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKNRQVCANPEKKWVREYINSLEMSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 46 - protein, 1136 aa, Artificial Sequence
  Mk6P4-CCL5c2YgjKV1_Aga2p_ACP protein sequence
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVTRGGVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTAGGNRQVCANPEKKWVREYINSLEMSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 47 - protein, 1132 aa, Artificial Sequence
  Mk6P4-CCL5c2YgjKV3_Aga2p_ACP protein sequence
MRFPSIFTAVVFAASSALAAPANTTAEDETAQIPAEAVIGYLGLEGDSDVAALPLSDSTNNGSLSTNTTIASIAAKEEGVQLDKREAEAQGPPGDIVLACCFAYIARPLPRAHIKEYFYTSGKCSNPAVVFVTRVQVEMTLRFATPRTSLLETKITSNKPLDLVWDGELLEKLEAKEGKPLSDKTIAGEYPDYQRKISATRDGLKVTFGKVRATWDLLTSGESEYQVHKSLPVQTEINGNRFTSKAHINGSTTLYTTYSHLLTAQEVSKEQMQIRDILARPAFYLTASQQRWEEYLKKGLTNPDATPEQTRVAVKAIETLNGNWRSPGGAVKFNTVTPSVTGRWFSGNQTWPWDTWKQAFAMAHFNPDIAKENIRAVFSWQIQPGDSVRPQDVGFVPDLIAWNLSPERGGDGGNWNERNTKPSLAAWSVMEVYNVTQDKTWVAEMYPKLVAYHDWWLRNRDHNGNGVPEYGATRDKAHNTESGEMLFTVKKGDKEETQSGLNNYARVVEKGQYDSLEIPAQVAASWESGRDDAAVFGFIDKEQLDKYVANGGKRSDWTVKFAENRSQDGTLLGYSLLQESVDQASYMYSDNHYLAEMATILGKPEEAKRYRQLAQQLADYINTCMFDPTTQFYYDVRIEDKPLANGCAGKPIVERGKGPEGWSPLFNGAATQANADAVVKVMLDPKEFNTFVPLGTAALTNPAFGADIYWRGRVWVDQFWFGLKGMERYGYRDDALKLADTFFRHAKGLTADGPIQENYNPLTGAQQGAPNFSWSAAHLYMLYNDFFRKQASGGGSGGGGSGGGGSGNADNYKNVINRTGAPQYMKDYDYDDHQRFNPFFDLGAWHGHLLPDGPNTMGGFPGVALLTEEYINFMASNFDRLTVWQDGKKVDFTLEAYSIPGALVQKLTANRQVCANPEKKWVREYINSLEMSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 48 - protein, 153 aa, Homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 49 - protein, 539 aa, Artificial Sequence
  MkIL-1 c7HopQV1 Megakine
APVRSLNCTLRDSQQKSLVMSGPYELKALHLQGQDMEQQVVFSMSFVQGEESNDKIPVALGLKEKNLYLSCVLGGKTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKGGPTLQLESVDPKNYPKKKMEKRFVFNKIEINNKLEFESAQFPNWYISTSQAENMPVFLGGTKGGQDITDFTMQFVSS

SEQ ID NO: 50 - protein, 537 aa, Artificial Sequence
  MkIL-1 c7HopQV2 Megakine
APVRSLNCTLRDSQQKSLVMSGPYELKALHLQGQDMEQQVVFSMSFVQGEESNDKIPVALGLKEKNLYLSCVLGKTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKGPTLQLESVDPKNYPKKKMEKRFVFNKIEINNKLEFESAQFPNWYISTSQAENMPVFLGGTKGGQDITDFTMQFVSS

SEQ ID NO: 51 - protein, 535 aa, Artificial Sequence
  MkIL-1 c7HopQV3 Megakine
APVRSLNCTLRDSQQKSLVMSGPYELKALHLQGQDMEQQVVFSMSFVQGEESNDKIPVALGLKEKNLYLSCVLKTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKPTLQLESVDPKNYPKKKMEKRFVFNKIEINNKLEFESAQFPNWYISTSQAENMPVFLGGTKGGQDITDFTMQFVSS

SEQ ID NO: 52 - protein, 828 aa, Artificial Sequence
  MkIL-1 c7HopQV1_Aga2p_ACP protein sequence
MRFPSIFTAVVFAASSALAAPANTTAEDETAQIPAEAVIGYLGLEGDSDVAALPLSDSTNNGSLSTNTTIASIAAKEEGVQLDKREAEAAPVRSLNCTLRDSQQKSLVMSGPYELKALHLQGQDMEQQVVFSMSFVQGEESNDKIPVALGLKEKNLYLSCVLGGKTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKGGPTLQLESVDPKNYPKKKMEKRFVFNKIEINNKLEFESAQFPNWYISTSQAENMPVFLGGTKGGQDITDFTMQFVSSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 53 - protein, 826 aa, Artificial Sequence
  MkIL-1 c7HopQV2_Aga2p_ACP protein sequence
MRFPSIFTAVVFAASSALAAPANTTAEDETAQIPAEAVIGYLGLEGDSDVAALPLSDSTNNGSLSTNTTIASIAAKEEGVQLDKREAEAAPVRSLNCTLRDSQQKSLVMSGPYELKALHLQGQDMEQQVVFSMSFVQGEESNDKIPVALGLKEKNLYLSCVLGKTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKGPTLQLESVDPKNYPKKKMEKRFVFNKIEINNKLEFESAQFPNWYISTSQAENMPVFLGGTKGGQDITDFTMQFVSSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 54 - protein, 824 aa, Artificial Sequence
  MkIL-1 c7HopQV3_Aga2p_ACP protein sequence
MRFPSIFTAVVFAASSALAAPANTTAEDETAQIPAEAVIGYLGLEGDSDVAALPLSDSTNNGSLSTNTTIASIAAKEEGVQLDKREAEAAPVRSLNCTLRDSQQKSLVMSGPYELKALHLQGQDMEQQVVFSMSFVQGEESNDKIPVALGLKEKNLYLSCVLKTTSVIDTTNDAQNLLTQAQTIVNTLKDYCPILIAKSSSSNGGTNNANTPSWQTAGGGKNSCATFGAEFSAASDMINNAQKIVQETQQLSANQPKNITQPHNLNLNSPSSLTALAQKMLKNAQSQAEILKLANQVESDFNKLSSGHLKDYIGKCDASAISSANMTMQNQKNNWGNGCAGVEETQSLLKTSAADFNNQTPQINQAQNLANTLIQELGNNTYEQLSRLLTNDNGTNSKTSAQAINQAVNNLNERAKTLAGGTTNSPAYQATLLALRSVLGLWNSMGYAVICGGYTKSPGENNQKDFHYTDENGNGTTINCGGSTNSNGTHSYNGTNTLKADKNVSLSIEQYEKIHEAYQILSKALKQAGLAPLNSKGEKLEAHVTTSKPTLQLESVDPKNYPKKKMEKRFVFNKIEINNKLEFESAQFPNWYISTSQAENMPVFLGGTKGGQDITDFTMQFVSSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 55 - protein, 442 aa, Artificial Sequence
  IL-1 _Aga2p_ACP protein sequence
MRFPSIFTAVVFAASSALAAPANTTAEDETAQIPAEAVIGYLGLEGDSDVAALPLSDSTNNGSLSTNTTIASIAAKEEGVQLDKREAEAAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSSLGGGSGGGGSGGGGSGGGGSGGGGSGGGGSGGGGSQELTTICEQIPSPTLESTPYSLSTTTILANGKAMQGVFEYYKSVTFVSNCGSHPSTTSKGSPINTQYVFKDNSSTSMSTIEERVKKIIGEQLGVKQEEVTNNASFVEDLGADSLDTVELVMALEEEFDTEIPDEEAEKITTVQAAIDYINGHQASEQKLISEEDL

SEQ ID NO: 56 - protein, 11 aa, Artificial Sequence
  Nanoluciferase subunit I
VTGYRLFEEIL

SEQ ID NO: 57 - protein, 15 aa, Artificial Sequence
  15 Gly/Ser linker
GSSGGGGSGGGGSSG

SEQ ID NO: 58 - protein, 4 aa, Artificial Sequence
  G/S linker
GGSG

SEQ ID NO: 59 - protein, 6 aa, Artificial Sequence
  Linker
  misc_feature (4)..(4): Xaa can be any naturally occurring amino acid
FVTXTT

SEQ ID NO: 60 - protein, 7 aa, Artificial Sequence
  Linker
  misc_feature (4)..(4): Xaa can be any naturally occurring amino acid
VTTXNRQ

SEQ ID NO: 61 - protein, 8 aa, Artificial Sequence
  Linker
  misc_feature (4)..(5): Xaa can be any naturally occurring amino acid
VTTXXNRQ

SEQ ID NO: 62 - protein, 7 aa, Artificial Sequence
  Linker
  misc_feature (4)..(5): Xaa can be any naturally occurring amino acid
FVTXXTT

SEQ ID NO: 63 - protein, 11 aa, Artificial Sequence
  Linker
AVVFVKTTTSV

SEQ ID NO: 64 - protein, 12 aa, Artificial Sequence
  Linker
AVVFVTTTTTSV

SEQ ID NO: 65 - protein, 14 aa, Artificial Sequence
  Linker
AVVFVTRTKTTTSV

SEQ ID NO: 66 - protein, 12 aa, Artificial Sequence
  Linker
AVVFVTKTTTSV

(19) United States
(12) Patent Application Publication
(10) Pub. No.: US 2022/0064245 A1
(43) Pub. Date: Mar. 3, 2022

(54) FUSION PROTEINS COMPRISING A CYTOKINE AND SCAFFOLD PROTEIN

(71) Applicants: VIB VZW, Gent (BE); VRIJE UNIVERSITEIT BRUSSEL, Brussel (BE); LUXEMBOURG INSTITUTE OF HEALTH, Luxembourg (LU)

(72) Inventors: Jan Steyaert, Beersel (BE); Els Pardon, Wezemaal (BE); Alexandre Wohlkönig, Brussel (BE); Valentina Kalichuk, Brussel (BE); Wim Vranken, Brussel (BE); Tomasz Uchanski, Libramont-Chevigny (BE); Andy Chevigné, Saint Léger (BE); Martyna Szpakowska, Luxembourg (LU)

(21) Appl. No.: 17/415,355

(22) PCT Filed: Dec. 20, 2019
(86) PCT No.: PCT/EP2019/086696

§ 371 Date: Jun. 17, 2021

(30) Foreign Application Priority Data
Dec. 21, 2018 (EP) 18215463.3

(51) Int. Cl. C07K 14/52

(52) U.S. Cl. CPC C07K 14/523; C07K 2319/60; C07K 2319/35

(57) ABSTRACT

The present invention relates to the field of structural biology. More specifically, the present invention relates to novel fusion proteins, their uses and methods in three-dimensional structural analysis of macromolecules, such as X-ray crystallography and high-resolution Cryo-EM, and their use in structure-based drug design and screening. Even more specifically, the invention relates to a functional fusion protein of a cytokine and a scaffold protein wherein the scaffold is a folded protein that interrupts the topology of the cytokine to form a rigid fusion protein that retains its receptor binding and activation capacity. More specifically, chemokine- and interleukin-based functional fusion proteins, and their production and uses, are disclosed herein.

### CROSS-REFERENCE TO RELATED APPLICATIONS

This application is a national phase entry under 35 U.S.C. § 371 of International Patent Application PCT/EP2019/086696, filed Dec. 20, 2019, designating the United States of America and published in English as International Patent Publication WO 2020/127983 on Jun. 25, 2020, which claims the benefit under Article 8 of the Patent Cooperation Treaty to European Patent Application Serial No. 18215463.3, filed Dec. 21, 2018, the entireties of which are hereby incorporated by reference.

### FIELD OF THE INVENTION

The present invention relates to the field of structural biology. More specifically, the present invention relates to novel fusion proteins, their uses and methods in three-dimensional structural analysis of macromolecules, such as X-ray crystallography and high-resolution Cryo-EM, and their use in structure-based drug design and screening. Even more specifically, the invention relates to a functional fusion protein of a cytokine and a scaffold protein wherein the scaffold is a folded protein that interrupts the topology of the cytokine to form a rigid fusion protein that retains its receptor binding and activation capacity. More specifically, chemokine- and interleukin-based functional fusion proteins, and their production and uses, are disclosed herein.

### BACKGROUND

The 3D-structural analysis of many proteins and complexes in certain conformational states remains difficult. Macromolecular X-ray crystallography intrinsically holds several disadvantages, such as the prerequisite for high quality purified protein, the relatively large amounts of protein that are required, and the preparation of diffraction quality crystals. The application of crystallization chaperones in the form of antibody fragments or other proteins has been proven to facilitate obtaining well-ordered crystals by minimizing the conformational heterogeneity of the target. Additionally, the chaperone can provide initial model-based phasing information (Koide, 2009). Still, single particle electron cryomicroscopy (cryo-EM) has recently developed into an alternative and versatile technique for structural analysis of macromolecular complexes at atomic resolution (Nogales, 2016). Although instrumentation and methods for data analysis improve steadily, the highest achievable resolution of the 3D reconstruction is mostly dependent on the homogeneity of a given sample, and the ability to iteratively refine the orientation parameters of each individual particle to high accuracy. Preferred particle orientation due to surface properties of the macromolecules that cause specific regions to preferentially adhere to the air-water interface or substrate support represent a recurring issue in cryo-EM. So also in this aspect, we are still missing tools such as next generation chaperones to overcome these hurdles.

Cytokines are a class of small proteins (5-20 kDa) that act as cell signaling molecules at picomolar or nanomolar concentrations to regulate inflammation and modulate cellular activities such as migration, growth, survival, and differentiation. Cytokines are an exceptionally large and diverse group of pro- or anti-inflammatory factors that are grouped into families based upon their structural homology or that of their receptors. Cytokines may include chemokines, interferons, interleukins, lymphokines, tumor necrosis factors, hormones or growth factors. Interleukins (ILs) form a group of cytokines with complex immunomodulatory functions including cell proliferation, maturation, migration and adhesion, playing an important role in immune cell differentiation and activation. ILs can also have pro- and anti-inflammatory effects, and are under constant pressure to evolve due to continual competition between the host's immune system and infecting organisms; as such, ILs have undergone significant evolution, which has resulted in little amino acid conservation between orthologous proteins, complicating the gene family organisation. Though, crystallographic data and the identification of common structural motifs have led to a classification into four major groups including the genes encoding the IL1-like cytokines, the class I helical cytokines (IL4-like, γ-chain and IL6/12-like), the class II helical cytokines (IL10-like and IL28-like) and the IL17-like cytokines, being structurally unrelated to other IL subfamily, and with IL17F constituting a cysteine-knot fold.

Chemokines are a group of secreted small globular proteins within the cytokine family whose generic function is to induce cell migration. The binding of a cytokine or chemokine ligand to its cognate receptor results in the activation of the receptor, which in turn triggers a cascade of signaling events that regulate various cellular functions such as cell adhesion, phagocytosis, cytokine secretion, cell activation, cell proliferation, cell survival and cell death, apoptosis, angiogenesis, and proliferation.

Chemokines accumulate in gradients on cell surfaces and the extracellular matrix and are interpreted as directional signals by chemokine receptors on migrating cells. Most chemokine receptors are seven-transmembrane (7TM) G-protein coupled receptors (GPCRs) that activate Gαi-dependent intracellular pathways in response to chemokine binding. Some chemokine receptors transport or scavenge chemokines via other mechanisms and are therefore referred to as atypical chemokine receptors (ACKRs). These “chemotactic cytokines” are involved in leukocyte chemoattraction and trafficking of immune cells to locations throughout the body. The chemokine system is involved in many disease areas, such as inflammatory pathologies such as asthma, atherosclerosis, and rheumatoid arthritis and also auto-immune diseases. Cytokines and chemokines play an important role in mediating neuroinflammation and neurodegeneration in various kinds of inflammatory neurodegenerative diseases including bacterial meningitis, brain abscesses, Lyme neuroborreliosis, and HIV encephalitis (for a review see Ramesh et al., 2013). Therefore, the understanding of the system is crucial for appropriate therapeutic target selection and attributing specificity.

Chemokines are small proteins of about 7-12 kDa, classified in four subfamilies based on a characteristic pattern of cysteine residues close to the amino terminus of the mature ligand (CC, CXC, CX3C, and C). All chemokines show a homologous tertiary structure and interact in different oligomerization states with cell surface glycosaminoglycans (GAGs) as well as with chemokine receptors. There are about 45 human chemokines and 22 chemokine receptors known today, with the chemokines within the same subfamily often binding multiple receptors of the same class. Although chemokines appear in dimeric form, it is their monomeric form that binds to activate the chemokine receptors. The two-site model of receptor binding and activation involves the N-terminus of the chemokine being essential in receptor activation, and the chemokine core domain mediating receptor binding. Natural chemokines have different receptor specificity, and variants of known chemokines were shown to dictate different conformational states of their receptors, leading to different signaling and responses. Some chemokines thereby act as agonists of a given receptor, while others can act as antagonists or inverse agonists. To fully understand this recognition and activation mechanism, high-resolution structures of chemokines or variants in complex with intact receptors are required. For instance, structural investigation of several CCL5 (or RANTES) variants known as agonist and antagonist are being investigated in their potential in protection to HIV as a microbicide (Kufareva et al., 2015). Several structures of chemokines are known, and for the more tractable GPCRs recapitulated as soluble complexes, structures have been resolved (β2-adrenergic receptor, rhodopsin). Structural insights in chemokine/receptor complexes and interactions are however still limited and form a challenge due to the conformational flexibility of the receptors as transmembrane proteins. Crystal structures have been determined for chemokine receptors CXCR4 and CCR5 GPCRs in complex with small molecules and, for CCR5 in complex with the antagonist chemokine variant 5P7-CCL5, for CXCR4 in complex with the viral antagonist chemokine vMIP-II, as well as for viral receptor US28 in complex with human CX3CL1. Moreover, for available crystal structures of G-protein- and β-arrestin complexed GPCRs no clear pronounced conformational difference in the receptors was seen when compared with each other, indicating that novel insights in the ligand-receptor pairs are essential in assessing their druggability (Proudfoot et al. 2015). Alternative methods to reveal structural information such as radiolytic footprinting, disulfide trapping, and mutagenesis are applied, for instance to map the structures of ACKR3:CXCL12 and ACKR3:small-molecule complexes (Gustaysson et al., 2017).

Such technologies provide for dynamic regions that proved unresolvable by X-ray crystallography in homologous receptors, integrated with molecular modelling to produce complete and cohesive experimentally driven models for expanding existing knowledge of the architecture of receptor:chemokine and receptor:small-molecule complexes. However, to explore novel routes and discover new mechanisms of ligand induced conformational changes in GPCRs, as well as other chemokine, interleukin or overall ‘cytokine receptors’, a generic prototype chaperone to facilitate X-ray crystallography or cryo-EM analysis of such complexes with their ligands, ligand analogues or variants is needed.

### SUMMARY OF THE INVENTION

The present application relates to the design and generation of novel functional fusion proteins and uses thereof, such as their role as next generation chaperones in structural analysis. The fusion proteins as described herein are based on the finding that cytokine ligands can be enlarged into rigid fusion proteins to facilitate the structural analysis of ligand/receptor complexes in certain conformational states. In fact, the disclosure provides for a fusion protein based on the given that superfamilies of cytokines share sequence similarity and exhibit structural homology and some promiscuity in their reciprocal receptor systems, although they do not exhibit functional similarity. Since cytokines are grouped according to their structure, one can start from the similarities in structural elements within a subgroup of cytokines to design the generic fusion scheme. Interleukins are a subgroup of cytokines, of which for instance the IL-1 superfamily adopts a conserved signature β-trefoil fold comprised of anti-parallel β-strands that are arranged in a three-fold symmetric pattern, with a conserved β-barrel hydrophobic core motif with significant flexibility in the loop regions. Chemokines are another subgroup of cytokines that show a very similar basic tertiary structure, with a chemokine core domain comprising a β-sheet with at least 3 β-strands. Structural conservation of said subfamilies position cytokinins ideally to offer a generic approach and prototype as next-generation chaperones in structural analysis of ligand/receptor complexes. Since the tertiary structure is homologous among these subfamilies, such as the ‘IL-1 receptor type interleukins’ or ‘IL-1 family’, as used interchangeably herein, and chemokines, with a conserved core comprising secondary β-structures (β-sheet or -barrel) providing interconnections of their β-strands via exposed turns or loops, the physical position in their core domains that is exposed and accessible for fusion with a scaffold protein can be generally applied as an example to form a ligand-integrated chaperone for structural analysis of β-strand domain-containing cytokines within cytokine/receptor complexes. Interleukin-1 or chemokine ligands were used to build a rigid larger ligand, known as a MegaKine™, and surprisingly, the enlarged ligand fusion protein retained its receptor binding and activation capacity. These novel functional fusion proteins provide for new routes to trap receptors such as GPCRs in different conformational states and facilitate their structural analysis. The novel fusion formed by rigidly inserting a scaffold protein within the cytokine core domain in such a way that it interrupts the topology of the cytokine its core domain without interfering with its folding or functionality, allows for new approaches in structure-based drug discovery. The resulting functional fusion protein is obtained via expression of a genetic fusion between said cytokine (as demonstrated for the chemokines and IL-1β) and the scaffold protein, designed so that the scaffold, or fragments thereof, inserts within the topology of the cytokine core domain. It is surprisingly shown that the resulting novel fusion proteins are characterized by a high rigidity at their fusion regions and surprisingly retain their typical fold and functionality, i.e. they retain binding affinity, and moreover showed activation capacity upon binding of the cytokine receptor. In fact, the genetic fusions made between the cytokine its conserved core domain, at an accessible site of an exposed β-turn, and the scaffold protein, are selected by the skilled person as not to disturb or alter the receptor binding. The present invention thus provides a novel and unique type of functional fusion proteins by having immaculately selected sites in exposed β-turn or -loop within the cytokine conserved core domain, such as the chemokine core domain, i.e. between β-strand β2 and β-strand β3, or the IL-1 β-barrel core motif, i.e. between β-strand β6 and β-strand β7, to allow rigid non-flexible fusions with a folded scaffold protein, which are not straightforward to design. The fusion proteins thereby provide for a novel tool to facilitate high-resolution cryo-EM and X-ray crystallography structural analysis of chemokine ligand/receptor complexes by adding mass and supplying structural features. So the design and generation of these next-generation chaperones for the structural analysis of any possible complex of cytokine, especially chemokine or variant ligand thereof, or interleukin, IL-1 or variant thereof, with its receptor allows for an enlarged ligand which adds mass and/or adds defined features to the complex of interest to obtain high resolution structures without altering conformational states. In fact, the fusion proteins are therefore advantageous as a tool in structural analysis, but also in structure-based drug design and screening, and become an added value for discovery and development of novel biologicals and small molecule agents.

The first aspect of the invention relates to a novel fusion protein comprising a functional cytokine, which is connected to a scaffold or fusion partnering protein, wherein said scaffold protein is a folded protein of at least 50 amino acids and is coupled to the cytokine at one or more amino acid positions that are accessible, hence exposed at the surface, of said cytokine, resulting in an interruption of the topology of said cytokine. Said fusion protein is further characterized in that it is functional, i.e. it retains its cytokine functionality as compared to the cytokine ligand that is not fused to said scaffold protein. Another embodiment discloses the fusion protein of the invention, wherein the fusion of scaffold protein and cytokine protein results in an interrupted primary topology of the cytokine, allowing to retain the folding and typical tertiary structure of cytokine protein, as compared to the folding of the cytokine ligand that is not fused to another protein. More specifically, the accessible amino acid positions are present in exposed regions of a beta turn (β-turn) or -loop, which interconnects the β-strand structures of the conserved cytokines.

In a particular embodiment of the invention, the fusions can be direct fusions, or fusions made by a linker or linker peptide, said fusion sites being immaculately designed to result in a rigid, non-flexible fusion protein. Preferably, the linker comprises five, four, three, or more preferably two, and even more preferably one amino acid residue, or is a direct fusion (no linker).

Said fusion protein with a scaffold protein coupled to the cytokine or chemokine core domain at one or more accessible or exposed sites at the surface of the chemokine core domain is further characterized in that said accessible or exposed sites are not in the region responsible or involved in receptor binding and receptor activating, as to retain its cytokine functionality in binding and/or activating the receptor.

One embodiment of the invention relates to a novel fusion protein wherein said cytokine is a functional chemokine, which is connected to a scaffold or fusion partnering protein, wherein said scaffold protein is coupled to the core domain of the chemokine at one or more amino acid positions that are accessible, hence exposed at the surface, of said domain, resulting in an interruption of the topology of said chemokine. Said fusion protein is further characterized in that it retains its chemokine functionality as compared to the chemokine ligand that is not fused to said scaffold protein. Another embodiment discloses the fusion protein of the invention, wherein the fusion of scaffold protein and chemokine core domain results in an interrupted primary topology of the chemokine core domain, allowing to retain the folding and typical tertiary structure of said chemokine core domain, as compared to the folding of the chemokine ligand that is not fused to another protein. In one embodiment, said fusion protein comprises a chemokine core domain with an N-terminal loop, a β sheet containing 3 β-strands, and a C-terminal helix. In a particular embodiment, the exposed region in said chemokine core domain of the fusion protein specifically concerns the β-turn that connects β-strand β2 and β-strand β3. So, the scaffold protein is inserted within the core domain at the accessible sites present in the β-turn between those 2 β-strands.

An alternative embodiment relates to the fusion protein wherein said cytokine is an interleukin, preferably an ‘IL-1 family’ interleukin, and wherein said scaffold protein interrupts the topology of the interleukin β-barrel core motif at one or more accessible sites in an exposed β-turn of said β-barrel core motif. In a particular embodiment, the exposed region in said conserved β-barrel core motif of the fusion protein specifically concerns the β-turn that connects β-strand β6 and β-strand β7. So, the scaffold protein is inserted within the core motif at the accessible sites present in the β-turn between those 2 β-strands.

In another embodiment of the invention, the scaffold protein used to generate the fusion protein is a circularly permutated protein, more specifically, the circular permutation can be made between the N- and C-terminus of said scaffold protein. In certain embodiments, the circularly permutated scaffold protein is cleaved at another accessible site of said scaffold protein, to provide a site for fusion to the accessible site(s) of the chemokine core domain. Another embodiment of the invention relates to fusion proteins wherein the total molecular mass of the scaffold protein is at least 30 kDa.

A further aspect of the invention relates to a nucleic acid molecule encoding any the fusion protein as described herein. Alternatively, in one embodiment, a chimeric gene is provided with at least a promoter, said nucleic acid molecule encoding the fusion protein, and a 3′ end region containing a transcription termination signal. Another embodiment relates to an expression cassette encoding said fusion protein or comprising the nucleic acid molecule encoding said fusion protein. Further embodiments relate to vectors comprising said nucleic acid molecule encoding the fusion protein of the invention. In particular embodiments, said vector is suited for expression in E. coli, or alternative hosts as presented herein, and for yeast, phage, bacteria or viral (surface) display. In another embodiment, a host cell comprising the fusion protein of the invention is disclosed. Alternatively, a host cell wherein said fusion protein and the cytokine or chemokine receptor, which is capable of binding the cytokine part of the fusion protein, are co-expressed.

Another aspect of the invention relates to a complex comprising said fusion protein, and the cytokine receptor. More specifically the complex comprising the chemokine or interleukin receptor, which is capable of binding the cytokine part of the fusion protein, or in particular the chemokine or interleukin part of the fusion protein, and said fusion protein, wherein said receptor protein is specifically bound to said fusion protein. More particular, wherein said receptor protein is bound to the cytokine part or alternatively to the chemokine or interleukin part of said the fusion protein, even more particular, to the known receptor binding region(s) of the fusion protein. In a certain embodiment, the complex as described herein comprises an activated receptor, wherein said receptor was activated upon binding with the fusion protein at its cytokine receptor-binding region or specifically at its chemokine or interleukin receptor-binding region.

Another aspect of the invention relates to a method for determining the 3-dimensional structure of a cytokine receptor complex, comprising the steps of:
(i) Providing the fusion protein of the present invention, and the cytokine receptor (such as a chemokine/interleukin receptor) to form a complex, wherein said receptor protein is specifically bound to the cytokine of the fusion protein, (such as respectively, the chemokine or interleukin of the fusion protein), or alternatively, providing the complex of the current invention;
(ii) and display said mix or complex in suitable conditions, for structural analysis,
wherein the 3D structure of said ligand/receptor complex is determined at high-resolution through said structural analysis.

Another aspect relates to a method for producing the functional fusion protein as described herein, comprising the steps of:
a. selecting a cytokine superfamily, such as chemokine or interleukin-1-like ligand, and a scaffold protein of which the 3-D structure reveals a folded protein of at least 10 kDa, wherein the cytokine has accessible sites in exposed β-loops or -turns for interruption of the amino acid sequence without interrupting the primary topology of the conserved cytokine core domain,
b. designing a genetic fusion construct wherein the nucleic acid sequence is designed to encode a protein sequence wherein:
   (i) the protein sequence of the cytokine ligand is interrupted at the amino acid position corresponding to the site between two β-strands of its conserved core domain structure, which is a β-loop or -turn exposed to the surface,
   (ii) the most N-terminal interrupted amino acid site of the cytokine (C-terminally of the most N-terminal β-strand is fused to the most N-terminally interrupted site of the scaffold protein, and the most C-terminal interrupted site of the cytokine (N-terminally of the most C-terminal β-strand) is fused to the most C-terminally interrupted site of the scaffold protein,
c. introducing said genetic fusion construct in an expression system to obtain a fusion protein wherein said chemokine is fused at two or more sites of its core domain to the scaffold protein.

An alternative embodiment discloses the method for producing a fusion protein as described herein, comprising the steps of:
a. selecting a chemokine and a folded scaffold protein with accessible loops or turns in their tertiary structure, which are interrupted to create a protein sequence of the fusion protein without interruption of primary topology of the chemokine or of the scaffold protein,
b. designing a genetic fusion construct wherein the nucleic acid sequence is designed to encode a protein sequence wherein:
   (i) the protein sequence of the chemokine is interrupted at an amino acid corresponding to an accessible site between the β-strand β2 and β-strand β3 of the core domain,
   (ii) the scaffold protein is at least 10 kDa and is at its N- and C-terminal ends fused to obtain a circularly permutated scaffold protein,
   (iii) the circularly permutated scaffold protein of ii) is further interrupted in its amino acid sequence at an accessible site corresponding to an exposed β-loop or -turn, which is not containing the amino acids that were fused in step ii)
   (iv) the interrupted site of the chemokine C-terminally of β-strand β2 is fused to the most N-terminally interrupted amino acid residue, i.e. the N-terminus of the circularly permutated scaffold protein, and the interrupted site of the chemokine N-terminally of β-strand β3 is fused to the most C-terminally interrupted amino acid residue, i.e. the C-terminus of the circularly permutated scaffold protein,
c. introducing said genetic fusion construct in an expression system to obtain a fusion protein wherein said chemokine is fused at two or more sites of its core domain to the circularly permutated scaffold protein.

Another aspect relates to the use of the fusion protein of the present invention or to the use of the nucleic acid molecule, the vectors, the host cell, or the complex, for structural analysis of a cytokine ligand/receptor protein. In particular, the use of the fusion protein wherein said cytokine receptor (or chemokine/interleukin/ . . . -receptor) protein is a protein bound to said fusion protein. Specifically, an embodiment relates to the use of the fusion protein in structural analysis comprising single particle cryo-EM or comprising crystallography.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIGS. 1A and 1B. Flexible fusion proteins compared to rigid chemokine chimeric proteins.

(FIG. 1A) Flexible fusions or linkers at the N- or C-terminal end of a chemokine domain and a scaffold protein using only one direct fusion or linker. (FIG. 1B) Rigid fusions of a chemokine domain and a scaffold protein, wherein the chemokine domain is fused with the scaffold protein via at least two direct fusions or linkers that connect chemokine domain to scaffold.

FIG. 2. Engineering principles of a chemokine fusion protein built from a circularly permutated variant of a scaffold protein that is inserted into the β-turn connecting β-strands β2 and β3 of a chemokine.

This scheme shows how a chemokine can be grafted onto a large scaffold protein via two peptide bonds or two short linkers that connect the chemokine domain to the scaffold. Scissors indicate which exposed turns have to be cut in the chemokine and the scaffold. Dashed lines indicate how the remaining parts of the chemokine and the scaffold have to be concatenated by use of peptide bonds or short peptide linkers to build the chemokine chimeric protein.

FIGS. 3A-3B. Model 1 of a 50 kD 6P4-CCL5 fusion protein built from a circularly permutated variant of HopQ inserted into the β-turn connecting β-strands β2 and β3 of the 6P4-CCL5 chemokine.

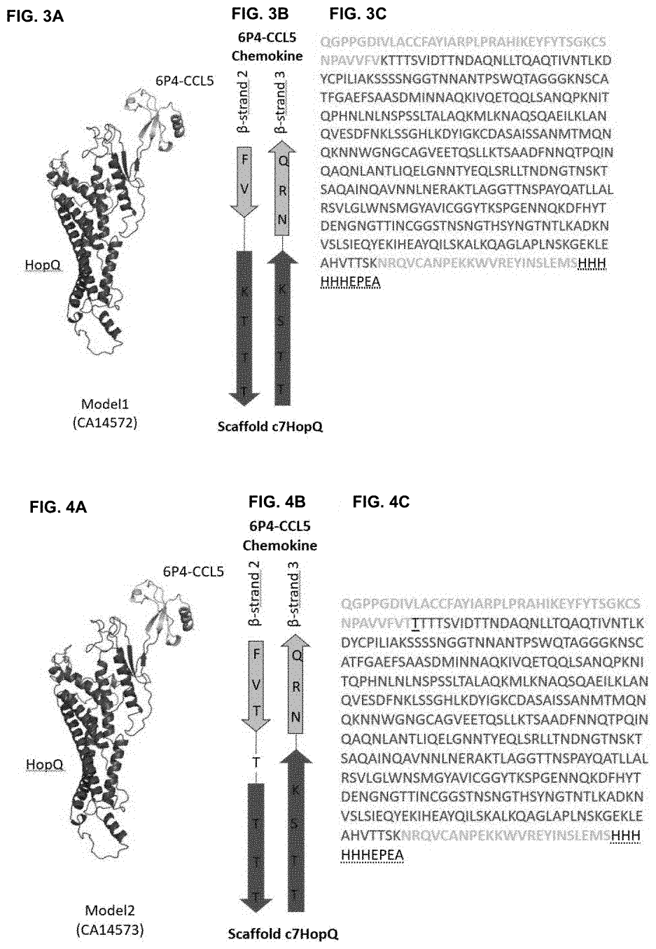

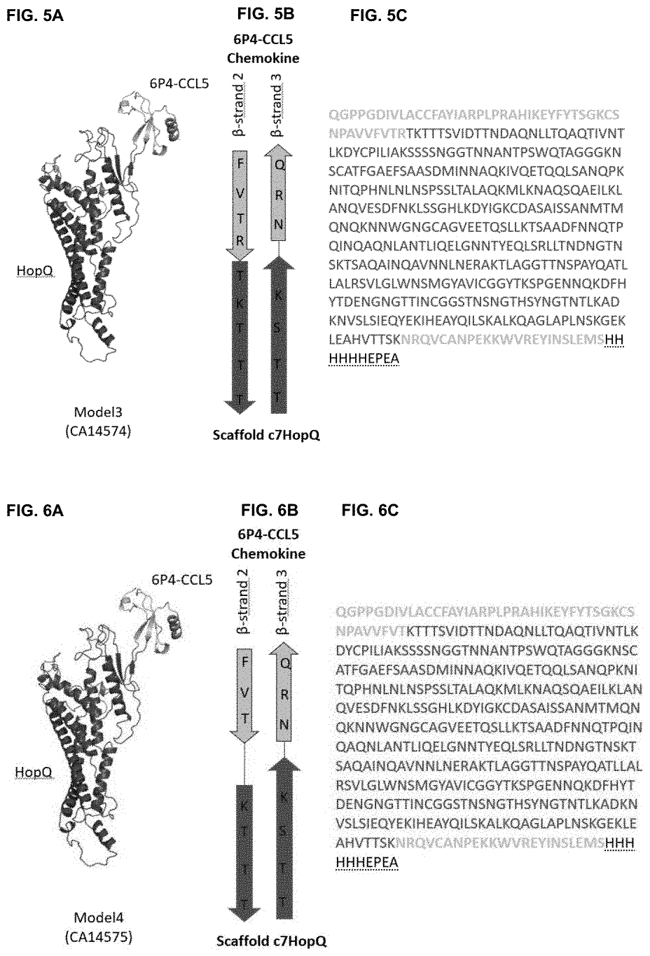

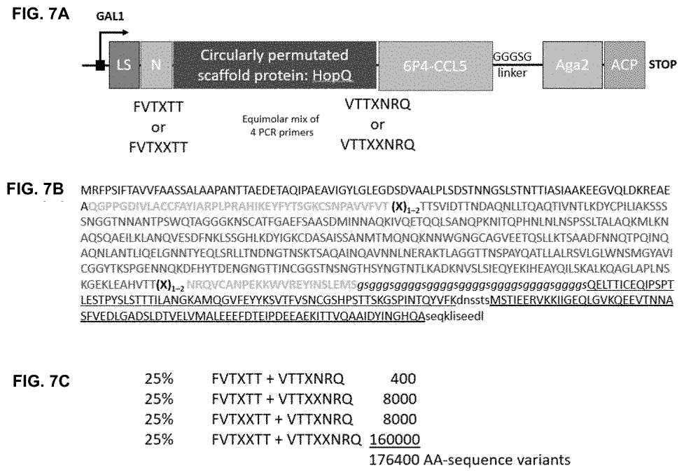

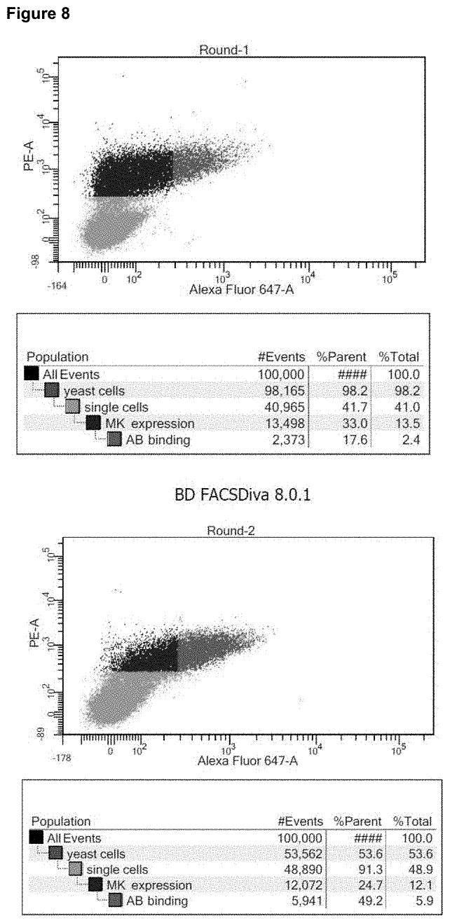

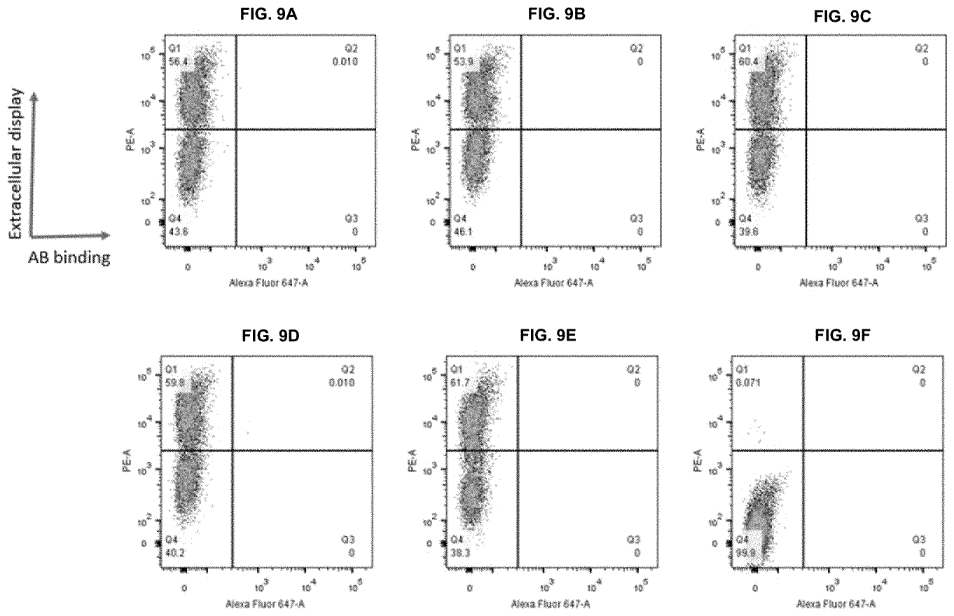

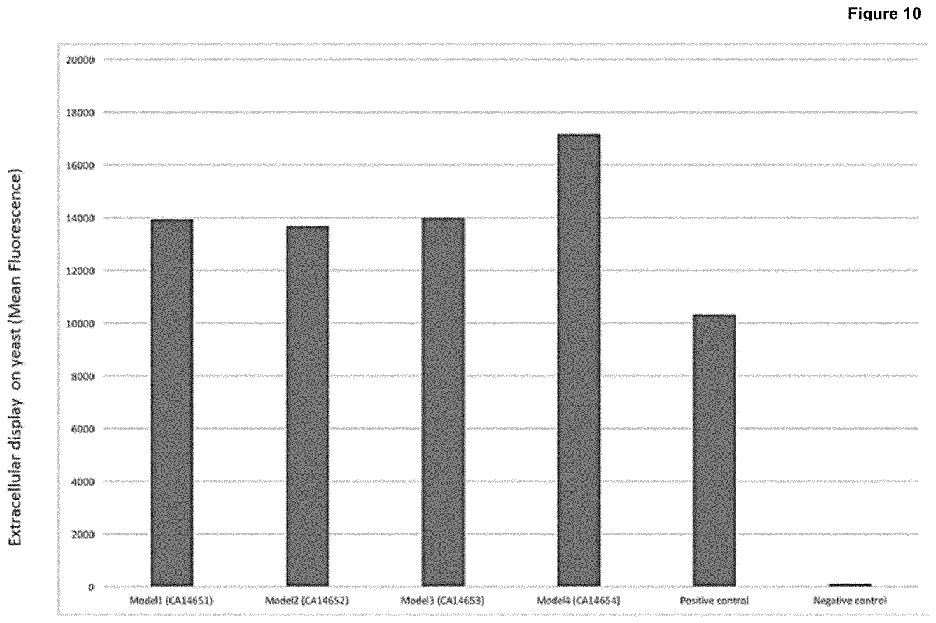

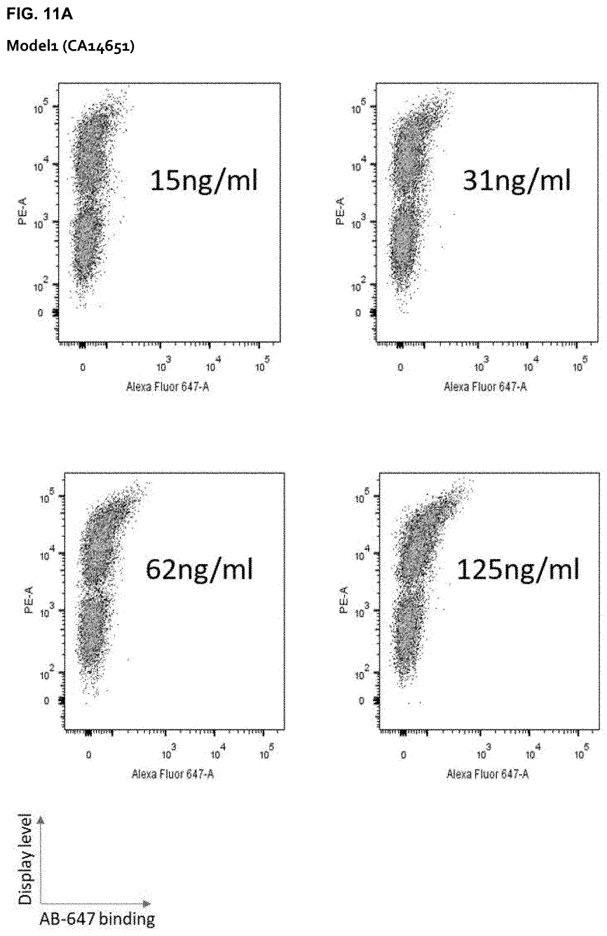

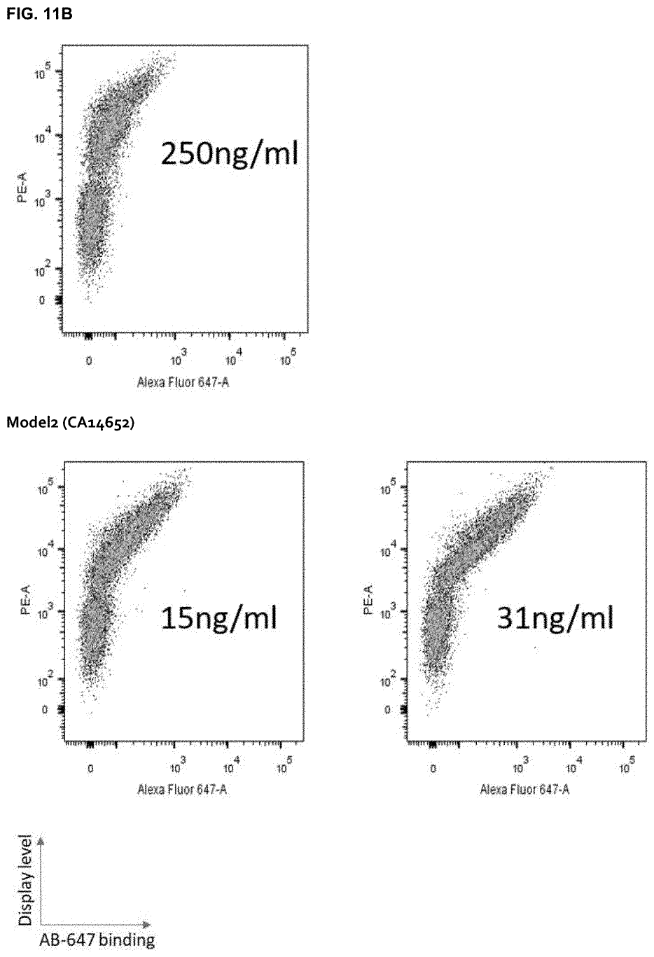

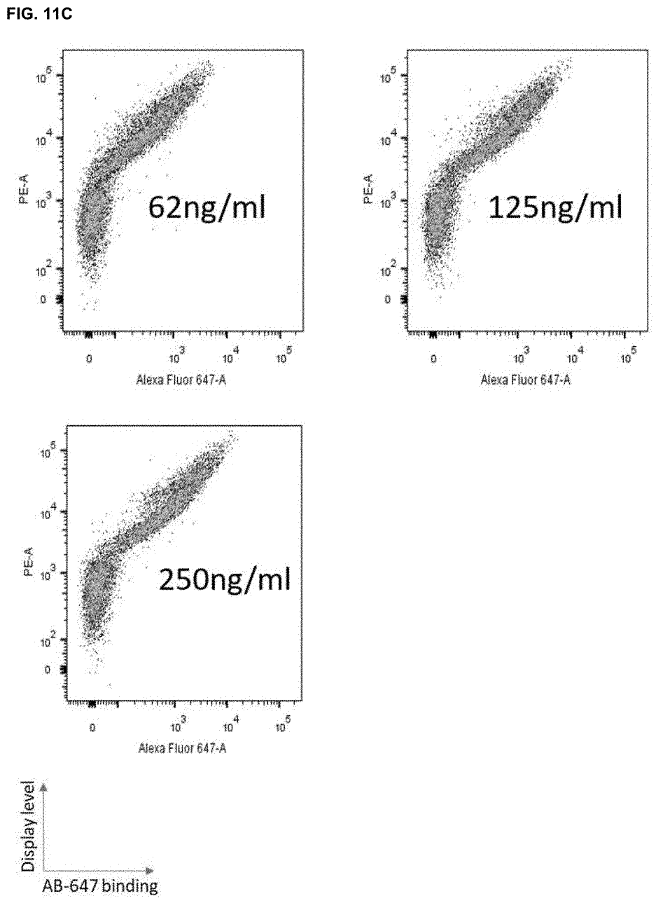

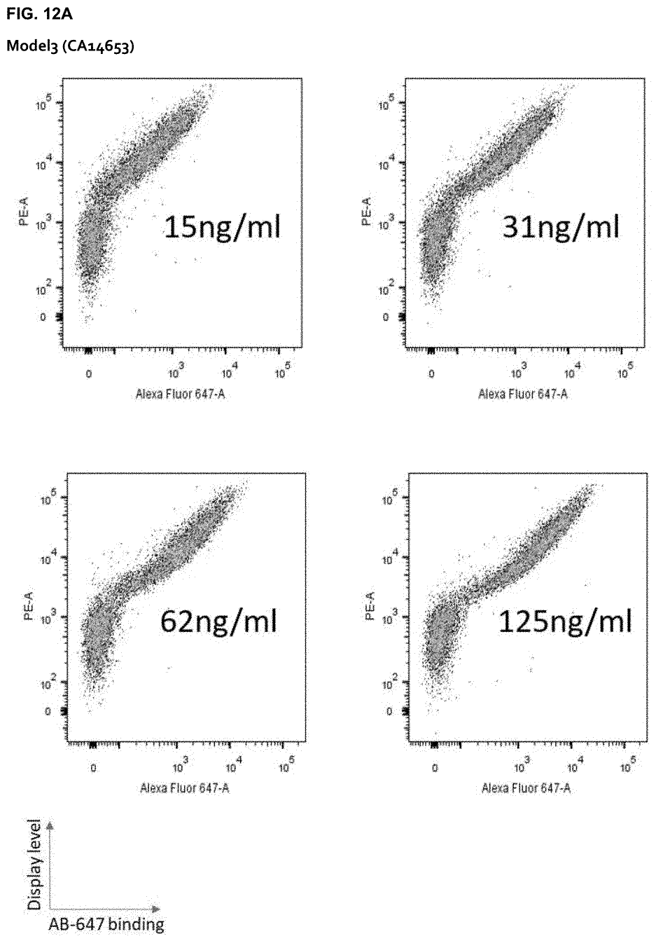

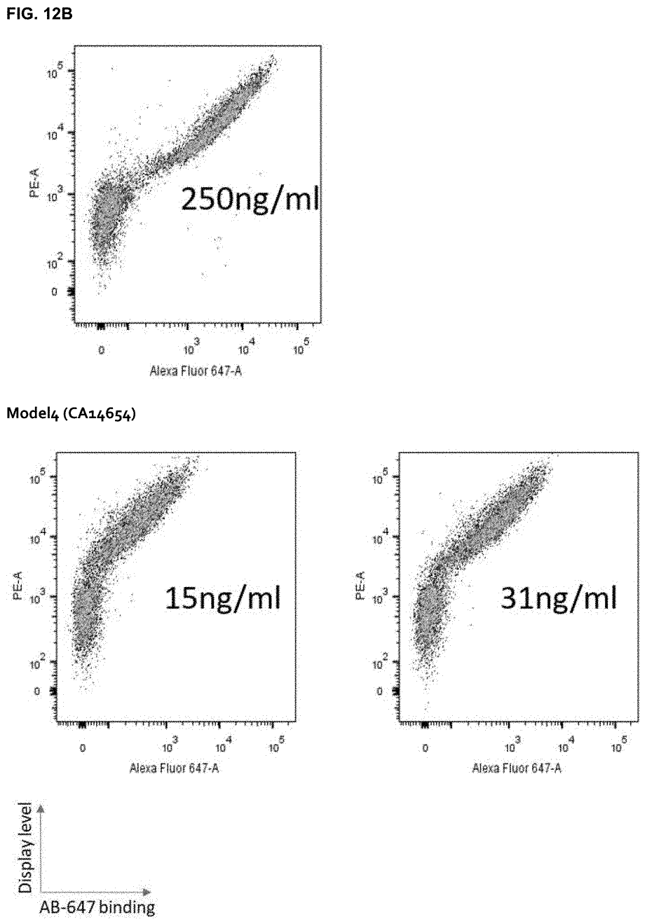

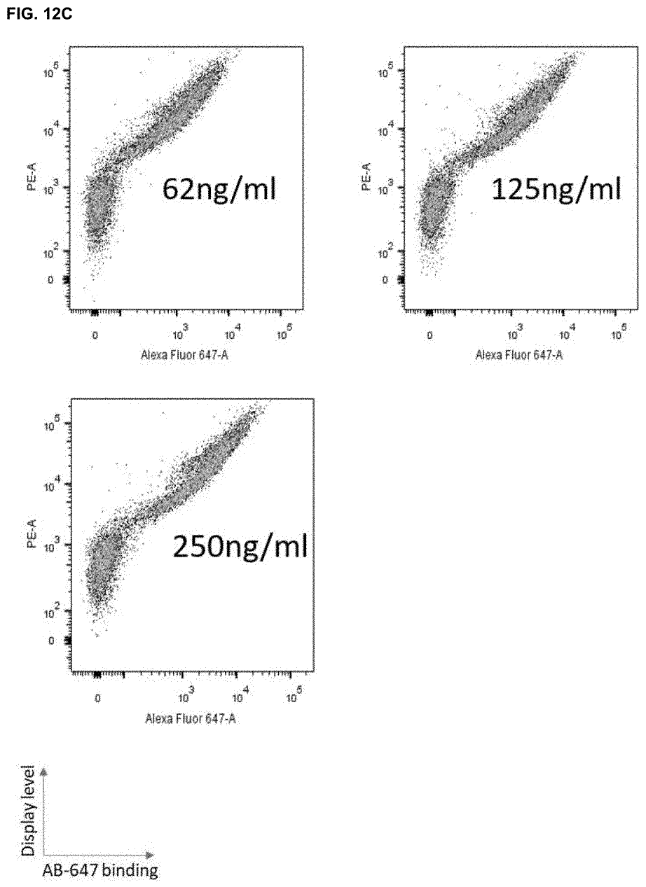

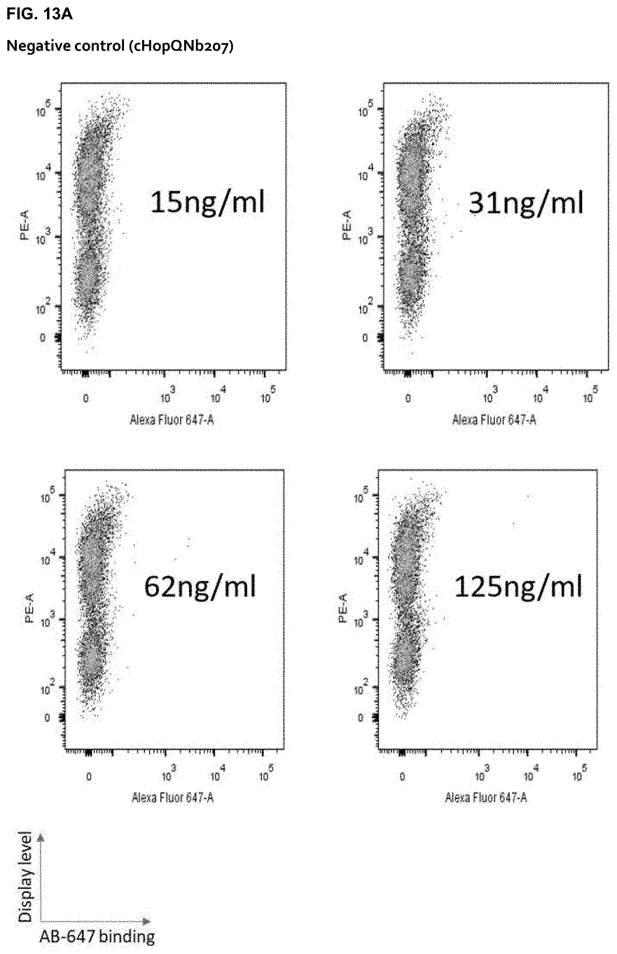

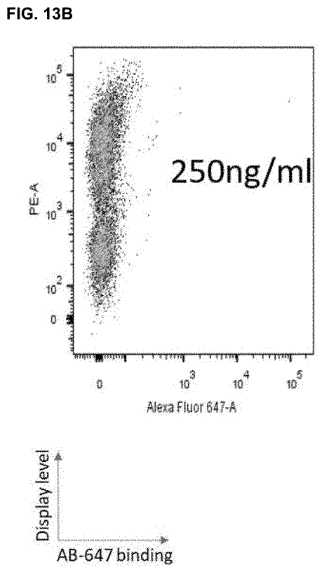

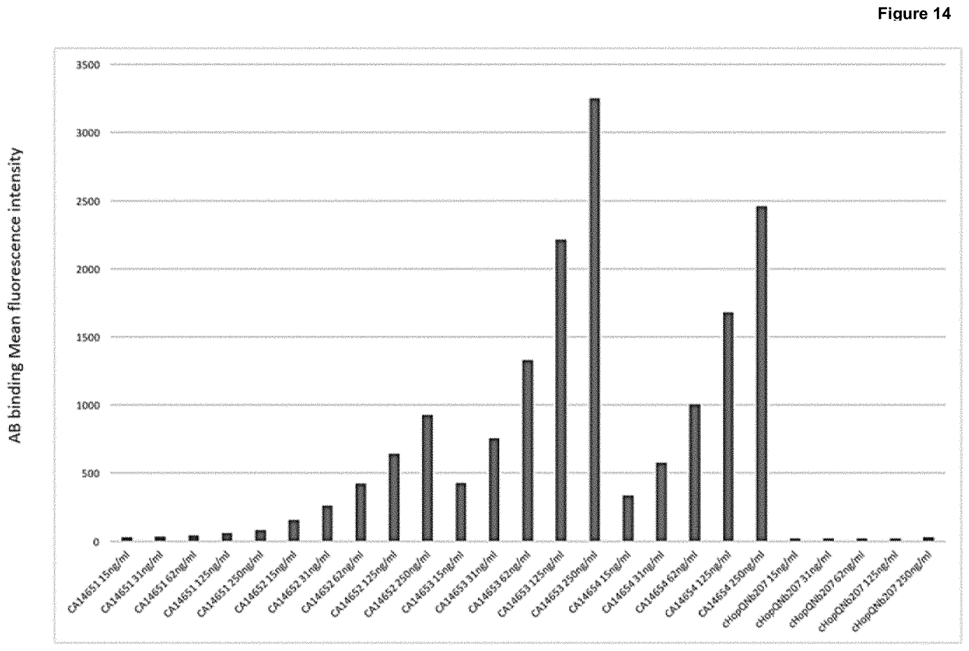

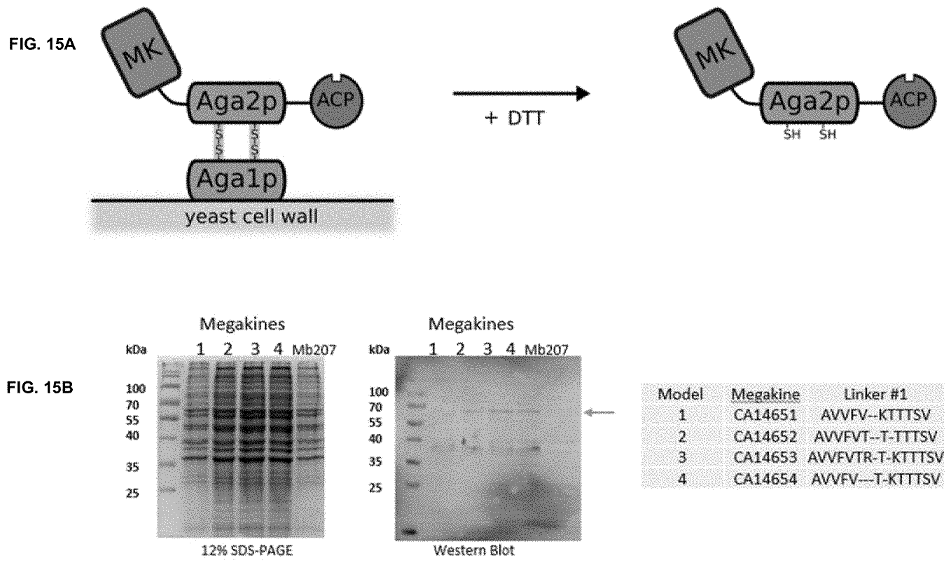

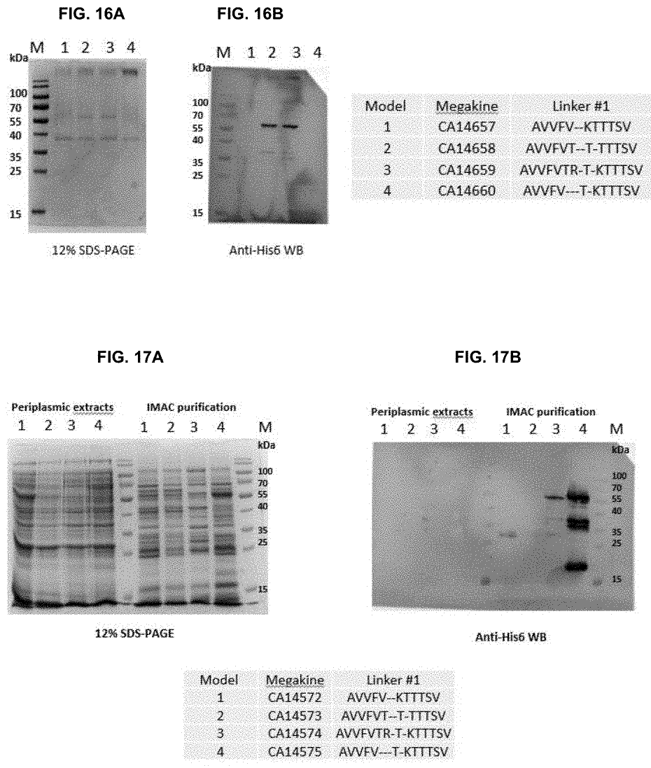

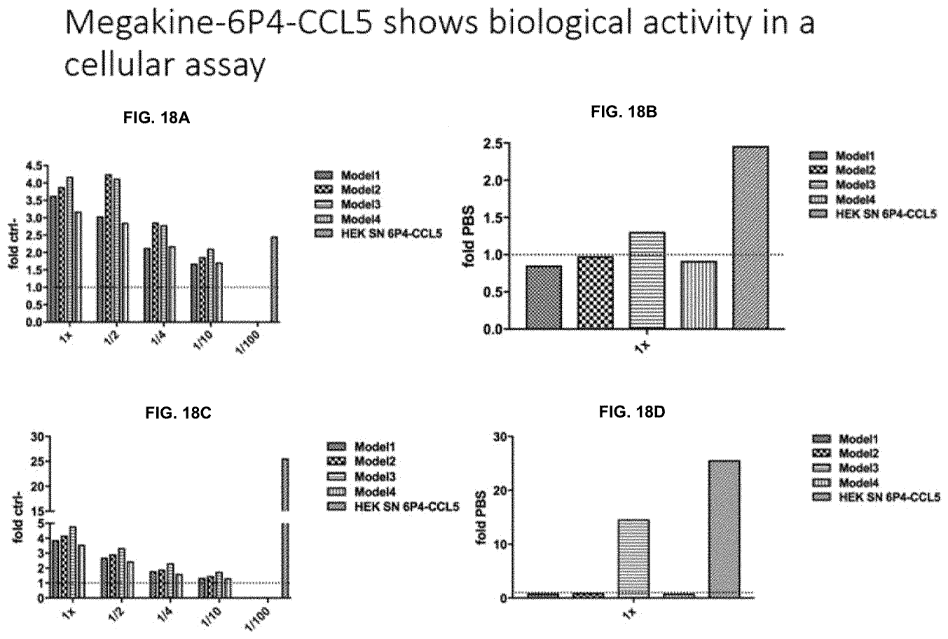

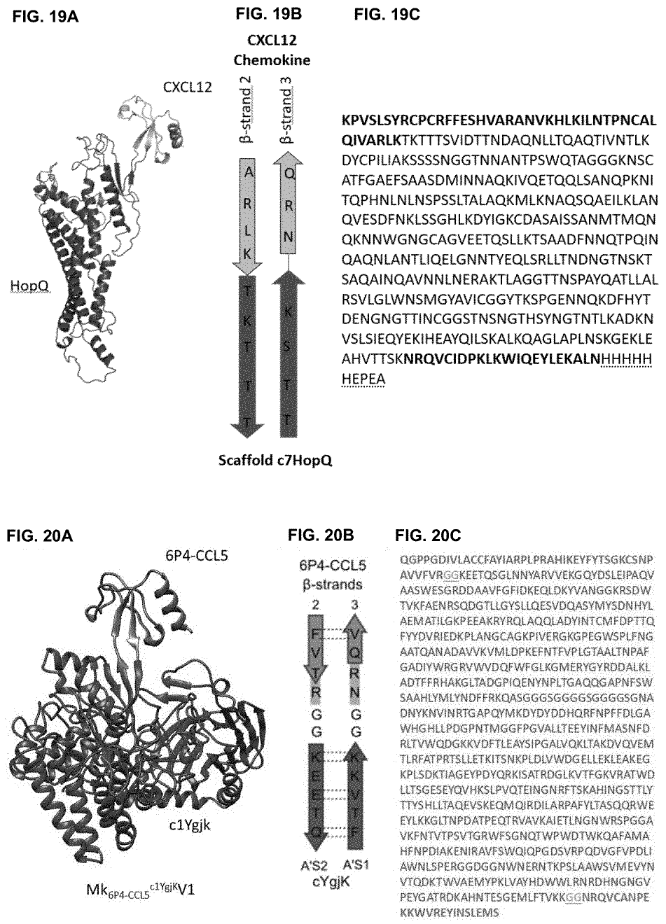

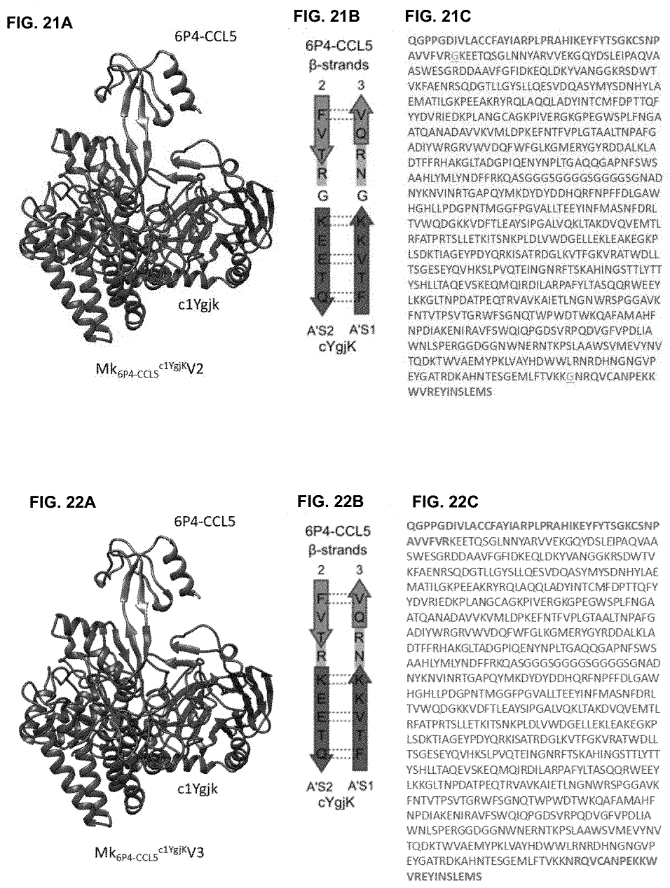

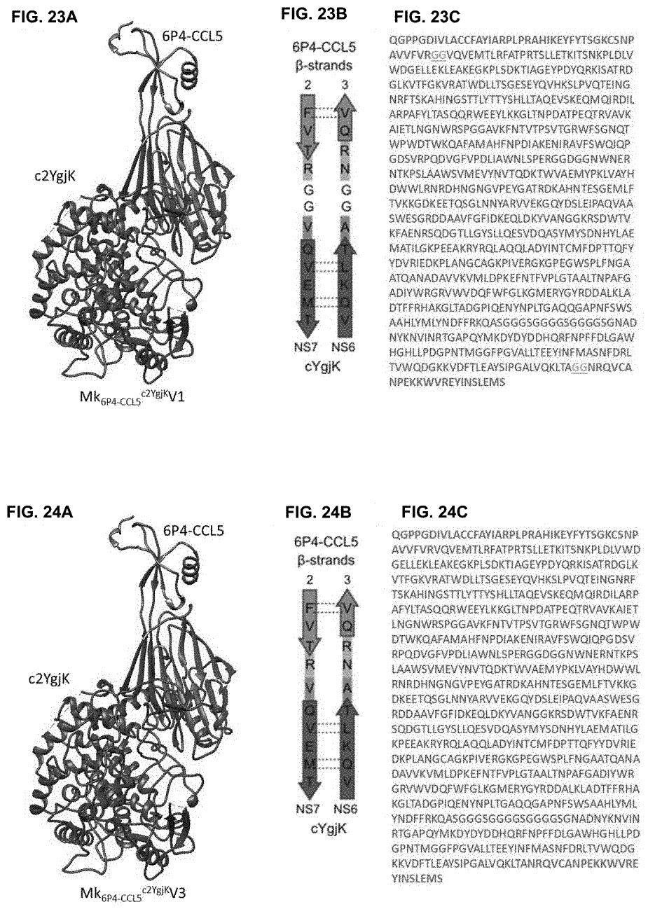

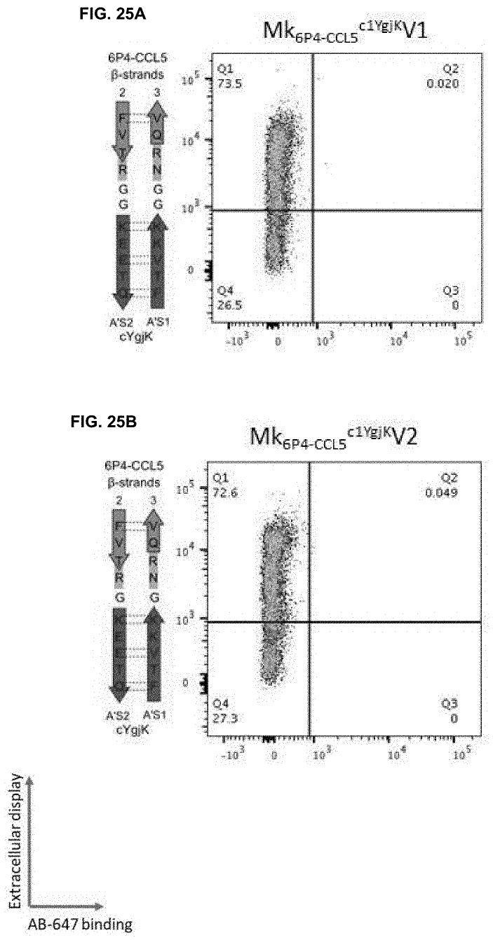

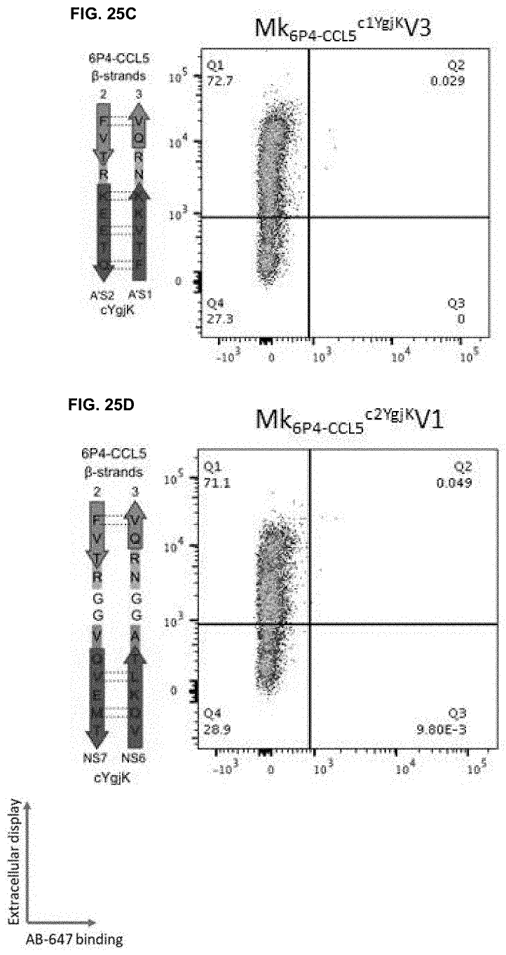

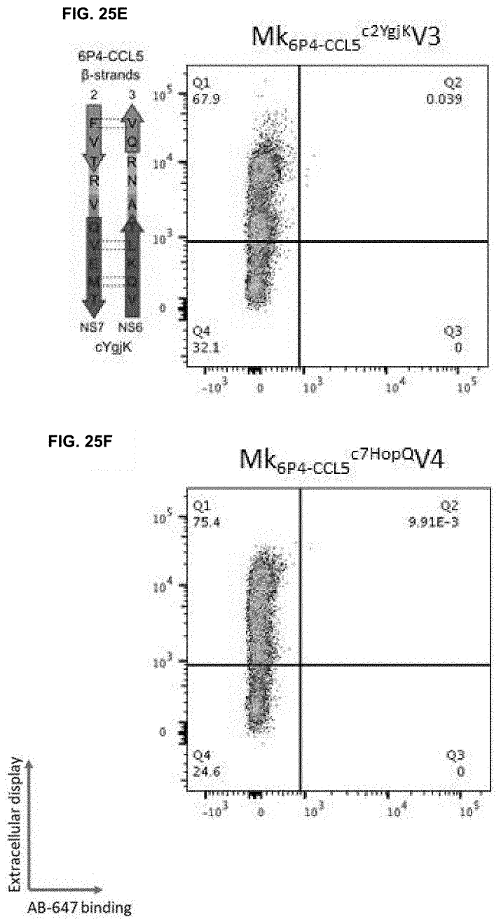

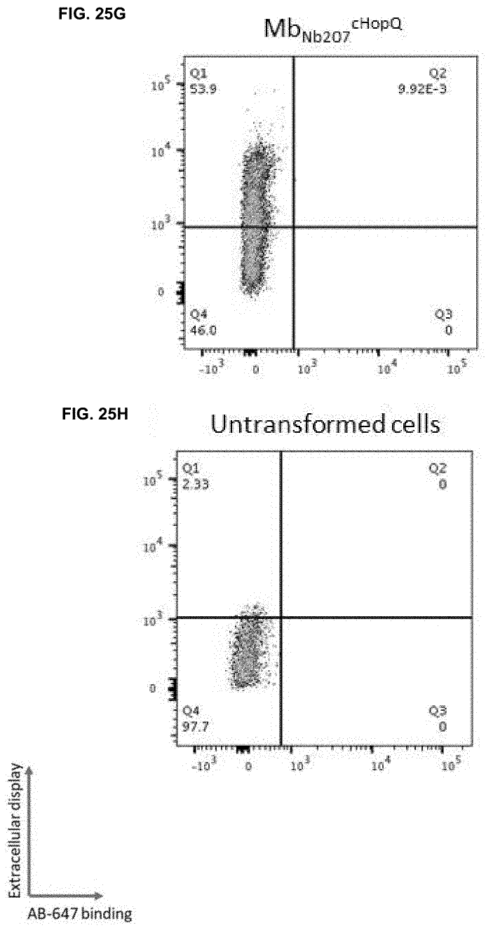

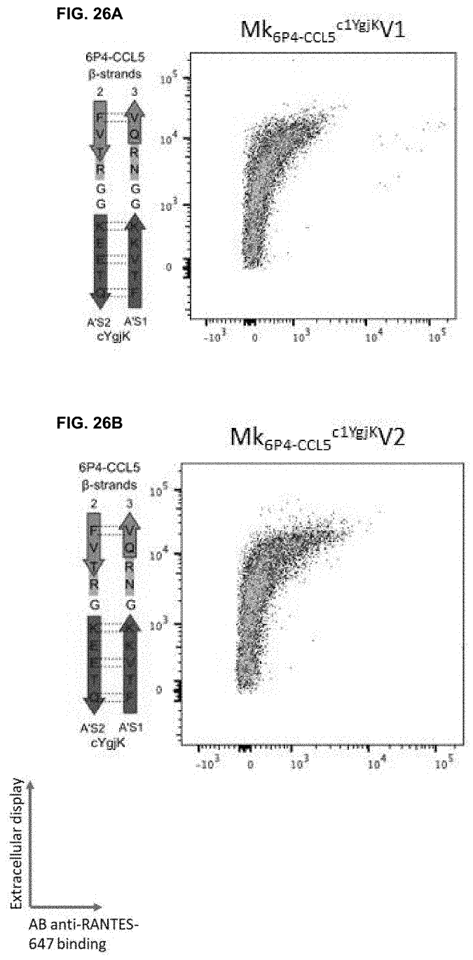

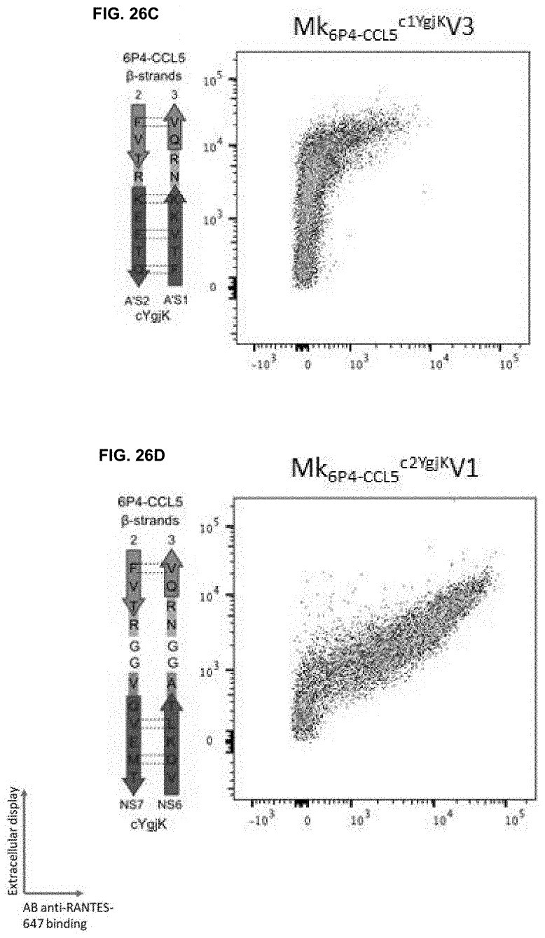

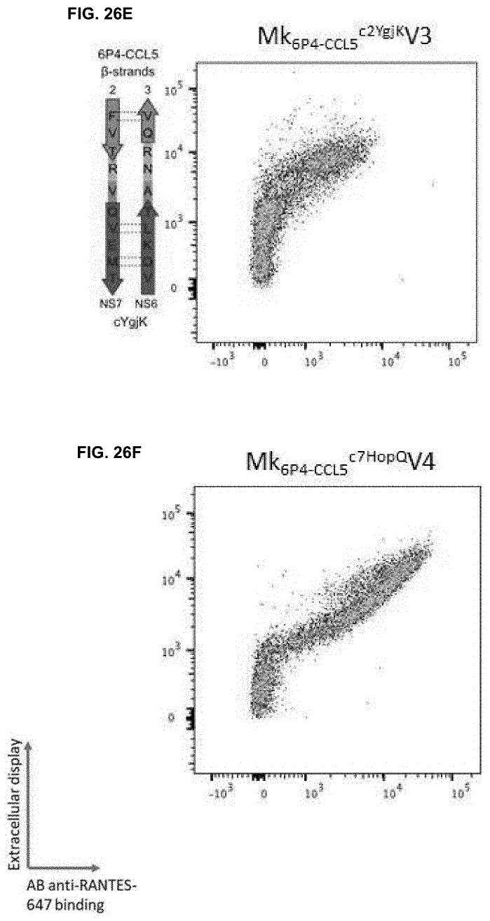

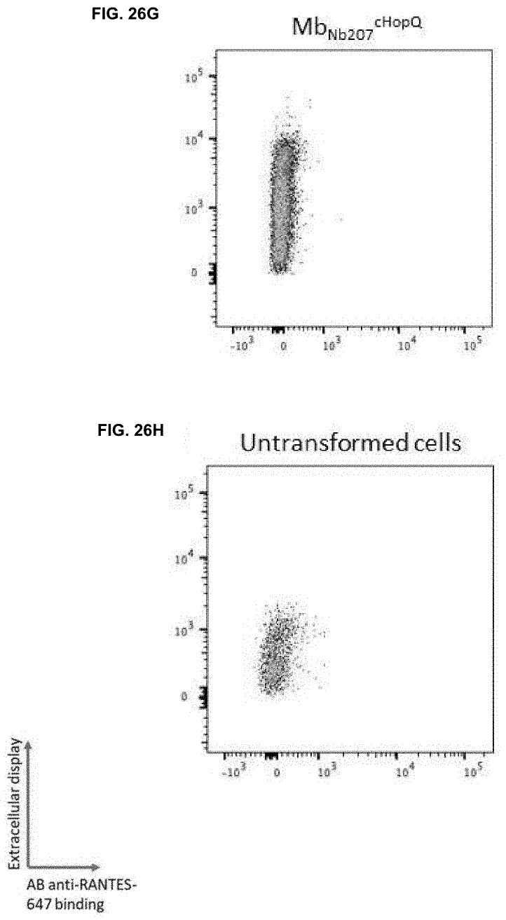

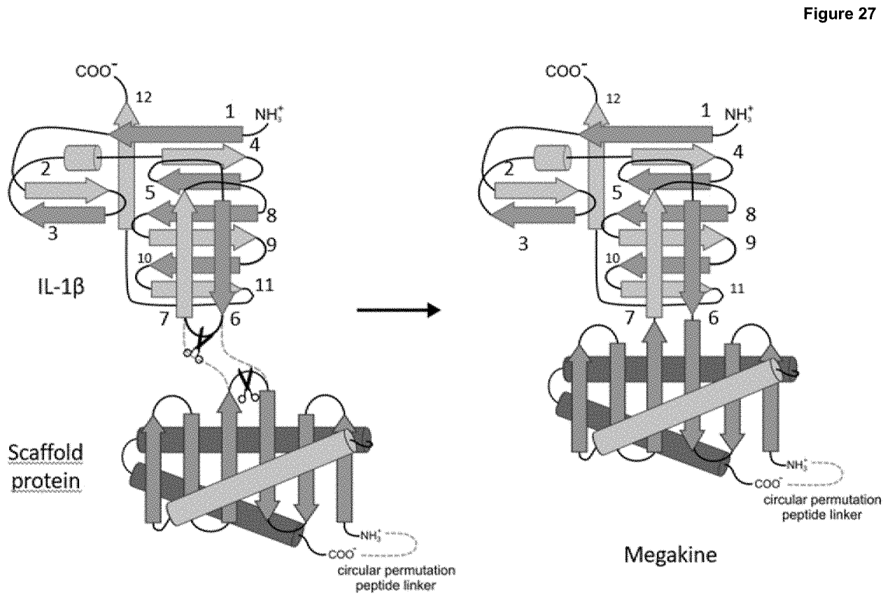

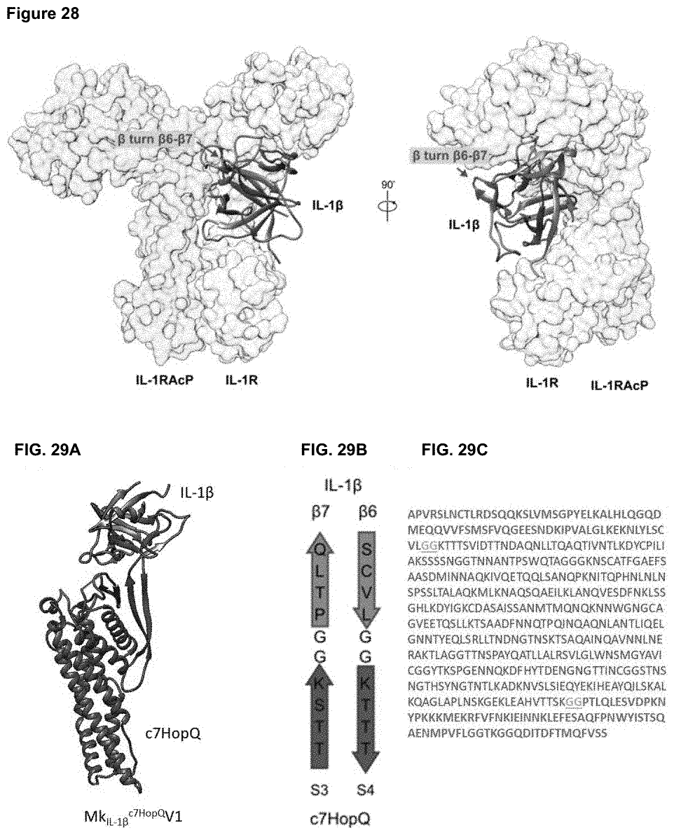

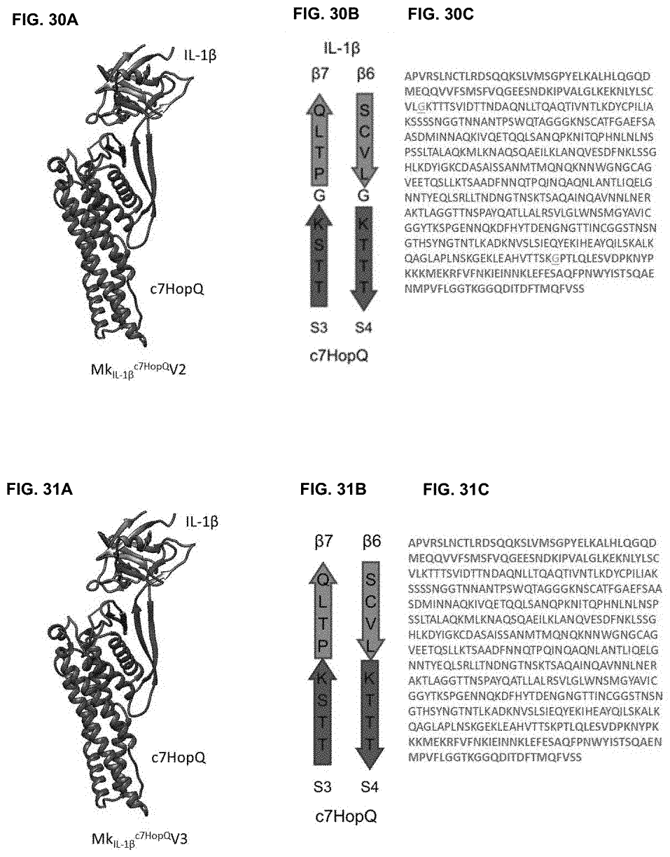

(FIG. 3A) Model of a chemokine fusion protein made by fusion of a chemokine 6P4-CCL5 (top) and a circularly permutated variant of the Adhesin domain of HopQof H. pylori (bottom) via two peptide bonds or linkers that connect the chemokine to the scaffold. (FIG. 3B) A circularly permutated gene encoding the Adhesin domain of the type 1 HopQ of Helicobacter pylori strain G27 (bottom, PDB 5LP2, SEQ ID NO: 2, c7HopQ) was inserted in the β-turn of 6P4-CCL5 (top, PDB 5UIW, SEQ ID NO: 1) connecting β-strands β2 to β3 (β-turn β2-β3). (FIG. 3C) Amino acid sequence of the resulting chemokine chimeric protein (Mk_{6P4-CCL5}^{c7HopQ}, SEQ ID NO: 3). Sequences originating from the chemokine are depicted in bold. Sequences originating from HopQ are in between. The C-terminal tag includes 6× His and EPEA are dashed underlined.

FIGS. 4A-4C. Model 2 of a 50 kD 6P4-CCL5 fusion protein built from a circularly permutated variant of HopQ inserted into the β-turn connecting β-strands β2 and β3 of the 6P4-CCL5 chemokine.

(FIG. 4A) Model of a chemokine fusion protein made by fusion of a chemokine 6P4-CCL5 (top,) and a circularly permutated variant of the Adhesin domain of HopQ of H. pylori (bottom,) via two peptide bonds or linkers that connect the chemokine to the scaffold. (FIG. 4B) A circularly permutated gene encoding the Adhesin domain of the type 1 HopQ of Helicobacter pylori strain G27 (bottom, PDB 5LP2, SEQ ID NO: 2, c7HopQ) was inserted in the β-turn of 6P4-CCL5 (top, PDB 5UIW, SEQ ID NO: 1) connecting β-strands β2 to β3 (β-turn β2-β3). (FIG. 4C) Amino acid sequence of the resulting chemokine chimeric protein (Mk_{6P4-CCL5}^{c7HopQ}, SEQ ID NO: 4). Sequences originating from the chemokine are depicted in bold. Sequences originating from HopQ are in between. The C-terminal tag includes 6× His and EPEA are dashed underlined.

FIGS. 5A-5C. Model 3 of a 50 kD 6P4-CCL5 fusion protein built from a circularly permutated variant of HopQ inserted into the β-turn connecting β-strands β2 and β3 of the 6P4-CCL5 chemokine.

(FIG. 5A) Model of a chemokine fusion protein made by fusion of a chemokine 6P4-CCL5 (top) and a circularly permutated variant of the Adhesin domain of HopQ of H. pylori (bottom) via two peptide bonds or linkers that connect the chemokine to the scaffold. (FIG. 5B) A circularly permutated gene encoding the Adhesin domain of the type 1 HopQ of Helicobacter pylori strain G27 (bottom, PDB 5LP2, SEQ ID NO: 2, c7HopQ) was inserted in the β-turn of 6P4-CCL5 (top, PDB 5UIW, SEQ ID NO: 1) connecting β-strands β2 to β3 (β-turn β2-β3). (FIG. 5C) Amino acid sequence of the resulting chemokine fusion protein (Mk_{6P4-CCL5}^{7HopQ}, SEQ ID NO: 5). Sequences originating from the chemokine are depicted in bold. Sequences originating from HopQ are in between. The C-terminal tag includes 6× His and EPEA are dashed underlined.

FIGS. 6A-6C. Model 4 of a 50 kD 6P4-CCL5 fusion protein built from a circularly permutated variant of HopQ inserted into the β-turn connecting β-strands β2 and β3 of the 6P4-CCL5 chemokine.

(FIG. 6A) Model of a chemokine fusion protein made by fusion of a chemokine 6P4-CCL5 (top) and a circularly permutated variant of the Adhesin domain of HopQ of H. pylori (bottom) via two peptide bonds or linkers that connect the chemokine to the scaffold. (FIG. 6B) A circularly permutated gene encoding the Adhesin domain of the type 1 HopQ of Helicobacter pylori strain G27 (bottom, PDB 5LP2, SEQ ID NO: 2, c7HopQ) was inserted in the β-turn of 6P4-CCL5 (top, PDB 5UIW, SEQ ID NO: 1) connecting β-strands β2 to β3 (β-turn β2-β3). (FIG. 6C) Amino acid sequence of the resulting chemokine fusion protein (Mk_{6P4-CCL5}^{7HopQ}, SEQ ID NO: 6). Sequences originating from the chemokine are depicted in bold. Sequences originating from HopQ are in between. The C-terminal tag includes 6× His and EPEA are dashed underlined.

FIGS. 7A-7C. Yeast display vector for the optimization of the composition and the length of the linker peptides connecting scaffold protein HopQ to a chemokine.

(FIG. 7A) Schematic representation of the display vector. LS: the engineered secretion signal of yeast α-factor, appS4 (Rakestraw et al. 2009) that directs extracellular secretion in yeast. N: N-terminal part of the 6P4-CCL5 chemokine until β-strand β2 (1-43 of SEQ ID NO: 1); circularly permutated gene encoding the Adhesin domain of the type 1 HopQ of Helicobacter pylori strain G27 (bottom, PDB 5LP2, SEQ ID NO: 2, c7HopQ); 6P4-CCL5 C-terminus from β-strand β3 of the 6P4-CCL5 chemokine (47-69 of SEQ ID NO: 1); a flexible linker connecting to the displayed protein Aga2p, the adhesion subunit of the yeast agglutinin protein which attaches to the yeast cell wall through disulfide bonds to Aga1p protein (Chao et al., 2006); ACP: Acyl carrier protein for the orthogonal labelling of the displayed chemokine fusion protein to monitor its expression level (Johnsson et al., 2005). (FIG. 7B) Sequence diversity of the displayed chemokine fusion proteins (SEQ ID NO: 25-28): AppS4 leader sequence in normal print, Megakine Mk_{6P4-CCL5}^{c7HopQ} with random linkers depicted in bold, (X)₁₋₂ is a short peptide linker of variable length (1 or 2 amino acids) and mixed composition, flexible (GGGS)ₙ polypeptide linker in italics, Aga2p protein sequence underlined, ACP sequence double underlined, cMyc Tag. (FIG. 7C) By using equimolar mixtures of 2 forward (SEQ ID NO: 29, SEQ ID NO: 30) and 2 reverse PCR primers (SEQ ID NO: 31, SEQ ID NO: 32) to introduce the short peptide linkers of variable length (1 or 2 amino acids) and mixed composition, 4 pools of chemokine fusion protein sequences were generated (each representing 25% of the library), encoding a total of 176400 AA-sequence variants.

FIG. 8. Consecutive rounds of selection of chemokine fusion proteins by yeast display and two-dimensional flow cytometry.

To optimize the composition and the length of the linker peptides connecting scaffold protein HopQ to chemokine CCL5, selection was performed by Yeast display and flow cytometry. Each dot represents two fluorescent signals of a separate EBY100 yeast cell transformed with a pCTCON2 derivative encoding the chemokine fusion protein Mk_{6P4-CCL5}^{6HopQ} fused to Aga2p and ACP via linkers with a different length and composition. Yeast cells were orthogonally stained with CoA-547 (2 μM) using the SFP synthase (1 μM) to measure the Megakine display level (Y-axis). To measure if the displayed Megakine contains a folded CCL5 moiety, these cells were supplementary stained with an Alexa Fluor® 647 labelled anti-human RANTES (CCL5) Antibody (X-axis). In round 1, the library was incubated with 0.25 mg/ml of Alexa Fluor® 647 anti-human RANTES (CCL5) Antibody. 200000 yeast cells displaying a high fluorescence for Mega kine expression (PE channel) and anti-human RANTES (CCL5) (647 nm channel) were sorted. In round 2, we incubated the enriched library with 0.025 mg/ml of Alexa Fluor® 647 anti-human RANTES (CCL5) Antibody. 20000 yeast cells displaying the highest fluorescence for Megakine expression (PE channel) and anti-human RANTES (CCL5) (647 nm channel) were sorted and subjected to sequence analysis.

FIGS. 9A-9F. Qualitative analysis of the display of four different chemokine fusion proteins with different linkers on the surface of EBY100 yeast cells by two-dimensional flow cytometry.

Dot plot representation of the relative fluorescence intensity of individual EBY100 yeast cells transformed with a pCTCON2 derivative encoding the chemokine fusion protein Mk_{6P4-CCL5}^{c7HopQ} fused to Aga2p and ACP (FIGS. 9A to 9D, Models 1 to 4, respectively, SEQ ID NO: 7-10). Yeast cells displaying MegaBody Mb_{Nb207}^{cHopQ} were used as the positive control (FIG. 9E, SEQ ID NO: 11). Untransformed EBY100 yeast cells were included as the negative control in this experiment (FIG. 9F). Transformed and untransformed yeast cells were orthogonally stained equally with CoA-547 (2 μM) using the SFP synthase (1 μM).

FIG. 10. Quantitative analysis of the display of four different chemokine fusion proteins with different linkers on the surface of EBY100 yeast cells by flow cytometry.

The single-parameter histograms show the relative fluorescence intensity of EBY100 yeast cells transformed with a pCTCON2 derivative encoding the chemokine fusion protein Mk_{6P4-CCL5}^{c7HopQ} fused to Aga2p and ACP (Version 1 to 4, SEQ ID NO: 7-10) compared Mb_{Nb207}^{cHopQ} as positive control (SEQ ID NO: 11) and to untransformed EBY100 yeast cells as negative control. Transformed and untransformed yeast cells were orthogonally stained with CoA-547 (2 μM) using the SFP synthase (1 μM). Model 1,2,3,4 refers to the actual clones or fusion proteins.

FIGS. 11A-11C. Flow cytometric analysis of the functionality of Mk_{6P4-CCL5}^{c7HopQ} fusion protein variants 1 and 2 displayed on the surface of EBY100 yeast cells.

Dot plot representation of the relative fluorescence intensity of individual EBY100 yeast cells transformed with a pCTCON2 derivative encoding the Mk_{6P4-CCL5}^{c7HopQ} fusion protein Models 1 and 2 as Aga2p and ACP fusions (SEQ ID NO: 7 and SEQ ID NO: 8). Yeast clones were induced and orthogonally stained with CoA-547 (2 μM) using the SFP synthase (1 μM) and incubated with five different concentrations of Alexa Fluor® 647 anti-human RANTES (CCL5) Antibody (15, 31, 62, 125 and 250 ng/mL, respectively) (FIGS. 11A-11C). The y-axis displays the mean fluorescence intensity of relative PE/CoA-547 fluorescence (Megakine display level). The x-axis displays the mean fluorescence intensity of relative Alexa Fluor° 647 anti-human fluorescence RANTES (CCL5) Antibody binding. Models 1,2 refer to the actual clones.

FIGS. 12A-12C. Flow cytometric analysis of the functionality of Mk_{6P4-CCL5}^{c7HopQ} fusion protein variants 3 and 4 displayed on the surface of EBY100 yeast cells.

Dot plot representation of the relative fluorescence intensity of individual EBY100 yeast cells transformed with a pCTCON2 derivative encoding the Mk_{6P4-CCL5}^{c7HopQ} fusion protein Models 3 and 4 as Aga2p and ACP fusions (SEQ ID NO: 9 and SEQ ID NO: 10). Yeast clones were induced and orthogonally stained with CoA-547 (2 μM) using the SFP synthase (1 μM) and incubated with five different concentrations of Alexa Fluor® 647 anti-human RANTES (CCL5) Antibody (15, 31, 62, 125 and 250 ng/mL, respectively) (FIGS. 12A-12C). The y axis displays the mean fluorescence intensity of relative PE/CoA-547 fluorescence (Megakine display level), the x axis displays the mean fluorescence intensity of relative Alexa Fluor® 647 fluorescence (RANTES (CCL5) Antibody binding). Models 3,4 refer to the actual clones.

FIGS. 13A-13B. Flow cytometric analysis of the functionality of antigen-binding chimeric protein Mb_{Nb207}^{cHopQ} displayed on the surface of EBY100 yeast cells.

Dot plot representation of the relative fluorescence intensity of individual EBY100 yeast cells transformed with a pCTCON2 derivative encoding the antigen-binding chimeric protein Mb_{Nb207}^{cHopQ} as Aga2p and ACP fusion (SEQ ID NO: 11). Yeast clones were induced and orthogonally stained with CoA-547 (2 μM) using the SFP synthase (1 μM) and incubated with five different concentrations of Alexa Fluor® 647 anti-human RANTES (CCL5) Antibody (15, 31, 62, 125 and 250 nem L, respectively) (FIGS. 13A-13B). The y axis displays the mean fluorescence intensity of relative PE/CoA-547 fluorescence (antigen-binding chimeric protein display level), the x-axis displays the mean fluorescence intensity of relative Alexa Fluor® 647 fluorescence (RANTES (CCL5) Antibody binding).

FIG. 14. Flow cytometric quantitative analysis of the binding of four different chimeric chemokines to Alexa Fluor® 647 fluorescence RANTES (CCL5).

Chart representation of the calculated mean fluorescence intensities of relative Alexa Fluor® 647 fluorescence (RANTES (CCL5) Antibody binding) of individual EBY100 yeast cells transformed with a pCTCON2 derivative encoding the Mk_{6P4-CCL5}^{c7HopQ} fusion protein Models 1 to 4 (SEQ ID NO: 7-10) and negative control antigen-binding chimeric protein Mb_{Nb207}^{cHopQ} (SEQ ID NO: 11) as Aga2p and ACP fusions. Yeast clones were induced and incubated with five different concentrations of Alexa Fluor® 647 anti-human RANTES (CCL5) Antibody (15, 31, 62, 125 and 250 ng/mL, respectively).

FIGS. 15A and 15B. Displayed chemokine fusion proteins can be eluted from the yeast membrane.

(FIG. 15A) Schematic representation of the chemokine fusion proteins displayed on the yeast membrane and eluted using 1 mM DTT. (FIG. 15B) 12% SDS-PAGE, eluted fraction of the four different variant and antigen-binding chimeric protein Mb_{Nb207}^{cHopQ} as a control. Western blot analysis of the same gel using primary mouse anti-cMYC and goat anti-mouse Alkaline Phosphatase conjugate antibodies. The molecular mass of about 50 kDa for Mk_{6P4-CCL5}^{c7HopQ} was confirmed by molecular mass marker (arrow).

FIGS. 16A and 16B. SDS-PAGE and Western blot analysis of the expression of four different recombinant chemokine fusion protein variants secreted from S. cerevisiae EBY100.

His-tagged fusion protein Mk_{6P4-CCL5}^{cHopQ} Models 1 to 4 (SEQ ID NO: 12-15) were expressed in S. cerevisiae EBY100 fused to the appS4 leader sequence that directs extracellular secretion in yeast and purified by nickel affinity chromatography (IMAC). (FIG. 16A) IMAC purified fusion proteins Mk_{6P4-CCL5}^{c7HopQ} eluted with 500 mM imidazole, loaded on a 12% SDS-PAGE gel. (FIG. 16B) Western blot analysis of the same gel using primary mouse anti-His and goat anti-mouse Alkaline Phosphatase conjugate antibodies. The molecular mass of about 50 kDa for Mk_{6P4-CCL5}^{c7HopQ} was confirmed by molecular mass marker (left line: M).

FIGS. 17A and 17B. SDS-PAGE and Western blot analysis of the expression of four different recombinant chemokine fusion protein variants in the periplasm of E. coli WK6.

His-tagged fusion protein Mk_{6P4-CCL5}^{c7HopQ} Models 1 to 4 (SEQ ID NO: 3-6) were expressed in the periplasm of E. coli and purified by nickel affinity chromatography (IMAC). (FIG. 17A) Samples of fusion proteins Mk_{6P4-CCL5}^{c7HopQ} from E. coli periplasmic extracts and from purified proteins eluted with 500 mM imidazole after IMAC, loaded on a 12% SDS-PAGE gel. (FIG. 17B) Western blot analysis of the same gel using primary mouse anti-His and goat anti-mouse Alkaline Phosphatase conjugate antibodies. The molecular mass of about 50 kDa for Mk_{6P4-CCL5}^{c7HopQ} was confirmed by molecular mass marker (right line: M).

FIGS. 18A-18D. Biological activity of Mk_{6P4-CCL5}^{c7HopQ} V1-V4 fusion protein variants towards the chemokine receptor CCR5.

The recruitment of miniGi to CCR5 induced by chemokine fusion protein variants produced in the periplasm of E. coli at different dilutions (FIG. 18A) or following Ni-NTA purification (FIG. 18B) was monitored in HEK293T cells using a NanoLuc-complementation-assay. Recombinant soluble 6P4-CCL5 chemokine produced in HEK293T and diluted 100-fold was used as positive control. Results are represented as fold increase in luminescence over untreated samples.

The recruitment of β-arrestin-1 to CCR5 induced by chemokine fusion protein variants produced in the periplasm of E. coli at different dilutions (FIG. 18C) or following Ni-NTA purification (FIG. 18D) was monitored in HEK293T cells using a NanoLuc-complementation-assay. Recombinant soluble 6P4-CCL5 chemokine produced in HEK293T and diluted 100-fold was used as positive control. Results are represented as fold increase in luminescence over untreated samples.

FIGS. 19A-19C. Model of a 50 kD CXCL12 fusion protein built from a circularly permutated variant of HopQ inserted into the β-turn connecting β-strands β2 and β3 of the CXCL12 chemokine.

(FIG. 19A) Model of a chemokine fusion protein made by fusion of CXCL12 (top) and a circularly permutated variant of the Adhesin domain of HopQ of H. pylori (bottom) via two peptide bonds or linkers that connect chemokine to scaffold. (FIG. 19B) A circularly permutated gene encoding the Adhesin domain of the type 1 HopQ of Helicobacter pylori strain G27 (bottom, PDB 5LP2, SEQ ID NO: 2, c7HopQ) was inserted in the β-turn of CXCL12 (top, SEQ ID NO: 22) connecting β-strands β2 to β3 (β-turn β2-β3). (FIG. 19C) Amino acid sequence of the resulting CXCL12 chemokine fusion protein (Mk_{CXCL12}^{c7HopQ}, SEQ ID NO: 23). Sequences originating from the chemokine are depicted in bold. Sequences originating from HopQ are in normal text. The C-terminal tag includes 6× His and EPEA are underlined with a dotted line.

FIGS. 20A-20C. Model of Mk_{6P4-CCL5}^{c1YgjK}V1, a 94 kD 6P4-CCL5 fusion protein built from a circularly permutated c1YgjK variant inserted into the β-turn connecting β-strands β2 and β3 of the 6P4-CCL5 chemokine.

(FIG. 20A) Model of a chemokine fusion protein made by fusion of a chemokine 6P4-CCL5 (top) and a circularly permutated variant of the YgjK glycosidase of E. coli (bottom) via two peptide bonds or linkers that connect the chemokine to the scaffold. (FIG. 20B) A circularly permutated variant 1 gene encoding the YgjK glycosidase of E. coli (bottom, PDB 3W75, SEQ ID NO: 36, c1YgjK) was inserted in the β-turn of 6P4-CCL5 (top, PDB 5UIW, SEQ ID NO: 1) connecting β-strands β2 to β3 (β-turn β2-β3). (FIG. 20C) Amino acid sequence of the resulting chemokine chimeric protein (Mk_{6P4-CCL5}^{c1YgjK}V1, SEQ ID NO: 38). Sequences originating from the chemokine are depicted in bold. Two amino acid peptide linkers are underlined. Sequences originating from c1YgjK are in between.

FIGS. 21A-21C. Model of Mk_{6P4-CCL5}^{c1YgjK}V2, a 94 kD 6P4-CCL5 fusion protein built from a circularly permutated c1YgjK variant inserted into the β-turn connecting β-strands β2 and β3 of the 6P4-CCL5 chemokine.

(FIG. 21A) Model of a chemokine fusion protein made by fusion of a chemokine 6P4-CCL5 (top) and a circularly permutated variant of the YgjK glycosidase of E. coli (bottom) via two peptide bonds or linkers that connect the chemokine to the scaffold. (FIG. 21B) A circularly permutated variant 1 gene encoding the YgjK glycosidase of E. coli (bottom, PDB 3W7S, SEQ ID NO: 36, c1YgjK) was inserted in the β-turn of 6P4-CCL5 (top, PDB 5UIW, SEQ ID NO: 1) connecting β-strands β2 to β3 (β-turn β2-β3). (FIG. 21C) Amino acid sequence of the resulting chemokine chimeric protein (Mk_{6P4-CCL5}^{c1YgjK}V2, SEQ ID NO: 39). Sequences originating from the chemokine are depicted in bold. One amino acid peptide linkers are underlined. Sequences originating from c1YgjK are in between.

FIGS. 22A-22C. Model of Mk_{6P4-CCL5}^{c1YgjK}V3, a 94 kD 6P4-CCL5 fusion protein built from a circularly permutated c1YgjK variant inserted into the β-turn connecting β-strands β2 and β3 of the 6P4-CCL5 chemokine.

(FIG. 22A) Model of a chemokine fusion protein made by fusion of a chemokine 6P4-CCL5 (top) and a circularly permutated variant of the YgjK glycosidase of E. coli (bottom) via two peptide bonds or linkers that connect the chemokine to the scaffold. (FIG. 22B) A circularly permutated variant 1 gene encoding the YgjK glycosidase of E. coli (bottom, PDB 3W7S, SEQ ID NO: 36, c1YgjK) was inserted in the β-turn of 6P4-CCL5 (top, PDB 5UIW, SEQ ID NO: 1) connecting β-strands β2 to β3 (β-turn β2-β3). (FIG. 22C) Amino acid sequence of the resulting chemokine chimeric protein (Mk_{6P4-CCL5}^{c1YgjK}V3, SEQ ID NO: 40). Sequences originating from the chemokine are depicted in bold. Sequences originating from c1YgjK are in between.

FIGS. 23A-23C. Model of Mk_{6P4-CCL5}^{c2YgjK}V1, a 94 kD 6P4-CCL5 fusion protein built from a circularly permutated c2YgjK variant inserted into the β-turn connecting β-strands β2 and β3 of the 6P4-CCL5 chemokine.

(FIG. 23A) Model of a chemokine fusion protein made by fusion of a chemokine 6P4-CCL5 (top) and a circularly permutated variant of the YgjK glycosidase of E. coli (bottom) via two peptide bonds or linkers that connect the chemokine to the scaffold. (FIG. 23B) A circularly permutated variant B gene encoding the YgjK glycosidase of E. coli (bottom, PDB 3W7S, SEQ ID NO: 37, c2YgjK) was inserted in the β-turn of 6P4-CCL5 (top, PDB 5UIW, SEQ ID NO: 1) connecting β-strands β2 to β3 (β-turn β2-β3). (FIG. 23C) Amino acid sequence of the resulting chemokine chimeric protein (Mk_{6P4-CCL5}^{c2YgjK}V1, SEQ ID NO: 41). Sequences originating from the chemokine are depicted in bold. Two amino acid peptide linkers are underlined. Sequences originating from c2YgjK are in between.

FIGS. 24A-24C. Model of Mk_{6P4-CCL5}^{c2YgjK}V3, a 94 kD 6P4-CCL5 fusion protein built from a circularly permutated c2YgjK variant inserted into the β-turn connecting β-strands β2 and β3 of the 6P4-CCL5 chemokine.

(FIG. 24A) Model of a chemokine fusion protein made by fusion of a chemokine 6P4-CCL5 (top) and a circularly permutated variant of the YgjK glycosidase of E. coli (bottom) via two peptide bonds or linkers that connect the chemokine to the scaffold. (FIG. 24B) A circularly permutated variant 2 gene encoding the YgjK glycosidase of E. coli (bottom, PDB 3W7S, SEQ ID NO: 37, c2YgjK) was inserted in the β-turn of 6P4-CCL5 (top, PDB 5UIW, SEQ ID NO: 1) connecting β-strands β2 to β3 (β-turn β2-β3). (FIG. 24C) Amino acid sequence of the resulting chemokine chimeric protein (Mk_{6P4-CCL5}^{c2YgjK}V3, SEQ ID NO: 42). Sequences originating from the chemokine are depicted in bold. Sequences originating from c2YgjK are in between.

FIGS. 25A-25H. Qualitative analysis of the display of five different chemokine fusion proteins with different linkers and topologies on the surface of EBY100 yeast cells by two-dimensional flow cytometry.

Dot plot representation of the relative fluorescence intensity of individual EBY100 yeast cells transformed with a pCTCON2 derivative encoding the chemokine fusion protein Mk_{6P4-CCL5}^{c1YgjK}V1-V3 fused to Aga2p and ACP (FIGS. 25A to 25C, respectively, SEQ ID NO: 43-45) and Mk_{6P4-CCL5}^{c2YgjK}V1/V3 fused to Aga2p and ACP (FIGS. 25D to 25E, respectively, SEQ ID NO: 46-47). Yeast cells displaying megakine Mk_{6P4-CCL5}^{c7HopQ}V4 (SEQ ID NO: 10) were used as the positive control (FIG. 25F, SEQ ID NO: 11). Yeast cells displaying MegaBody Mb_{Nb207}^{cHopQ} (FIG. 25G, SEQ ID NO: 11) and untransformed EBY100 yeast cells (FIG. 25H) were included as the negative control in this experiment. Transformed and untransformed yeast cells were orthogonally stained equally with CoA-547 (2 μM) using the SFP synthase (1 μM).

FIGS. 26A-26H. Flow cytometric analysis of the functionality of Mk_{6P4-CCL5}^{c1/2YgjK} fusion protein variants displayed on the surface of EBY100 yeast cells.

Dot plot representation of the relative fluorescence intensity of individual EBY100 yeast cells transformed with a pCTCON2 derivative encoding the Mk_{6P4-CCL5}^{c1YgjK}V1-V3 fused to Aga2p and ACP (FIGS. 26A to 26C, respectively, SEQ ID NO: 43-45) and Mk_{6P4-CCL5}^{c2YgjK}V1/V3 fused to Aga2p and ACP (FIGS. 26D to 26E, respectively, SEQ ID NO: 46-47). Yeast cells displaying megakine Mk_{6P4-CCL5}^{c7HopQ}V4 (SEQ ID NO: 10) were used as the positive control (FIG. 26F, SEQ ID NO: 11). Yeast cells displaying MegaBody Mb_{Nb207}^{cHopQ} (FIG. 26G, SEQ ID NO: 11) and untransformed EBY100 yeast cells (FIG. 26H) were included as the negative control in this experiment. Yeast clones were induced and orthogonally stained with CoA-547 (2 μM) using the SFP synthase (1 μM) and incubated with Alexa Fluor® 647 anti-human RANTES (CCL5) Antibody at 80 ng/mL concentration. The y-axis displays the relative CoA-547 fluorescence (Megakine display level). The x-axis displays the relative Alexa Fluor® 647 anti-human fluorescence RANTES (CCL5) Antibody binding.

FIG. 27. Engineering principles of an interleukin fusion protein built from a circularly permutated variant of a scaffold protein that is inserted into the β-turn connecting β-strands β6 and β7 of a IL-1β interleukin.

This scheme shows how an interleukin can be grafted onto a large scaffold protein via two peptide bonds or two short linkers that connect the chemokine domain to the scaffold. Scissors indicate which exposed turns have to be cut in the interleukin and the scaffold. Dashed lines indicate how the remaining parts of the interleukin and the scaffold have to be concatenated by use of peptide bonds or short peptide linkers to build the interleukin chimeric protein.

FIG. 28. Crystal structure of IL-1β bound to the ectodomains of IL-1RII and IL-1RAcP.

IL-1β•IL-1RI•IL-1RAcP complex is presented in two views, with a rotation of 90° about the vertical axis. IL-1RII and IL-1RAcP are indicated as surface, IL-1β is indicated as ribbon structure. The β-turn connecting β-sheets β6 and β7 is highlighted by an arrow.

FIGS. 29A-29C. Model of Mk_{IL-1β}^{c7HopQ}V1, a 58 kD IL-1β fusion protein built from a circularly permutated HopQ variant inserted into the β-turn connecting β-strands β6 and β7 of the IL-1β interleukin.

(FIG. 29A) Model of a chemokine fusion protein made by fusion of the human IL-1β interleukin (top) and a circularly permutated variant of the adhesion domain of HopQ of H. pylori (bottom) via two peptide bonds or linkers that connect the interleukin to the scaffold. (FIG. 29B) A circularly permutated gene encoding the adhesion domain of HopQ of H. pylori (bottom, PDB 5LP2, SEQ ID NO: 2, c7HopQ) was inserted in the β-turn of IL-1β interleukin (top, PDB 3O4O, SEQ ID NO: 48) connecting β-strands β6 to β7 (β-turn β6-β7). (FIG. 29C) Amino acid sequence of the resulting interleukin chimeric protein (Mk_{IL-1β}^{cHopQ}V1, SEQ ID NO: 49). Sequences originating from the interleukin are depicted in bold. Two amino acid peptide linkers are underlined. Sequences originating from HopQ are in between.

FIGS. 30A-30C. Model of Mk_{IL-1β}^{c7HopQ}V2, a 58 kD IL-1β fusion protein built from a circularly permutated HopQ variant inserted into the β-turn connecting β-strands β6 and β7 of the IL-1β interleukin.

(FIG. 30A) Model of a chemokine fusion protein made by fusion of the human IL-1β interleukin (top) and a circularly permutated variant of the adhesion domain of HopQ of H. pylori (bottom) via two peptide bonds or linkers that connect the interleukin to the scaffold. (FIG. 30B) A circularly permutated gene encoding the adhesion domain of HopQ of H. pylori (bottom, PDB 5LP2, SEQ ID NO: 2, c7HopQ) was inserted in the β-turn of IL-1β interleukin (top, PDB 3O4O, SEQ ID NO: 48) connecting β-strands β6 to β7 (β-turn β6-β7). (FIG. 30C) Amino acid sequence of the resulting interleukin chimeric protein (Mk_{IL-1β}^{c7HopQ}V2, SEQ ID NO: 50). Sequences originating from the interleukin are depicted in bold. One amino acid peptide linkers are underlined. Sequences originating from HopQ are in between.

FIGS. 31A-31C. Model of Mk_{IL-1β}^{c7HopQ}V3, a 58 kD IL-1β fusion protein built from a circularly permutated HopQ variant inserted into the β-turn connecting β-strands β6 and β7 of the IL-1β interleukin.

(FIG. 31A) Model of a chemokine fusion protein made by fusion of the human IL-1β interleukin (top) and a circularly permutated variant of the adhesion domain of HopQ of H. pylori (bottom) via two peptide bonds or linkers that connect the interleukin to the scaffold. (FIG. 31B) A circularly permutated gene encoding the adhesion domain of HopQ of H. pylori (bottom, PDB 5LP2, SEQ ID NO: 2, c7HopQ) was inserted in the β-turn of IL-1β interleukin (top, PDB 3O4O, SEQ ID NO: 48) connecting β-strands β6 to β7 (β-turn β6-β7). (FIG. 31C) Amino acid sequence of the resulting interleukin chimeric protein (Mk_{IL-1β}^{c7HopQ}V3, SEQ ID NO: 51). Sequences originating from the interleukin are depicted in bold. Sequences originating from HopQ are in between.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, N.Y. (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods. ‘Similar’ as used herein, is interchangeable for alike, analogous, comparable, corresponding, and -like, and is meant to have the same or common characteristics, and/or in a quantifiable manner to show comparable results i.e. with a variation of maximum 20%, 10%, more preferably 5%, or even more preferably 1%, or less.

“Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. It also includes known types of modifications, for example, methylation, “caps” substitution of one or more of the naturally occurring nucleotides with an analog. By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like.

“Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances.

“Promoter region of a gene” as used here refers to a functional DNA sequence unit that, when operably linked to a coding sequence and possibly placed in the appropriate inducing conditions, is sufficient to promote transcription of said coding sequence. “Operably linked” refers to a juxtaposition wherein the components so described are in a relationship permitting them to function in their intended manner. A promoter sequence “operably linked” to a coding sequence is ligated in such a way that expression of the coding sequence is achieved under conditions compatible with the promoter sequence. “Gene” as used here includes both the promoter region of the gene as well as the coding sequence. It refers both to the genomic sequence (including possible introns) as well as to the cDNA derived from the spliced messenger, operably linked to a promoter sequence. The term “terminator” or “transcription termination signal” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

With a “genetic construct”, “chimeric gene”, “chimeric construct” or “chimeric gene construct” is meant a recombinant nucleic acid sequence in which a promoter or regulatory nucleic acid sequence is operatively linked to, or associated with, a nucleic acid sequence that codes for an mRNA, such that the regulatory nucleic acid sequence is able to regulate transcription or expression of the associated nucleic acid coding sequence. The regulatory nucleic acid sequence of the chimeric gene is not operatively linked to the associated nucleic acid sequence as found in nature. In particular, the term “genetic fusion construct” as used herein refers to the genetic construct encoding the mRNA that is translated to the fusion protein of the invention as disclosed herein.

The term “vector”, “vector construct,” “expression vector,” or “gene transfer vector,” as used herein, is intended to refer to a nucleic acid molecule capable of transporting another nucleic acid molecule to which it has been linked, and includes any vector known to the skilled person, including any suitable type including, but not limited to, plasmid vectors, cosmid vectors, phage vectors, such as lambda phage, viral vectors, such as adenoviral, AAV or baculoviral vectors, or artificial chromosome vectors such as bacterial artificial chromosomes (BAC), yeast artificial chromosomes (YAC), or P1 artificial chromosomes (PAC).

Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Expression vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Suitable vectors have regulatory sequences, such as promoters, enhancers, terminator sequences, and the like as desired and according to a particular host organism (e.g. bacterial cell, yeast cell). Cloning vectors are generally used to engineer and amplify a certain desired DNA fragment and may lack functional sequences needed for expression of the desired DNA fragments. The construction of expression vectors for use in transfecting prokaryotic cells is also well known in the art, and thus can be accomplished via standard techniques (see, for example, Sambrook, et al. Molecular Cloning: A Laboratory Manual, 4^{th} ed., Cold Spring Harbor Press, Plainsview, N.Y. (2012); and Ausubel et al., Current Protocols in Molecular Biology (Supplement 114), John Wiley & Sons, New York (2016), for definitions and terms of the art.

‘Host cells’ can be either prokaryotic or eukaryotic. The cells can be transiently or stably transfected. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. For all standard techniques see, for example, Sambrook et al., Molecular Cloning: A Laboratory Manual, 4^{th} ed., Cold Spring Harbor Press, Plainsview, N.Y. (2012); and Ausubel et al., Current Protocols in Molecular Biology (Supplement 114), John Wiley & Sons, New York (2016). Recombinant host cells, in the present context, are those which have been genetically modified to contain an isolated DNA molecule, nucleic acid molecule or expression construct or vector of the invention. The DNA can be introduced by any means known to the art which are appropriate for the particular type of cell, including without limitation, transformation, lipofection, electroporation or viral mediated transduction. A DNA construct capable of enabling the expression of the chimeric protein of the invention can be easily prepared by the art-known techniques such as cloning, hybridization screening and Polymerase Chain Reaction (PCR). Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (2012), Wu (ed.) (1993) and Ausubel et al. (2016). Representative host cells that may be used with the invention include, but are not limited to, bacterial cells, yeast cells, insect cells, plant cells and animal cells. Bacterial host cells suitable for use with the invention include Escherichia spp. cells, Bacillus spp. cells, Streptomyces spp. cells, Erwinia spp. cells, Klebsiella spp. cells, Serratia spp. cells, Pseudomonas spp. cells, and Salmonella spp. cells. Animal host cells suitable for use with the invention include insect cells and mammalian cells (most particularly derived from Chinese hamster (e.g. CHO), and human cell lines, such as HeLa. Yeast host cells suitable for use with the invention include species within Saccharomyces, Schizosaccharomyces, Kluyveromyces, Pichia (e.g. Pichia pastoris), Hansenula (e.g. Hansenula polymorpha), Yarowia, Schwaniomyces, Schizosaccharomyces, Zygosaccharomyces and the like. Saccharomyces cerevisiae, S. carlsbergensis and K. lactis are the most commonly used yeast hosts, and are convenient fungal hosts. The host cells may be provided in suspension or flask cultures, tissue cultures, organ cultures and the like. Alternatively, the host cells may also be transgenic animals. The terms “protein”, “polypeptide”, “peptide” are interchangeably used further herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. This term also includes posttranslational modifications of the polypeptide, such as glycosylation, phosphorylation and acetylation. Based on the amino acid sequence and the modifications, the atomic or molecular mass or weight of a polypeptide is expressed in (kilo)dalton (kDa). By “recombinant polypeptide” is meant a polypeptide made using recombinant techniques, i.e., through the expression of a recombinant or synthetic polynucleotide. When the chimeric polypeptide or biologically active portion thereof is recombinantly produced, it is also preferably substantially free of culture medium, i.e., culture medium represents less than about 20% , more preferably less than about 10%, and most preferably less than about 5% of the volume of the protein preparation. By “isolated” is meant material that is substantially or essentially free from components that normally accompany it in its native state. For example, an “isolated polypeptide” refers to a polypeptide which has been purified from the molecules which flank it in a naturally-occurring state, e.g., a fusion protein as disclosed herein which has been removed from the molecules present in the production host that are adjacent to said polypeptide. An isolated chimer can be generated by amino acid chemical synthesis or can be generated by recombinant production. The expression “heterologous protein” may mean that the protein is not derived from the same species or strain that is used to display or express the protein.

“Homologue”, “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. A “substitution”, or “mutation” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence, post-translational modifications and/or functional properties (i.e., altered characteristics) when compared to the wild-type gene or gene product. It is noted that naturally occurring mutants can be isolated; these are identified by the fact that they have altered characteristics when compared to the wild-type gene or gene product. Alternatively, a variant may also include synthetic molecules, e.g. a chemokine ligand variant may be similar in structure and/or function to the natural chemokine, but may concern a small molecule, or a synthetic peptide or protein, which is man-made. Said variants with different functional properties may concerns super-agonists, super-antagonists, among other functional differences, as known to the skilled person.

A “protein domain” is a distinct functional and/or structural unit in a protein. Usually a protein domain is responsible for a particular function or interaction, contributing to the overall role of a protein. Domains may exist in a variety of biological contexts, where similar domains can be found in proteins with different functions. Protein secondary structure elements (SSEs) typically spontaneously form as an intermediate before the protein folds into its three dimensional tertiary structure. The two most common secondary structural elements of proteins are alpha helices and beta (β) sheets, though β-turns and omega loops occur as well. A beta barrel is a beta-sheet composed of tandem repeats that twists and coils to form a closed toroidal structure in which the first strand is bonded to the last strand (hydrogen bond). Beta-strands in many beta-barrels are arranged in an antiparallel fashion. Beta sheets consist of beta strands (also β-strand) connected laterally by at least two or three back-bone hydrogen bonds, forming a generally twisted, pleated sheet. A β-strand is a stretch of poly-peptide chain typically 3 to 10 amino acids long with backbone in an extended conformation. A β-turn is a type of non-regular secondary structure in proteins that causes a change in direction of the polypeptide chain. Beta turns (β turns, β-turns, β-bends, tight turns, reverse turns or β-loops (also called loops herein)) are very common motifs in proteins and polypeptides, which mainly serve to connect β-strands.

The term “circular permutation of a protein” or “circularly permutated protein” refers to a protein which has a changed order of amino acids in its amino acid sequence, as compared to the wild type protein sequence, with as a result a protein structure with different connectivity, but overall similar three-dimensional (3D) shape. A circular permutation of a protein is analogous to the mathematical notion of a cyclic permutation, in the sense that the sequence of the first portion of the wild type protein (adjacent to the N-terminus) is related to the sequence of the second portion of the resulting circularly permutated protein (near its C-terminus), as described for instance in Bliven and Prlic (2012). A circular permutation of a protein as compared to its wild protein is obtained through genetic or artificial engineering of the protein sequence, whereby the N- and C-terminus of the wild type protein are ‘connected’ and the protein sequence is interrupted at another site, to create a novel N- and C-terminus of said protein. The circularly permutated scaffold proteins of the invention are the result of a connected N- and C-terminus of the wild type protein sequence, and a cleavage or interrupted sequence at an accessible or exposed site (preferentially a β-turn or loop) of said scaffold protein, whereby the folding of the circularly permutated scaffold protein is retained or similar as compared to the folding of the wild type protein. Said connection of the N- and C-terminus in said circularly permutated scaffold protein may be the result of a peptide bond linkage, or of introducing a peptide linker, or of a deletion of a peptide stretch near the original N- and C-terminus if the wild type protein, followed by a peptide bond or the remaining amino acids.

The term “fused to”, as used herein, and interchangeably used herein as “connected to”, “conjugated to”, “ligated to” refers, in particular, to “genetic fusion”, e.g., by recombinant DNA technology, as well as to “chemical and/or enzymatic conjugation” resulting in a stable covalent link.

The terms “chimeric polypeptide”, “chimeric protein”, “chimer”, “fusion polypeptide”, “fusion protein”, or “non-naturally-occurring protein” are used interchangeably herein and refer to a protein that comprises at least two separate and distinct polypeptide components that may or may not originate from the same protein. The term also refers to a non-naturally occurring molecule, which means that it is man-made. The term “fused to”, and other grammatical equivalents, such as “covalently linked”, “connected”, “attached”, “ligated”, “conjugated” when referring to a chimeric polypeptide (as defined herein) refers to any chemical or recombinant mechanism for linking two or more polypeptide components. The fusion of the two or more polypeptide components may be a direct fusion of the sequences or it may be an indirect fusion, e.g. with intervening amino acid sequences or linker sequences, or chemical linkers. The fusion of two polypeptides or of a cytokine, such as a chemokine, and a scaffold protein, as described herein, may also refer to a non-covalent fusion obtained by chemical linking. For instance, the C-terminus of the B2 β-strand and the N-terminus of the B3 β-strand of the chemokine core domain could both be linked to a chemical unit, which is capable of binding a complementary chemical unit or binding pocket linked or fused to parts or full length (circularly permutated) scaffold protein, at its exposed or accessible sites.

As used herein, the term “protein complex” or “complex” refers to a group of two or more associated macromolecules, whereby at least one of the macromolecules is a protein. A protein complex, as used herein, typically refers to associations of macromolecules that can be formed under physiological conditions. Individual members of a protein complex are linked by non-covalent interactions. A protein complex can be a non-covalent interaction of only proteins, and is then referred to as a protein-protein complex; for instance, a non-covalent interaction of two proteins, of three proteins, of four proteins, etc. More specifically, a complex of the fusion protein and the cytokine receptor, or a complex of the cytokine- or chemokine-comprising ligand protein (such as a fusion protein) and its specifically bound interactor, such as the cytokine or chemokine receptor that is capable of binding to the cytokine or chemokine ligand. The protein complex of the chemokine-based fusion protein, bound by its chemokine receptor-interacting region (its N-terminus) to a chemokine receptor, for which it is known to bind to said chemokine ligand, to the chemokine receptor, will be the complex formed that is used herein. Alternatively, the protein complex of the interleukin-1 type ligand-based fusion protein, bound by its IL-1 receptor may be the complex as used herein. For instance, it is used in 3D structural analysis, wherein it is the aim to resolve the structure of and interaction between the cytokine ligand receptor and the cytokine interaction site that is part of the fusion protein. More specifically, the interaction or binding site of the chemokine and the chemokine receptor is structurally analysed therein. It is less relevant whether the full structure of the fusion protein is determined. It will be understood that a protein complex can be multimeric. Protein complex assembly can result in the formation of homo-multimeric or hetero-multimeric complexes. Moreover, interactions can be stable or transient. The term “multimer(s)”, “multimeric complex”, or “multimeric protein(s)” comprises a plurality of identical or heterologous polypeptide monomers.

As used herein, the terms “determining,” “measuring,” “assessing,” and “assaying” are used interchangeably and include both quantitative and qualitative determinations.

The terms “suitable conditions” refers to the environmental factors, such as temperature, movement, other components, and/or “buffer condition(s)” among others, wherein “buffer conditions” refers specifically to the composition of the solution in which the assay is performed. The said composition includes buffered solutions and/or solutes such as pH buffering substances, water, saline, physiological salt solutions, glycerol, preservatives, etc. for which a person skilled in the art is aware of the suitability to obtain optimal assay performance.

“Binding” means any interaction, be it direct or indirect. A direct interaction implies a contact between the binding partners. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. In general, a binding domain can be immunoglobulin-based or immunoglobulin-like or it can be based on domains present in proteins, including but not limited to microbial proteins, protease inhibitors, toxins, fibronectin, lipocalins, single chain antiparallel coiled coil proteins or repeat motif proteins. Binding also includes the interaction between a ligand and its receptor, as for the chemokine and chemokine receptor interactions. By the term “specifically binds,” as used herein is meant a binding domain which recognizes a specific target, but does not substantially recognize or bind other molecules in a sample. For a chemokine, it is known to be a ligand for specifically binding a chemokine receptor, so the binding to its receptor is specific. However, in many cases, the chemokines of one subfamily can bind receptors of the same family, so specific binding does not exclude binding to another chemokine receptor. Hence, specific binding does not mean exclusive binding. However, specific binding does mean that such ligands or vice versa such receptors, have a certain increased affinity or preference for one or a few chemokine receptors or vice versa ligands. The term “affinity”, as used herein, generally refers to the degree to which a ligand (as defined further herein) binds to a target protein so as to shift the equilibrium of target protein and ligand toward the presence of a complex formed by their binding. Thus, for example, where a receptor and a ligand are combined in relatively equal concentration, a ligand of high affinity will bind to the receptor so as to shift the equilibrium toward high concentration of the resulting complex.

Methods of determining the spatial conformation of amino acids are known in the art, and include, for example, X-ray crystallography and multi-dimensional nuclear magnetic resonance. The term “conformation” or “conformational state” of a protein refers generally to the range of structures that a protein may adopt at any instant in time. One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., α-helix, β-sheet, β-barrel, among others), tertiary structure (e.g., the three dimensional folding of a polypeptide chain), and quaternary structure (e.g., interactions of a polypeptide chain with other protein subunits). Posttranslational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein may be determined by either functional assay for activity or binding to another molecule or by means of physical methods such as X-ray crystallography, NMR, or spin labeling, among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

Finally, the term “functional fusion protein” or “conformation-selective fusion protein” in the context of the present invention refers to a fusion protein that is functional in binding to its cytokine, or in particular interleukin- or chemokine-receptor protein, optionally in a conformation-selective manner, and/or is functional in activation/inactivation of this receptor (depending on the known features of the ligand: agonist, antagonist, inverse agonist). A binding domain that selectively binds to a particular conformation of a target protein refers to a binding domain that binds with a higher affinity to a target in a subset of conformations than to other conformations that the target may assume. One of skill in the art will recognize that binding domains that selectively bind to a particular conformation of a target will stabilize or retain the target in this particular conformation. For example, an active state conformation-selective binding domain will preferentially bind to a target in an active conformational state and will not or to a lesser degree bind to a target in an inactive conformational state, and will thus have a higher affinity for said active conformational state; or vice versa. The terms “specifically bind”, “selectively bind”, “preferentially bind”, and grammatical equivalents thereof, are used interchangeably herein. The terms “conformational specific” or “conformational selective” are also used interchangeably herein.

### DETAILED DESCRIPTION

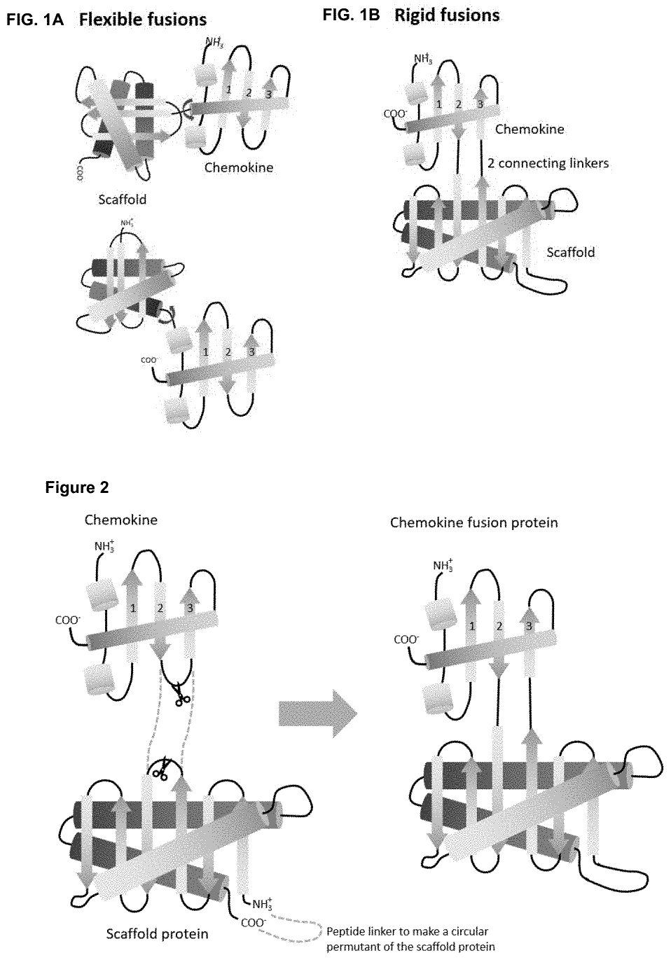

A novel concept for the design of rigidly fused cytokine-containing functional fusion proteins is presented herein. The novel fusion proteins originate through generation of fusions between a cytokine and a scaffold protein, wherein the scaffold protein is a folded protein that interrupts the topology of the cytokine in such a manner that said cytokine still appears in its typical fold and functions to specifically bind its cognate receptor, in a similar manner as compared to the non-fused cytokine ligand. The novel fusion proteins are demonstrated herein as fusions originating from cytokines with a conserved secondary β-strand-based core domain or motif, such as the chemokine cytokines or the interleukin (IL)-1 family. Interruption of said ‘β-strand core domain-containing’ or ‘β-strand-containing domain’ cytokines, as used interchangeably herein, their amino acid sequence by insertion of a scaffold protein, results in an altered topology of the cytokine protein, which though surprisingly still appears in its typical fold and functions to specifically bind its receptor, in a similar manner as compared to the non-fused cytokine ligand. A classical junction of polypeptide components, while typically unjoined in their native state, is performed by joining their respective amino (N-) and carboxyl (C-) termini directly or through a peptide linkage to form a single continuous polypeptide. These fusions are often made via flexible linkers, or at least connected in a flexible manner, which means that the fusion partners are not in a stable position or conformation with respect to each other. As presented in FIG. 1A, by linking proteins via the N- and C-terminal ends, a simple linear concatenation, the fusion is easy, but may be non-stable, prone to degradation, and in some case therefore resulting in non-functional ligand protein. On the other hand, a rigid chimeric/fusion protein as presented herein, with one or more fusion points or connections within the primary topology of two or more proteins, possesses at least one non-flexible fusion point (FIG. 1B). The invention inherently comprises a cytokine ligand protein wherein rotation or bending of the cytokine protein opposed to its fusion partner, the scaffold protein, is prohibited via the creation of several fusions. Through the presence of several fusions within the same chimer, an improved rigidity of the novel chimer of the invention is obtained, and is the result of perfectly designing the fusion sites to allow a fusion that can still retain its cytokine domain folding, as well as its function to bind its receptor. The rigidity of a protein is in fact inherent to the (tertiary) structure of the protein, in this case the novel chimera. It has been shown that increased rigidity can be obtained by altering topologies of known protein folds (King et al., 2015). The rigidity of the fusion created in the fusion protein of the invention hence provides for a rigidity sufficiently strong to ‘orient’ or ‘fix’ the cytokine receptor where the fused cytokine ligand specifically binds to, though mostly the rigidity will still be lower than the rigidity of the target or antigen itself. The fact that the rigid fusion protein of the present invention still maintains its receptor binding and activation functionality, is however a surprising observation, since an interruption of the primary topology, could have resulted in a change in domain or protein folding, impacting tertiary topology and receptor-binding or activation. Although a skilled person is in the capacity to use structural information for designing such a fusion, the actual folding of the fusion protein, which is translated from a novel nucleic acid construct exogenously introduced in a cell, is still unpredictable. It has been demonstrated herein that this interruption of primary topology did not affect receptor binding or activation, leading to the opening of new avenues in the fields involving cytokine receptor structural biology and drug discovery, as shown herein specifically in the field of chemokine and IL receptors. The present invention relates to a novel combination of providing unique next-generation fusion technology, and high affinity and/or conformation-selective chemokine/IL-receptor-binding potential, to allow non-covalent binding of proteins. This novel type of fusion proteins aid in several valuable applications depending on the type of cytokine family, such as chemokine or chemokine variant, and IL or IL-1 receptor type interleukins, or the type of scaffold protein that is used for the generation of the fusion protein. The advantages are numerous, with a straightforward use in structural biology, to facilitate Cryo-EM and X-ray crystallography, for intractable proteins such as the 7 transmembrane proteins as GPCRs. By using this next-generation fusion technology, a leap forward can be foreseen in structural biology of GPCRs and IL-receptor complexes, as rigid chaperone tools are now available and at full implementation also to use those tools to develop improved, more firm therapeutic and diagnostic molecules, such as by structure-based drug design and structure-based screening of novel compounds.

In fact, when used in conformation-selective recognition of cytokine receptors, these tools are applicable as well in binding modes that stabilize the receptor in a functional conformation, such as an active conformation, more specifically an agonist, partial agonist or biased agonist conformation. Depending on the cytokine ligand or ligand variant, further applications of the fusion proteins of the invention are found based on the specific cytokine (chemokine or IL) ligands described to specifically stabilize druggable signaling conformations to enable screening for pathway-selective agonists. With the rapid advancement of such technologies in biotechnology, it is foreseeable that the invention will impact the creation of novel protein therapeutics and in improved performance of current protein drugs.

In a first aspect, the invention relates to a functional fusion protein comprising a cytokine that is fused with a scaffold protein, wherein said scaffold protein is connected to the cytokine protein so that it interrupts the topology of said cytokine via a fusion at least one or more amino acid sites accessible in said cytokine structural fold. Said fusion protein is ‘functional’ in that it retains its receptor-binding functionality in a similar manner as compared to the cytokine ligand not fused to said scaffold protein, in its natural or wild type form. In one embodiment, said fusion protein is a conformation-selective binding domain. The cytokines comprise very diverse superfamilies of ligands, with as preferred cytokine superfamilies those with a β-strand-based or β-strand-containing conserved core domain or motif, revealing accessible amino acid sites at their exposed regions present in β-turns or loops that interconnect these β-strands. The novel fusions should comprise accessible sites far enough from the receptor binding sites of the cytokine, as not to disturb the receptor binding to retain its functionality. The fact that cytokines are relatively small proteins adds a layer of complexity to design such functional fusions, and therefore provides for a surprising solution as presented herein, enabling the skilled person to derive the accessible sites present at exposed turns of these β-strand-based cytokine conserved core domains.

In a first embodiment, the invention relates to a fusion protein comprising a cytokine belonging to the chemokine superfamily, that is fused with a scaffold protein, wherein said scaffold protein is a folded protein of at least 50 amino acids, and is connected to the chemokine core domain so that it interrupts the topology of said core domain via a fusion at at least one or more amino acid sites accessible in said chemokine core domain fold its exposed β-turns. Said fusion protein is further characterized in that it retains its receptor-binding functionality in a similar manner as compared to the chemokine not fused to said scaffold protein, in its natural or wild type form. So, in one embodiment, said fusion protein is a conformation-selective binding domain.

Chemokine protein ligands have been classified according to the characteristic pattern of cysteine residues in proximity to the N-terminus of the mature protein into four subfamilies, CC, CXC, C, and CX3C, wherein X is any amino acid. The basic tertiary structure or architecture of all chemokines however contains a disordered N-terminal ‘signaling domain’ followed by a structured ‘core domain’, which contains an N-loop, a three-stranded β-sheet, and a C-terminal helix (FIG. 2).

Within each subfamily, many chemokines bind multiple receptors and several receptors bind many chemokines. Chemokines are known to dimerize, and different dimerization motifs between different subfamilies were initially supposed to define receptor specificity. However, the functional assays demonstrated that in fact the monomers bind and activate the receptors, while oligomerization seems to be critical for binding to glucosaminoglycans rather. Generally, the chemokine core domain forms the interaction site or chemokine recognition site 1 (CRS1) with the N-terminus of the chemokine receptor, while the N-terminus of the chemokine interacts with the receptor-ligand binding pocket of the receptor (chemokine recognition site 2, CRS2). The first interaction is the binding of the receptor N-terminus to the chemokine core domain (CRS1), allowing to correctly position the chemokine N-terminal signaling domain to enable its interactions with the CRS2 TM pocket. A number of structural studies have shown that receptor binding and activation can at least partially be decoupled. However, further high-resolution structural analysis is required of conformation-specific complexes with intact receptors. Historically, this has been extremely challenging due to the nature of the transmembrane receptors and therefore the limitation to analysis of the more tractable soluble complexes, in most cases using NMR approaches.

A structural role for sulfotyrosines in the receptors has been established, which allows salt bridge formation with homologous basic residues in the β2-β3 hairpin or loop of the chemokine. The chemokine interface with the receptor is believed to involve the N-loop and the β2-β3 strands of the β-sheet of the core domain. Though the fact that structural rearrangements upon CRS1 binding are different from complex to complex, prohibits a simplification of the recognition and activation mechanisms, emphasizing the point for a need for better structural determination tools. Indeed, a number of modified chemokines have also been applied to unravel the role of specific receptors in disease, indicating that ligand pharmacology within the field of cytokines and more particular chemokines would benefit from subtle manipulations that retain high affinity for the receptors, but result in adapted functional outcomes, such as agonistic, inverse agonistic, antagonistic, or super-agonist/antagonistic features. In fact, a general prototype chaperone, such as the fusion protein presented herein, provides for a solution to profile the chemokine ligand/receptor interaction and activation mechanistic features. Chaperone proteins such as nanobodies are known to aid in stabilization of membrane receptor conformations (Manglik et al., 2017), though these types of chaperones do not allow to force the receptor into a conformation wherein the receptor is solely bound to a certain ligand, in a certain conformation. Moreover, the novel chemokine fusion proteins may also provide advantages in drug screening for certain receptor conformational states of intact receptors. So far very few chemokine/receptor complex structures have been determined using intact receptors (CXCR4/vMIP-II, US28/CX3CL1), and more recently the CCR5 receptor with protein inhibitors such as 5P7-CCL5, providing new insights in chemokine-receptor signaling leading to HIV inhibition. The latter has demonstrated that the ligand 5P7-CCL5 interacted with CCR5 in a manner that was not exactly predicted from the two-site model, as described here above, since 5P7-CCL5 its N-loop, β1-strand and 30s-loop were the main interaction sites with the receptor. Previously, more structural data have been obtained using for instance N-terminal peptides of receptors together with a ligand (CXCL8/CXCR1 peptide; CXCL12/CXCR4 sulfopeptide, CCL11/CCR3 peptide), with the risk of only obtaining a partial view on the natural context of the structure.

Another embodiment relates to the novel fusion protein wherein said cytokine is an Interleukin, wherein said scaffold protein interrupts the topology of the interleukin β-barrel core motif at one or more accessible sites in an exposed β-turn of said β-barrel core motif. More specifically, the fusion protein wherein said cytokine is an IL-1 receptor interleukin. The interleukin 1 (IL-1) superfamily of cytokines are important regulators of innate and adaptive immunity, playing key roles in host defense against infection, inflammation, injury, and stress. The ‘IL-1 receptor type interleukin’ superfamily or ‘IL-1 family’ interleukins, as used interchangeably herein, comprises the interleukins IL-1, IL-1α, IL-1β, IL18BP, IL1F5, IL1F6, IL1F7, IL1F8, IL1F10,IL-33, and IL-36, IL36B, and IL-37. These cytokines are related to each other by origin, receptor structure, and signal transduction pathways. The receptors for IL-1 superfamily interleukins share a similar architecture, comprised of three Ig-like domains in their ectodomains, and an intracellular Toll/IL-1R (TIR) domain that is also found among Toll-like receptors. The initiation of cytokine signaling requires two receptors, a primary specific receptor and an accessory receptor that can be shared in some cases. The primary receptor is responsible for specific cytokine binding, while the accessory receptor by itself does not bind the cytokine but associates with the preassembled binary complexes from the cytokine and the primary receptor. The binding of the cytokines to their respective receptors results in a signaling ternary complex, leading to the dimerization of the TIR domains of the two receptors. This initiates intracellular signaling by activating mitogen-activated protein kinases (MAPK) and nuclear factor kappa-light-chain-enhancer of activated B cells (NF-κB). The signaling induces inflammatory responses such as the induction of cyclooxygenase Type 2, increased expression of adhesion molecules, and synthesis of nitric oxide.

The three-dimensional structures of several interleukin cytokines of the IL-1 superfamily have been determined, and demonstrate that despite having limited sequence similarity, these cytokines adopt a conserved signature β-trefoil fold comprised of 12 anti-parallel β-strands that are arranged in a three-fold symmetric pattern. The β-barrel core motif is packed by various amounts of helices in each cytokine structure. Superimposition of the Cα atoms of each of the human cytokines reveals a conserved hydrophobic core, with significant flexibility in the loop regions. Surface residues and loops between β-strands do not appear to be crucial for overall stability and have diverged significantly between the cytokines, consistent with their low sequence similarity and partially explaining their unique recognition by their respective receptors (involving specific loops). For example, human IL-18 shares ˜65% sequence identity to murine IL-18 while sharing only 15% and 18% identity to human IL-1α and human IL-1β, respectively. Nevertheless, IL-18 shows striking similarity to other IL-1 cytokines in its three-dimensional structure. So this IL-1-like receptor interleukins provide for a second example of a superfamily within the cytokines with a β-strand-based conserved structural core domain that is interconnected by flexible β-turns or loops, of which some are involved in receptor recognition, and others may be involved in connecting to folded scaffold proteins as presented herein to obtain the novel enlarged fusion ligands.

An embodiment provides a cytokine fusion protein wherein the β-strands-based conserved core domain is fused with the scaffold protein in such a manner that the scaffold protein is “interrupting” the core domain its topology. In general, the “topology” of a protein refers to the orientation of regular secondary structures with respect to each other in three-dimensional space. Protein folds are defined mostly by the polypeptide chain topology (Orengo et al., 1994). So at the most fundamental level, the ‘primary topology’ is defined as the sequence of secondary structure elements (SSEs), which is responsible for protein fold recognition motifs, and hence secondary and tertiary protein/domain folding. So in terms of protein structure, the true or primary topology is the sequence of SSEs, i.e. if one imagines of being able to hold the N- and C-terminal ends of a protein chain, and pull it out straight, the topology does not change whatever the protein fold. The protein fold is then described as the tertiary topology, in analogy with the primary and tertiary structure of a protein (also see Martin, 2000).

Specifically, as presented herein, the chemokine core domain of the chemokine functional fusion protein of the invention is hence interrupted in its primary topology, by introducing the scaffold protein fusion at an accessible site of an exposed β-turn or loop, between β2 and β3 β-strands of the chemokine core domain, which allowed to retain its 3D-folding and unexpectedly said chemokine also retained its tertiary structure allowing to retain its functional receptor binding capacity. Similarly, the IL-1-like receptor interleukin IL-1β has a conserved β-barrel core motif from which the primary topology is interrupted at an exposed β-turn between 2 β-strands of the conserved core by insertion of a folded scaffold protein as presented herein, with strikingly a retained binding capacity providing for a correctly folded or functional fusion protein.

The “scaffold protein” refers to any type of protein which has a structure or fold allowing a fusion with another protein, in particular with a cytokine or chemokine, as described herein. The classic principle of protein folding is that all the information required for a protein to adopt the correct three-dimensional conformation is provided by its amino acid sequence, resulting in specific folded proteins held together by various molecular interactions. To be useful as a scaffold herein, the scaffold protein must fold into distinct three-dimensional conformations. So, said scaffold protein is defined herein as a ‘folded’ protein, limiting their amino acid length to a minimum, because for short peptides it is generally known that these are very flexible, and not providing for a folded structure. So, the scaffold protein as used in the novel functional fusion proteins used herein are inherently different from peptides or very small polypeptides, such as those composed of 40 amino acids or less, are not considered suitable scaffold proteins for fusing as a Megakine. So, the ‘scaffold protein’ as defined herein is a folded protein of at least 200 amino acids, or 150 amino acids, or at least 100 amino acids, or at least 50 amino acids, or more preferably at least 40 amino acids, at least 30 amino acids, at least 20 amino acids, at least 10 amino acids, at least 9 amino acids. Linkers or peptides, specifically linker of 8 or fewer amino acids are not suited as scaffold proteins for the purpose of the invention. Furthermore, such a “scaffold”, “junction” or “fusion partner” protein preferably has at least one exposed region in its tertiary structure to provide at least one accessible site to cleave as fusion point for the cytokine or chemokine. The scaffold polypeptide is used to assemble with the cytokine or chemokine core domain and thereby results in the fusion protein in a docked configuration to increase mass, provide symmetry, and/or provide an enlarged ligand inducing a specific conformation state of the equivalent receptor and/or improve or add a functionality to the receptor. So, depending on the type of scaffold protein that is used, a different purpose of the resulting fusion protein is foreseen. The type and nature of the scaffold protein is irrelevant in that it can be any protein, and depending on its structure, size, function, or presence, the scaffold protein fused with said cytokine or chemokine core domain as in the fusion protein of the invention will be of use in different application fields. The structure of the scaffold protein will impact the final chimeric structure, so a person skilled in the art should implement the known structural information on the scaffold protein and take into account reasonable expectations when selecting the scaffold. Examples of scaffold proteins are provided in the Examples of the present application, and a non-limiting number of folded proteins that are enzymes, membrane proteins, receptors, adaptor proteins, chaperones, transcription factors, nuclear proteins, antigen-binding proteins themselves, such as Nanobodies, among others, may be applied as scaffold protein to create fusion proteins of the invention. In a preferred embodiment, the 3D-structure of said folded scaffold proteins is known or can be predicted by a skilled person, so the accessible sites to fuse the cytokine or its conserved core domain with can be determined by said skilled person.

The novel chimeric or fusion proteins are fused in a unique manner to avoid that the junction is a flexible, loose, weak link/region within the chimeric protein structure. A convenient means for linking or fusing two polypeptides is by expressing them as a fusion protein from a recombinant nucleic acid molecule, which comprises a first polynucleotide encoding a first polypeptide operably linked to a second polynucleotide encoding the second polypeptide, in the classical known manner. In the recombinant nucleic acid molecule of the present invention however, the interruption of the topology of the cytokine or its conserved core domain by said scaffold is also reflected in the design of the genetic fusion from which said fusion protein is expressed. So, in one embodiment, the fusion protein is encoded by a chimeric gene formed by recombining parts of a gene encoding for a cytokine or specifically chemokine or IL, and parts of a gene encoding the scaffold protein, wherein said encoded scaffold protein interrupts the primary topology of the encoded cytokine, or specifically said chemokine or IL conserved core domain at one or more accessible sites of said domain in its exposed β-turn(s) via at least two or more direct fusions or fusions made by encoded peptide linkers. So, the polynucleotides encoding the polypeptides to be fused are fragmented and recombined in such a way to provide the fusion protein that provides a rigid non-flexible link, connection or fusion between said proteins. The novel chimera are made by fusing the scaffold protein with the cytokine or specifically the conserved chemokine or IL core domain, in such a manner that the primary topology of the cytokine or conserved core domain is interrupted, meaning that the amino acid sequence of the cytokine core domain is interrupted at accessible site(s) and joined to the accessible amino acid(s) of the scaffold protein, which sequence is therefore also possibly interrupted. The junctions are made intramolecularly, in other words internally within the amino acid sequences (see Examples and Figures). So, the recombinant fusions of the present invention result in chimera not solely fused at N- or C-termini, but comprising at least one internal fusion site, where the sites are fused directly or fused via a linker peptide. Where a circularly permutated scaffold is applied to produce the fusion protein, the amino acid sequence of said scaffold protein will be changed by connecting the N- and C-terminus, followed by a cleavage or separation of the amino acid sequence at another site within the sequence of the scaffold protein, corresponding to an accessible site in its tertiary structure, to be fused to the amino acid sequence of the cytokine or chemokine/IL parts. Said N- and C-terminus connection for obtaining the circular permutation may be through a direct fusion, a linker peptide, or even via a short deletion of the region near N- and C-terminus followed by peptide bond of the ends.

The term “accessible site(s)”, “fusion site(s)” or “fusion point” or “connection site” or “exposed site”, are used interchangeably herein and all refer to amino acid sites of the protein sequence that are structurally accessible, preferably positions at the surface of the protein, or exposed to the surface, more preferably exposed regions of β-turns or loops. A person skilled in the art will be able to derive those sites for cytokines from the disclosure as provided herein. The receptor-binding or activation sites of cytokines such as chemokines or ILs often concern such exposed regions, such as for instance the disordered N-terminal signalling domain or the N-loop of the chemokines, or the β-turn between β-strand 4 and β-strand 5 of IL1. However, the interruption of those sites for fusing the chemokine to the scaffold protein may lead to loss of receptor-binding or activation capacity, which is not suitable for the fusion proteins of the invention, and hence not intended to be applied here as accessible fusion site. So, with ‘accessible sites’ and ‘exposed regions’ as ‘loops’ or ‘beta turns’ as described herein is meant those sites and regions that are not the receptor sites or regions, or which may not disturb the receptor binding sites (e.g. sterically). Said binding sites may differ in respect of the targeted receptor, but will generally involve the N-terminal signalling domain and the N-loop of chemokines and the corresponding β-turn between β4 and β5 of IL-1 type receptor interleukins. The N-terminus or C-terminus of the protein is in most cases also a “loose” end of the protein 3D-structure, and therefore accessible from the surface. These can be considered as an accessible site in the chimera of the invention, unless receptor binding or activation requires such ends to be free, and on the condition that at least one other accessible site in the cytokine/chemokine core domain is used for fusion, which leads to an interruption/insertion at that accessible site, interrupting the topology, as this latter accessible site fusion will provide rigidity to the novel chimer. So, accessible sites can therefore include amino- and/or carboxy-terminal sites of the proteins, but the chimer cannot be exclusively based on fusion from accessible sites made up of N- or C-termini. At least one or more sites of the chemokine/IL core domain are used for fusion to the scaffold protein as to result in an interruption of the topology of the known conventional domain fold. So, in one embodiment the at least one accessible site is not an N-terminal and/or C-terminal site of said domain if the at least one is one, and/or does not include an N- or C-terminal site of said domain. In a particular embodiment, the at least one site is not an N- or C-terminal amino acid of said domain. In another embodiment, the accessible site can be an N- or C-terminal site of the conserved core domain, when at least more than one site is used to be fused to the scaffold protein. The scaffold protein is fused via accessible sites visible from its tertiary structure as well, for which in one embodiment, said at least one site is not an N- or C-terminal end of the scaffold protein, and in an alternative embodiment, the at least one site is the N- or C-terminal end of said folded scaffold.

In some embodiments, the fusion protein comprises the N-terminal fragment of said scaffold protein fused at an interruption in an exposed region of said conserved core domain, and the C-terminal fragment of said scaffold protein fused to the C-terminal end near said conserved core.

In some embodiments of the invention, the fusions can be direct fusions, or fusions made by a linker peptide, said fusion sites being immaculately designed to result in a rigid, non-flexible fusion protein. In addition to the position of the selected accessible site(s), the length and type of the linker peptide contributes to the rigidity and possibly the functionality of the resulting fusion protein. Within the context of the present invention, the polypeptides constituting the fusion protein are fused to each other directly, by connection via a peptide bond, or indirectly, whereby indirect coupling assembles two polypeptides through connection via a short peptide linker. Preferred “linker molecules”, “linkers”, or “short polypeptide linkers” are peptides with a length of maximum ten amino acids, more likely four amino acids, typically is only three amino acids in length, but is preferably only two or even more preferred only a single amino acid to provide the desired rigidity to the junction of fusion at the accessible sites. Non-limiting examples of suitable linker sequences are described in the Example section, which can be randomized, and wherein linkers have been successfully selected to keep a fixed distance between the structural domains, as well as to maintain the fusion partners their independent functions (e.g. receptor-binding). In the embodiment relating to the use of rigid linkers, these are generally known to exhibit a unique conformation by adopting a-helical structures or by containing multiple proline residues. Under many circumstances, they separate the functional domains more efficiently than flexible linkers, which may as well be suitable, preferably in a short length of only 1-4 amino acids.

In an alternative embodiment, a fusion protein is described as a rigid fusion protein comprising i) the N-terminal amino acid sequence of cytokine (such as chemokine or IL), ii) a functional scaffold protein, and iii) a cytokine (such as a chemokine or IL) sequence lacking said N-terminal amino acid sequence of i), wherein i) and iii) are concatenated to said scaffold protein of ii). In a preferred embodiment, said rigid fusion protein comprises a N-terminal amino acid sequence which corresponds to the chemokine N-terminal signalling domain, followed by part of the chemokine core domain containing the first two β-strands of the β-sheet, fused to the amino acid sequence of a scaffold protein or a circularly permutated scaffold protein, which is interrupted in its sequence and fused at the accessible sites that correspond to a site in an exposed surface loop or turn, finally fused to the remaining part of the chemokine, which contains the β3 strand of the core domain, and the C-terminal helix of said domain. So the insertion of the scaffold protein into the chemokine protein sequence is obtained at one interrupted amino acid sequence site, corresponding to an accessible site in its β2-β3 turn or loop of the chemokine core domain, which is also called the 40s-loop within the structural terminology of chemokines.

In one embodiment, the accessible site(s) of the chemokine core domain are in an exposed region of the domain fold. Said exposed regions are identified as less fixed amino acid stretches, that are mostly located at the surface of the protein, and on the edges of a structure. Preferably, exposed regions are present as loops or β turns of a protein structure. The most straightforward identification of “exposed regions” of the chemokine core domain are the exposed loops, preferably the β-turns, which are exposed loops located at the edges of the β sheet 3D-structure. For a three-stranded β-sheet structure, the possibilities comprise the β1-β2 turn or loop, also called the 30s loop, or the β2-β3 turn or loop, also called the 40s-loop. In certain chemokine receptor complexes, the 30s-loop is known to involve the receptor binding, and is therefore less preferred for interrupting upon fusion of the scaffold, as compared to the 40s-loop.

In another embodiment, the scaffold protein has a circular permutation. In a preferred embodiment, said circular permutation of the scaffold protein is present at the N- and/or C-terminus of the scaffold protein, or most preferably is between the N- and C-terminus of the scaffold protein. Another embodiment provides a scaffold protein comprising at least two anti-parallel β-strands.

In one embodiment, a fusion protein (with two peptide bonds or two short linkers) is obtained connecting the cytokine or chemokine core domain to the scaffold, via interruption of the cytokine or chemokine core domain primary topology at a cleaved accessible site in its sequence corresponding to the β2-β3 turn, through fusion with a circularly permutated scaffold protein at its cleaved accessible site in its sequence corresponding to an exposed region of its structure (wherein said exposed or accessible site is not N- or C-terminal). So, in the particular embodiment wherein the circular permutation of the scaffold protein is at the N- and C-terminus (as in FIG. 2), the scaffold protein sequence can be recombinantly fused with the cytokine or chemokine fragments as a whole (as in FIG. 7). In a particular embodiment, said fusion protein has its rigidity increased through the additional generation of a strengthening disulfide bridge formed by cysteine residues located within the cytokine or chemokine, preferably near the accessible N-terminal end.

A further aspect of the invention relates to a novel functional fusion protein comprising a cytokine, such as a cytokine comprising a chemokine or IL core domain, fused with a scaffold protein, wherein said scaffold protein interrupts the topology of said cytokine chemokine/IL conserved core domain, and wherein the total mass or molecular weight of the scaffold protein(s) is at least 30 kDa, so that the addition of mass and structural features by binding of the fusion to the target, such as the receptor of the ligand, will be significant and sufficient to allow 3-dimensional structural analysis of the target when non-covalently bound to said chimer. In another embodiment, the total mass or molecular weight of the scaffold protein(s) is at least 40, at least 45, at least 50, or at least 60 kDa. This particular size or mass increase will affect the signal-to-noise ratio in the images to decrease. Secondly, the chimer will offer a structural guide by providing adequate features for accurate image alignment for small or difficult to crystallize proteins to reach a sufficiently high resolution using cryo-EM and X-ray crystallography.

A further aspect of the invention relates to a nucleic acid molecule encoding said fusion protein of the present invention. Said nucleic acid molecule comprises the coding sequence of said cytokine, chemokine, or interleukin, and said scaffold protein(s), and/or fragments thereof, wherein the interrupted topology of said domain is reflected in the fact that said domain sequence will contain an insertion of the scaffold protein sequence(s) (or a circularly permutated sequence, or a fragment thereof), so that the N-terminal cytokine, chemokine, or IL-fragment and C-terminal cytokine, chemokine, or IL-conserved core domain fragment are separated by the scaffold protein sequence or fragments thereof within said nucleic acid molecule. In another embodiment, a chimeric gene is described with at least a promoter, said nucleic acid molecule encoding the fusion protein, and a 3′ end region containing a transcription termination signal. Another embodiment relates to an expression cassette encoding said fusion protein of the present invention, or comprising the nucleic acid molecule or the chimeric gene encoding said fusion protein. Said expression cassettes are in certain embodiments applied in a generic format as a library, containing a large set of cytokine, such as chemokine or interleukin, fusions to select for the most suitable binders of the receptor or antibody or alternative target or interaction partner(s). Further embodiments relate to vectors comprising said expression cassette or nucleic acid molecule encoding the fusion protein of the invention. In particular embodiments, vectors for expression in E. coli allow to produce the fusion proteins and purify them in the presence or absence of their targets. Alternative embodiments relate to host cells, comprising the fusion protein of the invention, or the nucleic acid molecule or expression cassette or vector encoding the fusion protein of the invention. In particular embodiments, said host cell further co-expresses the target protein or for instance receptor that specifically binds the cytokine, such as a chemokine or IL, of the fusion protein. Another embodiment discloses the use of said host cells, or a membrane preparation isolated thereof, or proteins isolated therefrom, for ligand screening, drug screening, protein capturing and purification, or biophysical studies. The present invention providing said vectors further encompasses the option for high-throughput cloning in a generic fusion vector. Said generic vectors are described in additional embodiments wherein said vectors are specifically suitable for surface display in yeast, phages, bacteria or viruses. Furthermore, said vectors find applications in selection and screening of immune libraries comprising such generic vectors or expression cassettes with a large set of different ligands, in particular with different linkers for instance. So, the differential sequence in said libraries constructed for the screening of novel fusion protein for specific receptors is provided by the difference in the linker sequence, or alternatively in other regions.

In one embodiment, the vectors of the present invention are suitable to use in a method involving displaying a collection of cytokine fusion proteins at the extracellular surface of a population of cells. Surface display methods are reviewed in Hoogenboom, (2005; Nature Biotechnol 23, 1105-16), and include bacterial display, yeast display, (bacterio)phage display. Preferably, the population of cells are yeast cells. The different yeast surface display methods all provide a means of tightly linking each fusion protein encoded by the library to the extracellular surface of the yeast cell which carries the plasmid encoding that protein. Most yeast display methods described to date use the yeast Saccharomyces cerevisiae, but other yeast species, for example, Pichia pastoris, could also be used. More specifically, in some embodiments, the yeast strain is from a genus selected from the group consisting of Saccharomyces, Pichia, Hansenula, Schizosaccharomyces, Kluyveromyces, Yarrowia, and Candida. In some embodiments, the yeast species is selected from the group consisting of S. cerevisiae, P. pastoris, H. polymorpha, S. pombe, K. lactis, Y. lipolytica, and C. albicans. Most yeast expression fusion proteins are based on GPI (Glycosyl-Phosphatidyl-Inositol) anchor proteins which play important roles in the surface expression of cell-surface proteins and are essential for the viability of the yeast. One such protein, alpha-agglutinin consists of a core subunit encoded by AGA1 and is linked through disulfide bridges to a small binding subunit encoded by AGA2. Proteins encoded by the nucleic acid library can be introduced on the N-terminal region of AGA1 or on the C-terminal or N-terminal region of AGA2. Both fusion patterns will result in the display of the polypeptide on the yeast cell surface.

The vectors disclosed herein may also be suited for prokaryotic host cells to surface display the proteins. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformnis 41 P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting. When the host cell is a prokaryotic cell, examples of suitable cell surface proteins include suitable bacterial outer membrane proteins. Such outer membrane proteins include pili and flagella, lipoproteins, ice nucleation proteins, and autotransporters. Exemplary bacterial proteins used for heterologous protein display include LamB (Charbit et al., EMBO J, 5(11): 3029-37 (1986)), OmpA (Freudl, Gene, 82(2): 229-36 (1989)) and intimin (Wentzel et al., J Biol Chem, 274(30): 21037-43, (1999)). Additional exemplary outer membrane proteins include, but are not limited to, FliC, pullulunase, OprF, OprI, PhoE, MisL, and cytolysin. An extensive list of bacterial membrane proteins that have been used for surface display are detailed in Lee et al., Trends Biotechnol, 21(1): 45-52 (2003), Jose, Appl Microbiol Biotechnol, 69(6): 607-14 (2006), and Daugherty, Curr Opin Struct Biol, 17(4): 474-80 (2007).

Furthermore, to allow an in-depth screening selection, vectors can be applied in yeast and/or phage display, followed FACS and panning, respectively. Display of cytokine or chemokine fusion proteins on yeast cells in combination with the resolving power of fluorescent-activated cell sorting (FACS), for instance, provides a preferred method of selection. In yeast display each cytokine or chemokine fusion protein is for instance displayed as a fusion to the Aga2p protein at ˜50.000 copies on the surface of a single cell. For selection by FACS, the labelling with different fluorescent dyes will determine the selection procedure. The fusion protein-displaying yeast library can next be stained with a mixture of the used fluorescent proteins. Two-colour FACS can then be used to analyse the properties of each fusion protein that is displayed on a specific yeast cell to resolve separate populations of cells. Yeast cells displaying a fusion protein that is highly suitable for binding the protein of interest, such as a receptor or antibody, will bind and can be sorted along the diagonal in a two-colour FACS. The use of vectors for such a selection method is most preferred when screening of fusion proteins specifically targeting a transient protein-protein interaction or conformation-selective binding state for instance. Similarly, vectors for phage display are applied, and used for display of the fusion proteins on the bacteriophages, followed by panning. Display can for instance be done on M13 particles by fusion of the cytokine or chemokine fusion proteins, within said generic vector, to phage coat protein III (Hoogenboom, 2000; Immunology today. 5699:371-378). For selection of fusion proteins specifically binding certain conformations and/or a transient protein-protein interaction for instance, only one of the interacting protomers is immobilized onto the solid phase. Bio-selection by panning of the phage-displayed fusion proteins is then performed in the presence of excess amounts of the remaining soluble protomer.

Optionally, one can start with a round of panning on a cross-linked complex or protein that is immobilized on the solid phase.

Another aspect of the invention relates to a complex comprising said fusion protein, and a receptor protein(s), wherein said receptor protein is specifically bound to the cytokine, such a chemokine or interleukin among other types of cytokine and their cognate receptors. More particular, an embodiment relates to a protein complex wherein said receptor protein is bound to the cytokine part of said fusion protein. One embodiment discloses a complex as described herein, wherein the cytokine or chemokine or IL of said fusion protein is a conformation selective ligand. More particularly, a complex is disclosed wherein the cytokine or chemokine or IL part of the fusion protein stabilizes the receptor protein in a functional conformation. More specifically said functional conformation may involve an agonist conformation, may involve a partial agonist conformation, or a biased agonist conformation, among others. Alternatively, a complex of the invention is disclosed, wherein the cytokine or chemokine or IL of the fusion proteins stabilizes the receptor protein in a functional conformation, wherein said functional conformation is an inactive conformation, or wherein said functional conformation involves an inverse agonist conformation. Another embodiment relates to said cytokine fusion protein or chemokine or IL fusion protein in complex with its receptor, wherein the receptor is activated upon binding to the fusion protein. As previously described herein, a number of cytokine receptors, including the chemokine and/or IL receptors, require several interfaces to bind to the ligand to acquire an activated state.

Another embodiment of the invention relates to a method of producing the cytokine functional fusion protein according to the invention comprising the steps of (a) culturing a host comprising the vector, expression cassette, chimeric gene or nucleic acid sequence of the present invention, under conditions conducive to the expression of the fusion protein, and (b) optionally, recovering the expressed polypeptide.

A more specific embodiment relates to a method for producing the chemokine fusion protein as described herein, comprising the steps of: (a) selecting a chemokine ligand and a scaffold protein of which the 3-D structure reveals accessible sites in exposed regions as loops or turns for interruption of the amino acid sequence without interrupting the primary topology, (b) designing a genetic fusion construct wherein the nucleic acid sequence is designed to encode a protein sequence encoded by a nucleic acid sequence molecule in which:
1. an interruption of the chemokine sequence is present at the position corresponding to the accessible site between the β-strand β2 and β-strand β3 of the chemokine protein its conserved core domain structure,
2. the scaffold sequence for insertion by fusing its 5′ and 3′ nucleic acid sequence ends (so as a whole), or the scaffold protein for insertion by fusing alternative interrupted sited of said scaffold protein its sequence present at an accessible site of said scaffold, such as a loop or a β-turn,
3. the most 5′ interrupted sequence 3′end of the chemokine (corresponding to an amino acid residue C-terminally of β-strand β2) is fused to the 5′ start of the most 5′-(interrupted) site of the scaffold protein, and the 5′ start of the most C-terminal interrupted site of the chemokine (corresponding to the amino acid residue N-terminally of β-strand β3) is fused to the 3′ end of the most C-terminally interrupted site of the scaffold protein,
(c) introducing said genetic fusion construct in an expression system to obtain a fusion protein wherein said chemokine is fused at two or more sites of its core domain to the scaffold protein.

An alternative embodiment discloses a method for producing or generating a fusion protein as described herein, comprising the steps of: (a) selecting a chemokine ligand and a scaffold protein with accessible loops or turns in their tertiary structure, which can be interrupted to create a fusion protein without interruption of primary topology of the chemokine and/or of the primary topology of the scaffold protein, (b) designing a genetic fusion construct wherein the nucleic acid sequence is designed as such to code for a protein in an expression host wherein:
1. the protein sequence of the chemokine is interrupted at an amino acid corresponding to an accessible site between the β-strand β2 and β-strand β3 of the core domain,
2. the scaffold protein its N- and C-terminal ends are fused to obtain a circularly permutated scaffold protein,
3. the circularly permutated scaffold protein of 2. is then interrupted in its amino acid sequence corresponding to an accessible site in an exposed loop or turn of its tertiary sequence, which is an interruption site that is different from the amino acids that were fused in step 2.
4. the C-terminal end of the N-terminal part of the chemokine (i.e. the interrupted site of the chemokine C-terminally of β-strand β2) is fused to the N-terminus of the circularly permutated scaffold protein, and the N-terminal start of the C-terminal part of the chemokine (i.e. the interrupted site of the chemokine N-terminally of β-strand β3) is fused to the C-terminus of the circularly permutated scaffold protein,
(c) introducing said genetic fusion construct in an expression system to obtain a fusion protein wherein said chemokine is fused at two or more sites of its core domain to the circularly permutated scaffold protein.

Another aspect relates to the use of the cytokine functional fusion protein of the present invention or of the use of the nucleic acid molecule, chimeric gene, the expression cassette, the vectors, or the complex, in structural analysis of its cognate receptor protein. In particular, the use of the β-strand-core domain based cytokine fusion protein in structural analysis of a receptor protein wherein said receptor protein is a protein specifically bound to said cytokine part of said fusion protein. “Solving the structure” or “structural analysis” as used herein refers to determining the arrangement of atoms or the atomic coordinates of a protein, and is often done by a biophysical method, such as X-ray crystallography or cryogenic electron-microscopy (cryo-EM). Specifically, an embodiment relates to the use in structural analysis comprising single particle cryo-EM or comprising crystallography. The use of such cytokine fusion proteins of the present invention in structural biology renders the major advantage to serve as crystallization aids, namely to play a role as crystal contacts and to increase symmetry, and even more to be applied as rigid tools in cryo-EM, which will be very valuable to solve large structures of intractable proteins such as membrane receptors, to reduce size barriers coped with today, also to increase symmetry, and to stabilize and visualize specific conformational states of the receptor in complex with said cytokine or chemokine fusion protein.

Using cryo-EM for structure determination has several advantages over more traditional approaches such as X-ray crystallography. In particular, cryo-EM places less stringent requirements on the sample to be analysed with regard to purity, homogeneity and quantity. Importantly, cryo-EM can be applied to targets that do not form suitable crystals for structure determination. A suspension of purified or unpurified protein, either alone or in complex with other proteinaceous molecules such as a cytokine fusion protein of the invention or non-proteinaceous molecules such as a nucleic acid, can be applied to carbon grids for imaging by cryo-EM. The coated grids are flash-frozen, usually in liquid ethane, to preserve the particles in the suspension in a frozen-hydrated state. Larger particles can be vitrified by cryofixation. The vitrified sample can be cut in thin sections (typically 40 to 200 nm thick) in a cryo-ultramicrotome, and the sections can be placed on electron microscope grids for imaging. The quality of the data obtained from images can be improved by using parallel illumination and better microscope alignment to obtain resolutions as high as ˜3.3 Å. At such a high resolution, ab initio model building of full-atom structures is possible. However, lower resolution imaging might be sufficient where structural data at atomic resolution on the chosen or a closely related target protein and the selected heterologous protein or a close homologue are available for constrained comparative modelling. To further improve the data quality, the microscope can be carefully aligned to reveal visible contrast transfer function (CTF) rings beyond ⅓ Å⁻¹ in the Fourier transform of carbon film images recorded under the same conditions used for imaging. The defocus values for each micrograph can then be determined using software such as CTFFIND.

Further, a method is disclosed herein for determining a 3-dimensional structure of a ligand/receptor complex comprising the steps of: (i) providing the fusion protein according to the invention, and providing the receptor to form a complex, wherein said receptor protein is bound to the cytokine part of the fusion protein of the invention, or providing the complex as described herein above; (ii) display said complex in suitable conditions for structural analysis, wherein the 3D structure of said protein complex is determined at high-resolution.

In a specific embodiment, said structural analysis is done via X-ray crystallography. In another embodiment, said 3D analysis comprises cryo-EM. More specifically, a methodology for cryo-EM analysis is described here as follows. A sample (e.g. the fusion protein of choice in a complex with a receptor of interest), is applied to a best-performing discharged grid of choice (carbon-coated copper grids, C-Flat, 1.2/1.3 200-mesh: Electron Microscopy Sciences; gold R1.2/1.3 300 mesh UltraAuFoil grids: Quantifoil; etc.) before blotting, and then plunge-frozen in to liquid ethane (Vitrobot Mark IV (FEI) or other plunger of choice). Data for a single grid are collected at 300 kV Electron Microscope (Krios 300 kV as an example with supplemented phase plate of choice) equipped with a detector of choice (Falcon 3EC direct-detector as an example). Micrographs are collected in electron-counting mode at a proper magnification suitable for an expected ligand/receptor complex size. Collected micrographs are manually checked before further image processing. Apply drift correction, beam induced motion, dose-weighting, CTF fitting and phase shift estimation by a software of choice (RELION, SPHIRE packages as examples). Pick particles with a software of choice and use them for to 2D classification. Manually-inspected 2D classes and remove false positives. Bin particles accordingly to data collection settings. Generate an initial 3D reference model by applying a proper low-pass filter and generate a number (six as an example) of 3D classes. Use original particles for 3D refinement (if needed use soft mask). Estimate a reconstruction resolution by using Fourier Shell Correlation (FSC)=0.143 criterion. Local resolution can be calculated by the MonoRes implementation in Scipion. Reconstructed cryo-EM maps can be analyzed using UCSF Chimera and Coot software. The design model can be initially fitted using UCSF Chimera and analyzed by software of choice (UCSF Chimera, PyMOL or Coot).

Another advantage of the method of the invention is that structural analysis, which is in a conventional manner only possible with highly pure protein, is less stringent on purity requirements thanks to the use of the cytokine fusion proteins. Such cytokine ligand fusion proteins, more particular such β-strand conserved core domain-based cytokine fusion proteins such as chemokine or IL-1 fusion proteins, will specifically filter out the receptor of interest via its high affinity binding site, within a complex mixture. The receptor protein can in this way be trapped, frozen and analysed via cryo-EM.

Said method is in alternative embodiments also suitable for 3D analysis wherein the receptor protein is a transient protein-protein complex or is in a transient specific conformational state. Additionally, said fusion protein molecules can also be applied in a method for determining the 3-dimensional structure of a receptor to stabilize transient protein-protein interactions as targets to allow their structural analysis.

Another embodiment relates to a method to select or to screen for a panel of fusion proteins binding to different conformations of the same receptor protein, comprising the steps of: (i) designing a ligand library of fusion proteins binding the receptor protein, and (ii) selecting the fusion proteins via surface yeast display, phage display or bacteriophages to obtain a fusion protein panel comprising proteins binding to several relevant conformational states of said receptor protein, thereby allowing several conformations of the receptor protein to be analysed in for instance cryo-EM in separate images. To obtain specific or certain conformational states, one can make use of cell-based systems wherein the receptor is on the membrane, wherein said cells may be treated or manipulated according to the purpose of the experiment.

In another embodiment, said method and said fusion protein of the invention is used for structure-based drug design and structure-based drug screening. The iterative process of structure-based drug design often proceeds through multiple cycles before an optimized lead goes into phase I clinical trials. The first cycle includes the cloning, purification and structure determination of the receptor protein or nucleic acid by one of three principal methods: X-ray crystallography, NMR, or homology modelling. Using computer algorithms, compounds or fragments of compounds from a database are positioned into a selected region of the structure. One could use the fusion protein of the invention to fix or stabilize certain structural conformations of a receptor. The selected compounds are scored and ranked based on their steric and electrostatic interactions with this target site, and the best compounds are tested with biochemical assays. In the second cycle, structure determination of the target in complex with a promising lead from the first cycle, one with at least micromolar inhibition in vitro, reveals sites on the compound that can be optimized to increase potency. Also at this point, the fusion protein of the invention may come into play, as it facilitates the structural analysis of said target receptor protein in a certain conformational state. Additional cycles include synthesis of the optimized lead, structure determination of the new target:lead complex, and further optimization of the lead compound. After several cycles of the drug design process, the optimized compounds usually show marked improvement in binding and, often, specificity for the target. A library screening leads to hits, to be further developed into leads, for which structural information as well as medicinal chemistry for Structure-Activity-Relationship analysis is essential.

Another embodiment relates to a method of identifying (conformation-selective) compounds, comprising the steps of:
i) providing a target receptor protein and a fusion protein of the invention specifically binding said target receptor protein
ii) providing a test compound
iii) evaluating the selective binding of the test compound to the target receptor protein.

According to a particularly preferred embodiment, the above described method of identifying conformation-selective compounds is performed by a ligand binding assay or competition assay, even more preferably a radioligand binding or competition assay. Most preferably, the above described method of identifying conformation-selective compounds is performed in a comparative assay, more specifically, a comparative ligand competition assay, even more specifically a comparative radioligand competition assay.

It is to be understood that although particular embodiments, specific configurations as well as materials and/or molecules, have been discussed herein for engineered cells and methods according to the disclosure, various changes or modifications in form and detail may be made without departing from the scope of this invention. The following examples are provided to better illustrate particular embodiments, and they should not be considered limiting the application. The application is limited only by the claims.

### EXAMPLES

General

We have designed a novel type functional rigid fusion protein, also called ‘Megakine’ (Mk), consisting of a cytokine and a scaffold protein, wherein the β-strand-based conserved core domain or motif of the cytokine, or a particular subfamily of cytokines, are connected to a scaffold protein via two or three short linkers, or via two or three direct linkages. The principle is exemplified herein for 2 superfamilies of cytokines, comprising the chemokines (specifically by CCL5 and CXCL12), and the interleukins, more specifically the IL-1 type receptor interleukins, both of these superfamilies being representative for such β-strand-based conserved core domain-comprising cytokines. Depending on the mechanism of action and binding mode of the chemokine or interleukin to its receptor, these rigid fusion proteins bind and fix specific and different conformational states of the chemokine- or interleukin-receptor. Those fusion proteins represent enlarged chemokine or interleukin ligands in fact, and are instrumental for determining protein structures of chemokine or interleukin complexes (with their receptors for instance), and aid in several applications including X-ray crystallography and cryo-EM applications. The Megakines function as next generation crystallization chaperones by reducing the conformational flexibility of the bound cognate cytokine receptor and by extending the surfaces predisposed to forming crystal contacts, as well as by providing additional phasing information. By mixing a specific Megakine protein with the chemokine- or interleukin-specific receptor, their specific binding interaction leads to “mass” addition and fixing a specific conformational state of the receptor.

As a proof of concept of this approach, we inserted as a folded scaffold protein a circularly permutated variant (c7HopQ) of the gene encoding the adhesion domain of HopQ (a periplasmic protein from H. pylori, PDB 5LP2) in the β-turn between β-strand 2 (β2) and the β-strand 3 (β3) of the chemokine core domain of the chemokine CCL5 variant 6P4 (a super agonist) FIG. 2 (Example 1) and of the chemokine core domain of the chemokine CXCL12 (FIG. 19) (Example 7). Alternatively, we inserted said c7HopQ scaffold in the β-turn between β-strand 6 (β6) and the β-strand 7 (β7) of the β-barrel core motif or domain of the interleukin IL-1β (FIG. 27) (Example 10). Moreover, for the CCL5 chemokine, an alternative Megakine was generated making use of a larger scaffold protein, E. coli Ygjk (PDB code 3W7S; Kurakava et al, 2008) for which 2 circularly permutated variants (C1Ygjk and C2Ygjk) were designed to test in said Megakine fusions with CCL5 6P4 (Example 8).

Constructs were designed using Modeller Software (https://salilab.org/modeller/), and different fusions were made, with different short linkers.

We performed yeast surface display of several different fusion protein constructs, containing different linkers (Example 6, 8, 10), which demonstrated that all different constructs for the cytokine-based Megakines were capable of binding a cytokine ligand-specific monoclonal antibody (Example 2, 9, and 11). We expressed these fusion proteins as a secreted protein in yeast (Example 3) and in the periplasm of E. coli (Example 4). Moreover, in Example 5 we show that the purified protein or periplasmic extracts applied in cell-based assay are capable of activating the CCR5 receptor, even in some instance to the level that is observed for the 6P4-CCL5 chemokine agonist itself.

### Example 1

### Design and Generation of a 50 kDa Fusion Protein Built from a c7HopQ Scaffold Inserted into the β-strand β2-β3-Connecting β-turn of a 6P4-CCL5 chemokine

As a first proof of concept of obtaining rigid fusion proteins ‘Megakines’, an improved CCL5 chemokine, called 6P4-CCL5 chemokine was grafted onto a large scaffold protein via two peptide bonds that connect 6P4-CCL5 to a scaffold according to FIG. 2 to build a rigid Megakine.

The 50 kDa Megakine described here is a chimeric polypeptide concatenated from parts of chemokine and parts of a scaffold protein connected according to FIGS. 2 to 6. Here, the chemokine used is the 6P4-CCL5, derived from the natural CCL5 ligand, belonging to the subfamily of CC-chemokines, which was modified to a super agonist of CCR5 GPCR as depicted in SEQ ID NO:1 (6P4-CCL5 is an analogue of the antagonist CCL5-5P7; Zheng et al. 2017; PDB code CCL5-5P7: 5UIW). The β-turn connecting β-strand 2 and β-strand 3 of 6P4-CCL5 was interrupted for fusion to the scaffold protein. The scaffold protein is an adhesin domain of Helicobacter pylori strain G27 (PDB: 5LP2; SEQ ID NO:2) called HopQ (Javaheri et al, 2016). The N- and C-terminus of HopQ was connected, although after a truncation of seven amino acids in the circular permutation region (called c7HopQ) which otherwise appeared as a loop never fully visible in electron density of crystal structures. This truncated fusion creates a circularly permutated variant of HopQ, called c7HopQ, wherein a cleavage within the amino acid sequence was made somewhere else in its sequence (i.e. in a position corresponding to an accessible site in an exposed region of said scaffold protein). To design functional Megakine fusion protein variants, in silico molecular modelling using Modeler software was used (https://salilab.org/modeller) as well as custom-written Python scripts. As a result, four low free energy Mk_{6P4-CCL5}^{c7HopQ} models were generated, where all parts were connected to each other from the amino (N-) to the carboxy (C-)terminus in the next given order by peptide bonds:

Mk_{6P4-CCL5}^{cHopQ}V1 (SEQ ID NO: 3): N-terminus until β-strand 2 of the 6P4-CCL5 chemokine (1-43 of SEQ ID NO:1), a C-terminal part of HopQ (residues 193-411 of SEQ ID NO: 2), an N-terminal part of HopQ (residues 18-185 of SEQ ID NO: 2), the C-terminal part from β-strand 3 till end of the 6P4-CCL5 chemokine (47-69 of SEQ ID NO: 1), 6× His tag and EPEA tag (U.S. Pat. No. 9,518,084 B2; SEQ ID NO: 21).

Mk_{6P4-CCL5}^{c7HopQ}V2 (SEQ ID NO: 4): N-terminus until β-strand 2 of the 6P4-CCL5 chemokine (1-44 of SEQ ID NO: 1), Thr one amino acid linker, a C-terminal part of HopQ (residues 194-411 of SEQ ID NO: 2), an N-terminal part of HopQ (residues 18-185 of SEQ ID NO:2), the C-terminal part from β-strand 3 till end of the 6P4-CCL5 chemokine (47-69 of SEQ ID NO: 1), 6× His tag and EPEA tag.

Mk_{6P4-CCL5}^{c7HopQ}V3 (SEQ ID NO:5): N-terminus until β-strand 2 of the 6P4-CCL5 chemokine (1-45 of SEQ ID NO: 1), a C-terminal part of HopQ (residues 192-411 of SEQ ID NO: 2), an N-terminal part of HopQ (residues 18-185 of SEQ ID NO: 2), the C-terminal part from β-strand 3 till end of the 6P4-CCL5 chemokine (47-69 of SEQ ID NO: 1), 6× His tag and EPEA tag (U.S. Pat. No. 9,518,084 B2).

Mk_{6P4-CCL5}^{c7HopQ}V4 (SEQ ID NO: 6): N-terminus until β-strand 2 of the 6P4-CCL5 chemokine (1-44 of SEQ ID NO: 1), a C-terminal part of HopQ (residues 193-411 of SEQ ID NO: 2), an N-terminal part of HopQ (residues 18-185 of SEQ ID NO: 2), the C-terminus from β-strand 3 till end of the 6P4-CCL5 chemokine (47-69 of SEQ ID NO: 1), 6× His tag and EPEA tag.

### Example 2

### Yeast Display of 50 kDa Fusion Proteins Built from a c7HopQ Scaffold Inserted into the β-strand β2-β3-connecting β-turn of a 6P4-CCL5 chemokine

To demonstrate that four Mk_{6P4-CCL5}^{c7HopQ}V1-V4 Megakine variants (SEQ ID NO: 3-6) can be expressed as a well folded and functional protein, we displayed this protein on the surface of yeast (Boder, 1997). Proper folding of 6P4-CCL5 chemokine part was examined by using a fluorescent conjugated monoclonal antibody that binds to functional 6P4-CCL5 chemokine (Alexa Fluor® 647 anti-human RANTES (CCL5) Antibody from Biolegend, ref 515506; anti-CCL5-mAb647). In order to display the Mk_{6P4-CCL5}^{c7HopQ}V1-V4 Megakine variants on yeast, we used standard methods to construct an open reading frames that encodes the Megakine in fusion to a number of accessory peptides and proteins (SEQ ID NO:7-10): the appS4 leader sequence that directs extracellular secretion in yeast (Rakestraw, 2009), Mk_{6P4-CCL5}^{c7HopQ} Megakine variant, a flexible peptide linker, the Aga2p the adhesion subunit of the yeast agglutinin protein Aga2p which attaches to the yeast cell wall through disulfide bonds to the Aga1p protein, an acyl carrier protein for the orthogonal fluorescent staining of the displayed fusion protein (Johnsson, 2005) followed by the cMyc Tag. This open reading frame was put under the transcriptional control of galactose-inducible GAL1/10 promotor into the pCTCON2 vector (Chao, 2006) and introduced into yeast strain EBY100.

EBY100 yeast cells, bearing this plasmid, were grown and induced overnight in a galactose-rich medium to trigger the expression and secretion of the Mk_{6P4-CCL5}^{c7HopQ}-Aga2p-ACP fusion. For the orthogonal staining of ACP, cells were incubated for 1 h in the presence a fluorescently labelled CoA analogue (CoA-547, 2 μM) and catalytic amounts of the SFP synthase (1 μM). To analyse the functionality of the displayed Megakine, we examined its ability to be recognized by Alexa Fluor® 647 fluorescently labelled anti-CCL5 monoclonal antibody (anti-CCL5-mAb647) by flow cytometry. Accordingly, EBY100 yeast cells were induced and fluorescently stained orthogonally with CoA547 to monitor the display of Mk_{6P4-CCL5}^{c7HopQ}-Aga2p-ACP fusions. These orthogonally stained yeast cells were next incubated 1 h in the presence of different concentrations of anti-CCL5-mAb647 (15, 31, 62, 125 and 250 ng/mL). In these experiments, induced yeast cells were washed and subjected to flow-cytometry to measure the Megakine display level of each cell by comparing the CoA547-fluorescence level to yeast cells that display the MegaBody Mb_{Nb207}^{cHopQ}-Aga2p-ACP fusion (SEQ ID NO: 11; wherein a MegaBody is similar to a Megakine, but instead of a chemokine a Nanobody (Nb) is fused to a scaffold protein, with herein Nb₂₀₇ as a GFP-specific Nb) and were stained orthogonally in the same way. Next, the binding of anti-CCL5-mAb647 was analyzed by examination of 647-fluorescence level that should be linearly correlated to expression level of Mb_{Nb207}^{cHopQ} on the surface of yeast. Indeed, a two-dimensional flow cytometric analysis confirmed that anti-CCL5-mAb647 (high 647-fluorescence level) only binds to yeast cells with significant Megakine display levels (high CoA547-fluorescence level) (FIG. 9 and FIGS. 10-14). In contrast, anti-CCL5-mAb647 does not bind to yeast cells that display Mega Body Mb_{Nb207}^{cHopQ}-Aga2p-ACP fusion (SEQ ID NO: 11) and have been stained in the same way.

We conclude from these experiments that all four Mk_{6P4}^{c7HopQ}V1-V4 Megakine variants (SEQ ID NO: 3-6) can be expressed as a well folded and functional chimeric protein on the surface of yeast.

### Example 3

### Yeast Expression and Purification of 50 kDa Fusion Proteins Built from a c7HopQ Scaffold Inserted into the β-dtrand β2-β3-connecting β-turn of a 6P4-CCL5 chemokine

As we were able to display a functional Megakine on the surface of yeast, we set out to express these 50 kDa fusion proteins in the EBY100 cells as soluble secreted proteins, purified them to homogeneity and determined their properties.

In order to express four Megakines Mk_{6P4-CCL5}^{c7HopQ}V1-V4 Megakine variants (SEQ ID NO: 3-6) we used standard methods to construct open reading frames that encode the Megakine in fusion to a number of accessory peptides and proteins (SEQ ID NO:12-15): the appS4 leader sequence that directs extracellular secretion in yeast (Rakestraw, 2009), Mk_{6P4-CCL5}^{c7HopQ} Megakine variant, 6× His tag, EPEA tag and STOP codon that finish the translation. This open reading frame was put under the transcriptional control of galactose-inducible GAL1/10 promotor into the pCTCON2 vector (Chao, 2006) and introduced into yeast strain EBY100. EBY100 yeast cells, bearing this plasmid, were grown and induced overnight in a galactose-rich medium to trigger the expression and secretion of the Mk_{6P4}^{c7HopQ}V1-V4 variants (SEQ ID NO:12-15) at 30° C. Recombinant Megakine fusion proteins were recovered from the medium on a HisTrap (NiNTA) FF 5 mL prepacked column. The proteins were next eluted from the NiNTA resin by applying 500 mM imidazole and concentrated by centrifugation using NMWL filters (Nominal Molecular Weight Limit) with a cut-off of 10 kDa (FIGS. 15-16).

We conclude from these experiments that several of the Mk_{6P4}^{c7HopQ}V1-V4 Megakine variants (SEQ ID NO: 3-6) can be expressed as a well folded and functional chimeric protein and purified by conventional purification methods.

### Example 4

### Bacterial Expression and Purification of 50 kDa Fusion Proteins Built from a c7HopQ Scaffold Inserted into the β-strand β2-β3-connecting β-turn of a 6P4-CCL5 chemokine

As we were able to display a functional Megakine on the surface of yeast and express them as soluble proteins in yeast, we set out to express this 50 kDa fusion proteins in the periplasm of E. coli, purified them to homogeneity and determined their properties. In order to express Megakines Mk_{6P4-CCL5}^{c7HopQ}V1-V4 Megakine variant proteins (SEQ ID NO: 3-6) in the periplasm of E. coli, we used standard methods to construct a vector that allowed the expression of 6P4-CCL5 mega kines: scaffolds can be inserted into the β-turn connecting β-strand 2 (β2) and β-strand 3 (β3) of the 6P4-CCL5 chemokine. This vector is a derivative of pMESy4 (Pardon, 2014) and contains an open reading frame that encodes the following polypeptides: the DsbA leader sequence that directs the secretion of the Megakine to the periplasm of E. coli, the N-terminus until β-strand β2 of the 6P4-CCL5 chemokine, a multiple cloning site in which for this example the circularly permutated variant of HopQ (c7HopQ) was cloned, the C-terminus from β-strand β3 of the 6P4-CCL5 chemokine, the 6× His tag and the EPEA tag followed by the Amber stop codon. Any other suitable scaffold can be cloned in the multicloning site of this vector.

In order to express Megakines in the periplasm of E. coli and purify this recombinant protein to homogeneity, we used standard methods to construct vectors where DsbA leader sequence directs the expression of four His-tagged and EPEA-tagged Mk_{6P4-CCL5}^{c7HopQ}V1-V4 Megakine variants (SEQ ID NO:16-19) in the periplasm of E. coli under the transcriptional control of the pLac promotor. WK6 bacterial cells (WK6 is a su⁻ nonsuppressor strain) were grown in 3 liters 2×TY medium at 37° C. and induced by IPTG when cells reached log-growing phase. Periplasmic expression of the His-tagged and EPEA-tagged Mk_{6P4-CCL5}^{c7HopQ}V1-V4 Megakine variants (SEQ ID NO: 16-19) was continued overnight at 28° C. Cells were harvested by centrifugation and the recombinant Megakines were released from the periplasm using an osmotic shock (Pardon et al., 2014). Recombinant Megakines were then separated from the protoplasts by centrifugation and recovered from the clarified supernatant on a HisTrap FF 5 mL prepacked column. The protein was next eluted from the NiNTA resin by applying 500 mM imidazole and concentrated by centrifugation using NMWL filters (Nominal Molecular Weight Limit) with a cut-off of 10 kDa (FIG. 17). Expressed and purified to homogeneity MegaBody Mb_{Nb207}^{c7HopQ} (SEQ ID NO: 20) was used as a control for functional experiments.

We conclude from these experiments that some of the Mk_{6P4-CCL5}^{c7HopQ}V1-V4 Megakine variants (SEQ ID NO: 3-6) can be expressed as a well folded and functional chimeric protein in E. coli and purified by conventional purification methods.

### Example 5

### Cell-Based Assays Confirming the Functionality of 50 kDa fusion Proteins Built from a c7HopQ Scaffold Inserted into the β-strand β2-β3-connecting β-turn (40s Loop) of a 6P4-CCL5 chemokine

The conservation of functionality/proper folding of 6P4-CCL5 when presented in the c7HopQ scaffold was assessed by the ability of Mk_{6P4-CCL5}^{c7HopQ}V1-V4 Megakine variants to activate CCR5, the cognate receptor of CCL5. The activity was evaluated in cell-based assays monitoring the recruitment of β-arrestin-1 or miniGi (an engineered GTPase domain of Ga subunit; Wan et al., 2018) to CCR5 following agonist stimulation, based on the complementation of the split NanoLuciferase (NanoBiT-Promega) (Dixon A S et al. 2016 ACS Chem Biol.).

5×10⁶ HEK293T cells were plated in 10 cm-culture dishes and 24 hours later co-transfected with pNBe2 and pNBe3 vectors (Promega) encoding human CCR5 C-terminally fused to SmBiT (VTGYRLFEEIL) (Nanoluciferase subunit I) separated by a 15 Gly/Ser linker (GSSGGGGSGGGGSSG) and human β-arrestin-1 or miniGi N-terminally fused to LgBiT (Nanoluciferase subunit II, residues 1-156) followed by a 15 Gly/Ser linker, respectively. 24 hours post-transfection cells were harvested, incubated 25 minutes at 37° C. with 100-fold diluted Nano-Glo Live Cell substrate and distributed into white 96-well plates at 5×10⁴ cells/well. β-arrestin-1 or miniGi recruitment to CCR5 upon Megakine addition was evaluated via NanoLuciferase complementation and thus catalytic activity recovery measured with a Mithras LB940 luminometer (Berthold Technologies). The activity of non-purified periplasmic extracts and purified Mk_{6P4-CCL5}^{c7HopQ}V1-V4 Megakine variants selected from yeast display (SEQ ID NO: 16-19) was compared to the activity of the non-purified recombinant soluble 6P4-CCL5 chemokine (SEQ ID NO: 33) produced in mammalian cells (HEK293T) under the dependence of a CMV promoter using pIRES-puromycin vector.

6P4-CCL5 chemokine retains its functionality upon the insertion of the c7HopQ scaffold into its β2-β3-connecting β-strand, as demonstrated by the ability of Mk_{6P4-CCL5}^{c7HopQ}V1-V4 Megakine variants to induce concentration-dependent β-arrestin-1 and miniGi recruitment to CCR5 (FIG. 18).

### Example 6

### Design and Generation of Other of 50 kDa Fusion Proteins Built from a c7HopQ Scaffold Inserted into the β-strand β2-β3-connecting β-turn of a 6P4-CCL5 chemokine by In Vivo Selection

As the capacity to fold, but also the stability and the rigidity of Megakines may rely on the composition and the length of the polypeptide linkages that connect the chemokine to the scaffold, we introduced in vitro evolution techniques for the fine-tuning of particular Megakines formats if required. Starting from the Megakines described in Example 1, we constructed libraries encoding Megakines with a similar design in which two short peptides of variable length and mixed amino acid composition connect chemokine to scaffold according to FIG. 2 that are amenable to in vivo selection.

The 50 kDa Megakine described here is a chimeric polypeptide concatenated from parts of chemokine and parts of a scaffold protein connected according to FIGS. 2 and 3. Here, the chemokine used is the 6P4-CCL5, an agonist of CCR5 GPCR as depicted in SEQ ID NO: 1 (6P4-CCL5 is an analogue of the antagonist CCL55P7; Zheng et al. 2017; PDB code CCL55P7: 5UIW). The β-turn connecting β-strand 2 and β-strand 3 of 6P4-CCL5 was interrupted for fusion to the scaffold protein. The scaffold protein is an adhesin domain of Helicobacter pylori strain G27 (PDB: 5LP2; SEQ ID NO: 2) called HopQ (Javaheri et al, 2016). The N- and C-terminus of HopQ was connected, although after a truncation of seven amino acids in the circular permutation region (called c7HopQ) which otherwise appeared as a loop never fully visible in electron density of crystal structures. This truncated fusion creates a circularly permutated variant of HopQ, called c7HopQ, wherein a cleavage within the amino acid sequence was made somewhere else in its sequence (i.e. in a position corresponding to an accessible site in an exposed region of said scaffold protein). All parts were connected to each other from the amino to the carboxy terminus in the next given order by peptide bonds: N-terminus until β-strand 2 of the 6P4-CCL5 chemokine (1-44 of SEQ ID NO:1), a peptide linker of one or two amino acids with random composition, a C-terminal part of HopQ (residues 195-411 of SEQ ID NO:2), an N-terminal part of HopQ (residues 18-183 of SEQ ID NO:2), a peptide linker of one or two amino acids with random composition, the C-terminal part from β-strand 3 till end of the 6P4-CCL5 chemokine (47-69 of SEQ ID NO:1).

To display and select functional variants of the Megakines described in Examples 1 to 5 that differ in composition and length of the linkers connecting chemokine to scaffold on yeast, we used standard methods to construct a library of open reading frame that encode the various Megakines in fusion to a number of accessory peptides and proteins (SEQ ID NO:25-28) according to FIG. 7: the appS4 leader sequence that directs extracellular secretion in yeast (Rakestraw, 2009), N-terminus until β-strand 2 of the 6P4-CCL5 chemokine (1-44 of SEQ ID NO:1), a peptide linker of one or two amino acids with random composition, a C-terminal part of HopQ (residues 195-411 of SEQ ID NO:2), an N-terminal part of HopQ (residues 18-183 of SEQ ID NO:2), a peptide linker of one or two amino acids with random composition, the C-terminal part from β-strand 3 till end of the 6P4-CCL5 chemokine (47-69 of SEQ ID NO:1), a flexible (GGSG)ₙ peptide linker, the Aga2p the adhesion subunit of the yeast agglutinin protein Aga2p which attaches to the yeast cell wall through disulfide bonds to the Aga1p protein, an acyl carrier protein for the orthogonal fluorescent staining of the displayed fusion protein (Johnsson, 2005) followed by the cMyc Tag. This open reading frame was put under the transcriptional control of galactose-inducible GAL1/10 promotor into the pCTCON2 vector (Chao, 2006) to construct a yeast display library encoding 176400 different variants of the Megakines (See FIG. 7).

For in vitro selection, this library was introduced into yeast strain EBY100. Transformed cells were grown and induced overnight in a galactose-rich medium. Induced cells were orthogonally stained with coA-547 (2 μM) using the SFP synthase (1 μM) and incubated with 0.25 μg/mL Alexa Fluor® 647 fluorescently labelled anti-CCL5 monoclonal antibody (anti-CCL5-mAb647). Next, these cells were washed and subjected to 2-parameter FACS analysis to identify yeast cells that display high levels of a Megakine expression (high CoA-547 fluorescence) and bind the anti-CCL5-mAb647 (high Alexa Fluor® 647 fluorescence). Cells that display high levels of anti-CCL5-mAb647 binding were sorted and amplified in a glucose-rich medium to be subjected to following rounds of selection by yeast display and two-parameter FACS analysis (FIG. 8).

After two rounds of selection, a representative number of highly fluorescent cells in the CoA-547 and Alexa Fluor® 647 channels were grown as single colonies and subjected to DNA sequencing to determine the sequences of a representative number of peptide linkers connecting chemokine to scaffold protein. Two representative clones of each type of linkers with 1-1, 1-2, 2-1 and 2-2 amino acid short linker variants are presented in Table 1.

TABLE 1
Composition and length of some yeast-display optimized
linker peptides connecting scaffold protein c7HopQ to a chemokine.

| Megakine clone | Connection 1 | Connection 2 |
|---|---|---|
| MP1498_D12 | K | P |
| MP1498_A2 | G | S |
| MP1498_F5 | R | GP |
| MP1498_H11 | R | KA |
| MP1498_133 | KT | R |
| MP1498_G8 | EA | Q |
| MP1498_A5 | RG | KD |
| MP1498_A12 | YR | QP |

This demonstrates that different short peptide connections between chemokine and scaffold protein can be selected from Megakine libraries by in vivo selections using yeast-display and displayed as functional chemokine chimeric proteins on the surface of the yeast cell.

### Example 7

### Bacterial Expression and Purification of 50 kDa Fusion Proteins Built from a c7HopQ Scaffold Inserted into the β-strand β2-β3-connecting β-turn of a CXCL12 chemokine

As a second proof of concept of obtaining rigid fusion proteins ‘Megakines’, the CXCL12 chemokine, belonging to the subfamily of CXC-chemokines, was grafted onto a large scaffold protein via two peptide bonds that connect CXCL12 to a scaffold according to FIG. 2 to build a rigid Megakine.

The 50 kDa Megakine described here is a chimeric polypeptide concatenated from parts of chemokine and parts of a scaffold protein connected according to FIGS. 2 and 3. Here, the chemokine used is the CXCL12, also called SDF-1 which binds to and activates the CXCR4 GPCR as well as the ACKR3 GPCR, as depicted in SEQ ID NO: 22 (PDB code: 3HP3). The scaffold protein was inserted in the β-turn connecting β-strand 2 and β-strand 3 of CXCL12. The scaffold protein is an adhesin domain of Helicobacter pylori strain G27 (PDB 5LP2; SEQ ID NO: 2) called HopQ (Javaheri et al, 2016). The N- and C-terminus of HopQ was connected, although after a truncation of seven amino acids in the circular permutation region (called c7HopQ) which otherwise appeared as a loop never fully visible in electron density of crystal structures. This truncated fusion creates a circularly permutated variant of HopQ, called c7HopQ, wherein a cleavage within the amino acid sequence was made somewhere else in its sequence (i.e. in a position corresponding to an accessible site in an exposed region of said scaffold protein). In analogy with example 1, a low free energy Mk_{CXCL12}^{c7HopQ} (SEQ ID NO: 23) was generated, where all parts were connected as follows: the N-terminus until β-strand 2 of the CXCL12 chemokine (1-43 of SEQ ID NO:22), a C-terminal part of HopQ (residues 192-411 of SEQ ID NO: 2), an N-terminal part of HopQ (residues 18-184 of SEQ ID NO:2), the C-terminal part from β-strand 3 till end of the CXCL12 chemokine (45-68 of SEQ ID NO: 22), 6× His tag and EPEA tag (U.S. Pat. No. 9,518,084 B2).

We set out to express this 50 kDa fusion protein in the periplasm of E. coli, purified it to homogeneity and determined its properties. In order to express Megakine Mk_{CXCL12}^{c7HopQ} in the periplasm of E. coli, we used standard methods to construct a vector that allowed the expression of CXCL12 Megakines: scaffolds can be inserted into the β-turn connecting β-strand 2 (β2) and β-strand 3 (β3) of the CXCL12 chemokine. This vector is a derivative of pMESy4 (Pardon, 2014) and contains an open reading frame that encodes the following polypeptides: the DsbA leader sequence that directs the secretion of the Megakine to the periplasm of E. coli, the N-terminus until β-strand β2 of the CXCL12 chemokine, a multiple cloning site in which for this example the circularly permutated variant of HopQ (c7HopQ) was cloned, the C-terminus from β-strand β3 of the CXCL12 chemokine, the 6× His tag and the EPEA tag followed by the Amber stop codon (SEQ ID NO: 24). Any other suitable scaffold can be cloned in the multicloning site of this vector. Mk_{CXCL12}^{c7HopQ} expression is as described in example 4.

### Example 8

### Design and Generation of 94 kDa Fusion Protein Built from a YgjK Scaffold Inserted into the β-strand β2-β3-connecting β-turn of a 6P4-CCL5 chemokine

Building on the successful design of our first Megakines from a 6P4-CCL5 chemokine grafted onto c7HopQ (Examples 1 to 6), we also aimed at developing other Megakines designs built from chemokines that are connected to larger scaffold proteins.

The 94 kDa Megakine described here is a chimeric polypeptide concatenated from parts of chemokine and parts of a scaffold protein connected according to FIG. 2. Here, the chemokine used is the 6P4-CCL5, as used in previous examples, and as depicted in SEQ ID NO: 1. The β-turn connecting β-strand 2 and β-strand 3 of 6P4-CCL5 was interrupted for fusion to the scaffold protein. The scaffold protein is a 86 kDA periplasmic protein of E. coli (PDB code 3W7S, SEQ ID NO: 34) called YgjK (Kurakava et al, 2008). In the tertiary structure of YgjK, two antiparallel β-strands with surface accessible β-turns were identified: β-turn A′S1-A′52 and β-turn NS6-NS7. In order to generate distinct Megakines of 94 kDa MW, wherein the topology is (differently) interrupted, these two β-turns were truncated and an additional circular permutation was introduced to generate two scaffold proteins:

c1YgjK (SEQ ID NO: 36): the C-terminal part of YgjK (residues 464-760 of SEQ ID NO: 34), a short peptide linker (SEQ ID NO: 35) connecting the C-terminus and the N-terminus of YgjK to produce a circular permutant of the scaffold protein, the N-terminal part of YgjK (residues 1-461 of SEQ ID NO: 34)

c2YgjK (SEQ ID NO: 37): the C-terminal part of YgjK (residues 105-760 of SEQ ID NO: 34), a short peptide linker (SEQ ID NO: 35) connecting the C-terminus and the N-terminus of YgjK to produce a circular permutant of the scaffold protein, the N-terminal part of YgjK (residues 1-102 of SEQ ID NO: 34)

To design functional Megakine fusion protein variants, in silico molecular modelling using accessible crystal structures (PDB code CCL5-5P7: 5UIW, PDB code YgjK: 3W7S) was performed. As a result, three MegaKine Mk_{6P4-CCL5}^{c1YgjK} and two Mk_{6P4-CCL5}^{c2YgjK} models were generated, where all parts were connected to each other from the amino (N-) to the carboxy (C-) terminus in the next given order by peptide bonds:

Mk_{6P4-CCL5}^{c1YgjK}V1 (SEQ ID NO: 38, FIG. 20): N-terminus until β-strand 2 of the 6P4-CCL5 chemokine (1-45 of SEQ ID NO: 1), Gly-Gly two amino acid linker, c1YgjK scaffold protein (SEQ ID NO:36), Gly-Gly two amino acid linker, the C-terminal part from β-strand 3 till end of the 6P4-CCL5 chemokine (47-69 of SEQ ID NO: 1)

Mk_{6P4-CCL5}^{c1YgjK}V2 (SEQ ID NO:39, FIG. 21): N-terminus until β-strand 2 of the 6P4-CCL5 chemokine (1-45 of SEQ ID NO:1), Gly one amino acid linker, c1YgjK scaffold protein (SEQ ID NO:36), Gly one amino acid linker, the C-terminal part from β-strand 3 till end of the 6P4-CCL5 chemokine (47-69 of SEQ ID NO: 1)

Mk_{6P4-CCL5}^{c1YgjK}V3 (SEQ ID NO:40, FIG. 22): N-terminus until β-strand 2 of the 6P4-CCL5 chemokine (1-45 of SEQ ID NO:1), c1YgjK scaffold protein (SEQ ID NO:36), the C-terminal part from β-strand 3 till end of the 6P4-CCL5 chemokine (47-69 of SEQ ID NO: 1)

Mk_{6P4-CCL5}^{c2YgjK}V1 (SEQ ID NO:41, FIG. 23): N-terminus until β-strand 2 of the 6P4-CCL5 chemokine (1-45 of SEQ ID NO:1), Gly-Gly two amino acid linker, c2YgjK scaffold protein (SEQ ID NO:37), Gly-Gly two amino acid linker, the C-terminal part from β-strand 3 till end of the 6P4-CCL5 chemokine (47-69 of SEQ ID NO: 1)

Mk_{6P4-CCL5}^{2YgjK}V3 (SEQ ID NO:42, FIG. 24): N-terminus until β-strand 2 of the 6P4-CCL5 chemokine (1-45 of SEQ ID NO:1), c2YgjK scaffold protein (SEQ ID NO: 37), the C-terminal part from β-strand 3 till end of the 6P4-CCL5 chemokine (47-69 of SEQ ID NO: 1)

### Example 9

### Yeast Display of 94 kDa Fusion Proteins Built from c1YgjK and c2Ygjk Scaffolds Inserted into the β-Strand β2-β3-Connecting β-Turn of a 6P4-CCL5 Chemokine

To demonstrate that these five Mk_{6P4-CCL5}^{c1YgjK}V1-V3 and Mk_{6P4-CCL5}^{c2YgjK}V1/V3 Megakine variants (SEQ ID NO:38-42) can be expressed as correctly-folded and functional proteins, we displayed these proteins on the surface of yeast (Boder, 1997) as performed for Mk_{6P4-CCL5}^{c7HopQ}V1-V4 Megakine variants (Example 2). Proper folding of 6P4-CCL5 chemokine part was examined by using a fluorescent conjugated monoclonal antibody that binds to functional 6P4-CCL5 chemokine (Alexa Fluor® 647 anti-human RANTES (CCL5) Antibody from Biolegend, ref 515506; anti-CCL5-mAb647). In order to display the Mk_{6P4-CCL5}^{c1YgjK}V1-V3 and Mk_{6P4-CCL5}^{2YgjK}V1/V3 Megakine variants on yeast, we used standard methods to construct an open reading frame that encodes the Megakine in fusion to a number of accessory peptides and proteins for yeast display (SEQ ID NO:43-47): the appS4 leader sequence that directs extracellular secretion in yeast (Rakestraw, 2009), Mk_{6P4-CCL5}^{c1YgjK} or Mk_{6P4-CCL5}^{c2YgjK} Megakine variant, a flexible peptide linker, the Aga2p the adhesion subunit of the yeast agglutinin protein Aga2p which attaches to the yeast cell wall through disulfide bonds to the Aga1p protein, an acyl carrier protein for the orthogonal fluorescent staining of the displayed fusion protein (Johnsson, 2005) followed by the cMyc Tag. This open reading frame was put under the transcriptional control of galactose-inducible GAL1/10 promotor into the pCTCON2 vector (Chao, 2006) and introduced into yeast strain EBY100.

EBY100 yeast cells, bearing this plasmid, were grown and induced overnight in a galactose-rich medium to trigger the expression and secretion of the Mk_{6P4-CCL5}^{c1/2YgjK}-Aga2p-ACP fusion. For the orthogonal staining of ACP, cells were incubated for 1 h in the presence a fluorescently labelled CoA analogue (CoA-547, 2 μM) and catalytic amounts of the SFP synthase (1 μM). To analyse the functionality of the displayed Megakine, we examined its ability to be recognized by Alexa Fluor® 647 fluorescently labelled anti-CCL5 monoclonal antibody (anti-CCL5-mAb647) by flow cytometry. Accordingly, EBY100 yeast cells were induced and fluorescently stained orthogonally with CoA547 to monitor the display of Mk_{6P4-CCL5}^{c1/2YgjK}-Aga2p-ACP fusions. Yeast cells that display Mk_{6P4-CCL5}^{c7HopQ}V4 (SEQ ID NO: 10, Example 2) were used as an additional positive control. These orthogonally stained yeast cells were next incubated 1 h in the presence of anti-CCL5-mAb647 (at concentration of 80 ng/mL). In these experiments, induced yeast cells were washed and subjected to flow-cytometry to measure the Megakine display level of each cell by comparing the CoA547-fluorescence level to yeast cells that display the Mega body Mb_{Nb207}^{cHopQ}-Aga2p-ACP fusion (SEQ ID NO:11; wherein a Megabody is similar to a Megakine, but instead of a chemokine a Nanobody (Nb) is fused to a scaffold protein, with herein Nb₂₀₇ as a GFP-specific Nb) and were stained orthogonally in the same way. Indeed, for all 5 Mk_{6P4-CCL5}^{c1/2YgjK} variants, the quantified display levels of Mk_{6P4-CCL5}^{c1/2YgjK}-Aga2p-ACP fusions were approximately 70% (FIG. 25A-FIG. 25H).

Next, the binding of anti-CCL5-mAb647 was analyzed by examination of 647-fluorescence level that should be linearly correlated to the expression level of Mk_{6P4-CCL5}^{c1/2YgjK} variants on the surface of yeast. A two-dimensional flow cytometric analysis confirmed that anti-CCL5-mAb647 (high 647-fluorescence level) only binds to yeast cells with significant Megakine display levels (high CoA547-fluorescence level), with the greatest linear fit for Mk_{6P4-CCL5}^{c2YgjK}V1 (SEQ ID NO: 46) and Mk_{6P4-CCL5}^{c2YgjK}V3 (SEQ ID NO:47) probably due to best accessibility of the epitope recognized by the anti-CCL5-mAb647 (FIG. 26A-FIG. 26H). In contrast, anti-CCL5-mAb647 does not bind to yeast cells that display Megabody Mb_{Nb207}^{cHopQ}-Aga2p-ACP fusion (SEQ ID NO: 11; GFP-specific Megabody as negative control) and have been stained in the same way.

We conclude from these experiments that all five Mk_{6P4-CCL5}^{c1YgjK}V1-V3 and Mk_{6P4-CCL5}^{c2YgjK}V1/V3 Megakine variants (SEQ ID NO: 38-42), possessing two different fusion scaffolds can be expressed as a well-folded and functional chimeric protein on the surface of yeast.

### Example 10

### Design and Generation of 58 kDa Fusion Protein Built from a HopQ Scaffold Inserted into the β-strand β6-β7-connecting β-turn of an IL-1β interleukin

Building on the successful design of our first Megakines from a 6P4-CCL5 and CXCL12 chemokine grafted onto c7HopQ (Examples 1 to 7) and c1YgjK/c2Ygjk (Examples 8 and 9) scaffolds, we also aimed at developing other Megakines designs built from another class of cytokines, interleukins in particular, that are connected to larger scaffolds.

The 58 kDa Megakine described here is a chimeric polypeptide concatenated from parts of interleukin and parts of a scaffold protein connected according to FIG. 27. Here, the interleukin used is the human IL-1β (SEQ NO: 48), belonging to the subfamily of interleukins that exerts its effects through IL-1 receptor type I (IL-1RI) and IL-1 receptor accessory protein (IL-1RAcP) (PDB 3O4O, Wang et al, 2010). In the functional IL-1β•IL-1RI•IL-1RAcP complex, the β-turn connecting β-strand β6 and β-strand β7 of IL-1β is exposed to the solvent and therefore, accessible for the scaffold protein fusion (FIG. 28). The scaffold protein is c7HopQ scaffold used to generate 6P4-CCL5 chemokine-based Megakines (Examples 1 to 6).

To design functional MkIL-1βc7HopQ Megakine fusion protein variants, in silico molecular modelling using accessible crystal structures (PDB code IL-1β: 3O4O, PDB code HopQ: 5LP2) was performed. As a result, three Mk_{IL-1β}^{c7HopQ} models were generated, where all parts were connected to each other from the amino (N-) to the carboxy (C-) terminus in the next given order by peptide bonds:

Mk_{IL-1β}^{c7HopQ}V1 (SEQ ID NO: 49, FIG. 29): N-terminus until β-strand β6 of the human IL-1β interleukin (1-73 of SEQ ID NO: 48), Gly-Gly two amino acid linker, a C-terminal part of HopQ (residues 193-411 of SEQ ID NO:2), an N-terminal part of HopQ (residues 18-185 of SEQ ID NO: 2), Gly-Gly two amino acid linker, the C-terminal part from β-strand β7 of the human IL-1β interleukin (78-153 of SEQ ID NO:48)

Mk_{IL-1β}^{c7HopQ}V2 (SEQ ID NO:50, FIG. 30): N-terminus until β-strand β6 of the human IL-1β interleukin (1-73 of SEQ ID NO:48), Gly one amino acid linker, a C-terminal part of HopQ (residues 193-411 of SEQ ID NO: 2), an N-terminal part of HopQ (residues 18-185 of SEQ ID NO: 2), Gly one amino acid linker, the C-terminal part from β-strand β7 of the human IL-1β interleukin (78-153 of SEQ ID NO: 48)

Mk_{IL-1β}^{c7HopQ}V3 (SEQ ID NO: 51, FIG. 31): N-terminus until β-strand β6 of the human IL-1β interleukin (1-73 of SEQ ID NO: 48), a C-terminal part of HopQ (residues 193-411 of SEQ ID NO: 2), an N-terminal part of HopQ (residues 18-185 of SEQ ID NO: 2), the C-terminal part from β-strand β7 of the human IL-1β interleukin (78-153 of SEQ ID NO: 48)

### Example 11

### Yeast Display of 58 kDa Fusion Proteins Built from a HopQ Scaffold Inserted into the β-strand β6-β7-connecting β-turn of a IL-1β interleukin

To demonstrate that three Mk_{IL-1β}^{c7HopQ} Megakine variants (SEQ ID NO: 49-51) can be expressed as correctly folded and functional proteins, yeast surface display of these proteins (Boder, 1997) as performed for Mk_{6P4-CCL5}^{c7HopQ} Megakine variants (Example 2) and Mk_{6P4-CCL5}^{cYgjkA/B} Megakine variants (Example 9) is required. The proper folding of IL-1β interleukin part can be examined using a fluorescent conjugated monoclonal antibody that binds to functional IL-1β interleukin (Alexa Fluor® 647 anti-human IL-1β Antibody (CRM46) from Life Technologies, ref 51-7018-42). In order to display the Mk_{IL-1β}^{c7HopQ}V1-V3 Megakine variants on yeast, standard methods to construct an open reading frame that encodes the Megakine in fusion to a number of accessory peptides and proteins (SEQ ID NO:52-54) are used: the appS4 leader sequence that directs extracellular secretion in yeast (Rakestraw, 2009), Mk_{IL-1β}^{c7HopQ} Megakine variant, a flexible peptide linker, the Aga2p the adhesion subunit of the yeast agglutinin protein Aga2p which attaches to the yeast cell wall through disulfide bonds to the Aga1p protein, an acyl carrier protein for the orthogonal fluorescent staining of the displayed fusion protein (Johnsson, 2005) followed by the cMyc Tag. This open reading frame under the transcriptional control of galactose-inducible GAL1/10 promotor is then cloned into the pCTCON2 vector (Chao, 2006) and introduced into yeast strain EBY100. EBY100 yeast cells, bearing this plasmid, are grown and induced overnight in a galactose-rich medium to trigger the expression and secretion of the Mk_{IL-1β}^{c7HopQ}-Aga2p-ACP fusion. For the orthogonal staining of ACP, as shown in previous examples, cells are incubated for 1 h in the presence a fluorescently labelled CoA analogue (CoA-547, 2 μM) and catalytic amounts of the SFP synthase (1 μM). To analyze the functionality of the displayed Megakine, its ability to be recognized by Alexa Fluor® 647 fluorescently labelled IL-1β monoclonal antibody (anti-human IL-1β antibody CRM46) is monitored by flow cytometry. Accordingly, EBY100 yeast cells are induced and fluorescently stained orthogonally with CoA547 to monitor the display of Mk_{IL-1β}^{c7HopQ}-Aga2p-ACP fusions. Yeast cells that display IL-1β interleukin (SEQ ID NO: 55) form an additional positive control. These orthogonally stained yeast cells are then next incubated 1 h in the presence of anti-human IL-1β antibody CRM46 (at concentration of 80 ng/mL). In these experiments, induced yeast cells are washed and subjected to flow-cytometry to measure the Megakine display level of each cell by comparing the CoA547-fluorescence level to yeast cells that display the Megabody Mb_{Nb207}^{cHopQ}-Aga2p-ACP fusion (SEQ ID NO: 11; wherein a Megabody is similar to a Megakine, but instead of a interleukin a Nanobody (Nb) is fused to a scaffold protein, with herein Nb₂₀₇ as a GFP-specific Nb) and are stained orthogonally in the same way. Next, the binding of anti-human IL-1β antibody CRM46 can be analyzed by examination of 647-fluorescence level that should be linearly correlated to the expression level of Mk_{IL-1β}^{c7HopQ} variants on the surface of yeast. A two-dimensional flow cytometric analysis confirmed that anti-human IL-1β antibody CRM46 (high 647-fluorescence level) only binds to yeast cells with significant Megakine display levels (high CoA547-fluorescence level). In contrast, anti-human IL-1β antibody CRM46 does not bind to yeast cells that display Megabody Mb_{Nb207}^{cHopQ}-Aga2p-ACP fusion (SEQ ID NO:11) and have been stained in the same way.

Sequence listing
>SEQ ID NO: 1: 6P4-CCL5 chemokine
>SEQ ID NO: 2: Helicobacter pylori strain G27 HopQ adhesin domain protein (PDB 5LP2)
>SEQ ID NO: 3: Mk_{6P4-CCL5}^{c7HopQ}V1 Megakine
(N-terminus of 6P4-CCL5-chemokine, HopQ sequences underlined, C-terminus of 6P4-CCL5 chemokine
in bold, 6xHis tag, EPEA tag)
QGPPGDIVLACCFAYIARPLPRAHIKEYFYTSGKCSNPAVVFVKTTTSVIDTTNDAQNLLTQAQTIVNTLKDYCPILIAKSS
SSNGGTNNANTPSWQTAGGGKNSCATFGAEFSAASDMINNAQKIVQETQQLSANQPKNITQPHNLNLNSPSSLTAL
AQKMLKNAQSQAEILKLANQVESDFNKLSSGHLKDYIGKCDASAISSANMTMQNQKNNWGNGCAGVEETQSLLKT
SAADFNNQTPQINQAQNLANTLIQELGNNTYEQLSRLLTNDNGTNSKTSAQAINQAVNNLNERAKTLAGGTTNSPA
YQATLLALRSVLGLWNSMGYAVICGGYTKSPGENNQKDFHYTDENGNGTTINCGGSTNSNGTHSYNGTNTLKADKN
VSLSIEQYEKIHEAYQILSKALKQAGLAPLNSKGEKLEAHVTTSKNRQVCANPEKKWVREYINSLEMShhhhhhepea
>SEQ ID NO: 4: Mk_{6P4-CCL5}^{c7HopQ}V2 Megakine
(N-terminus of 6P4-CCL5-chemokine, _T short peptide linker, HopQ sequences underlined, C-terminus of
6P4-CCL5 chemokine in bold, 6xHis tag, EPEA tag)
QGPPGDIVLACCFAYIARPLPRAHIKEYFYTSGKCSNPAVVFVTTTTTSVIDTTNDAQNLLTQAQTIVNTLKDYCPILIAKS
SSSNGGTNNANTPSWQTAGGGKNSCATFGAEFSAASDMINNAQKIVQETQQLSANQPKNITQPHNLNLNSPSSLTA
LAQKMLKNAQSQAEILKLANQVESDFNKLSSGHLKDYIGKCDASAISSANMTMQNQKNNWGNGCAGVEETQSLLK
TSAADFNNQTPQINQAQNLANTLIQELGNNTYEQLSRLLTNDNGTNSKTSAQAINQAVNNLNERAKTLAGGTTNSP
AYQATLLALRSVLGLWNSMGYAVICGGYTKSPGENNQKDFHYTDENGNGTTINCGGSTNSNGTHSYNGTNTLKADK
NVSLSIEQYEKIHEAYQILSKALKQAGLAPLNSKGEKLEAHVTTSKNRQVCANPEKKWVREYINSLEMShhhhhhepea
>SEQ ID NO: 5: Mk_{6P4-CCL5}^{c7HopQ}V3 Megakine
(N-terminus of 6P4-CCL5-chemokine, HopQ sequences underlined, C-terminus of 6P4-CCL5 chemokine
in bold, 6xHis tag, EPEA tag)
QGPPGDIVLACCFAYIARPLPRAHIKEYFYTSGKCSNPAVVFVTRTKTTSVIDTTNDAQNLLTQAQTIVNTLKDYCPILIA
KSSSSNGGTNNANTPSWQTAGGGKNSCATFGAEFSAASDMINNAQKIVQETQQLSANQPKNITQPHNLNLNSPSSL
TALAQKMLKNAQSQAEILKLANQVESDFNKLSSGHLKDYIGKCDASAISSANMTMQNQKNNWGNGCAGVEETQSL
LKTSAADFNNQTPQINQAQNLANTLIQELGNNTYEQLSRLLTNDNGTNSKTSAQAINQAVNNLNERAKTLAGGTTNS
PAYQATLLALRSVLGLWNSMGYAVICGGYTKSPGENNQKDFHYTDENGNGTTINCGGSTNSNGTHSYNGTNTLKAD
KNVSLSIEQYEKIHEAYQILSKALKQAGLAPLNSKGEKLEAHVTTSKNRQVCANPEKKWVREYINSLEMShhhhhhepe
a
>SEQ ID NO: 6: Mk_{6P4-CCL5}^{c7HopQ}V4 Megakine
(N-terminus of 6P4-CCL5 chemokine, HopQ sequences underlined, C-terminus of 6P4-CCL5 chemokine
in bold, 6xHis tag, EPEA tag)
QGPPGDIVLACCFAYIARPLPRAHIKEYFYTSGKCSNPAVVFVTKTTSVIDTTNDAQNLLTQAQTIVNTLKDYCPILIAKSS
SSNGGTNNANTPSWQTAGGGKNSCATFGAEFSAASDMINNAQKIVQETQQLSANQPKNITQPHNLNLNSPSSLTAL
AQKMLKNAQSQAEILKLANQVESDFNKLSSGHLKDYIGKCDASAISSANMTMQNQKNNWGNGCAGVEETQSLLKT
SAADFNNQTPQINQAQNLANTLIQELGNNTYEQLSRLLTNDNGTNSKTSAQAINQAVNNLNERAKTLAGGTTNSPA
YQATLLALRSVLGLWNSMGYAVICGGYTKSPGENNQKDFHYTDENGNGTTINCGGSTNSNGTHSYNGTNTLKADKN
VSLSIEQYEKIHEAYQILSKALKQAGLAPLNSKGEKLEAHVTTSKNRQVCANPEKKWVREYINSLEMShhhhhhepea
>SEQ ID NO: 7: Mk_{6P4-CCL5}^{c7HopQ}V1_Aga2p_ACP protein sequence
(appS4 leader sequence, Megakine Mk_{6P4-CCL5}^{c7HopQ}V1 depicted in bold, flexible (GGGS)ₙ polypeptide
linker, Aga2p protein sequence underlined, ACP sequence double underlined, cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAQGPPGDIVLACCFAYIARPLPRAHIKEYFYTSGKCSNPAVVFVKTTTSVIDTTNDAQNLLTQAQTIVNT
LKDYCPILIAKSSSSNGGTNNANTPSWQTAGGGKNSCATFGAEFSAASDMINNAQKIVQETQQLSANQPKNITQP
HNLNLNSPSSLTALAQKMLKNAQSQAEILKLANQVESDFNKLSSGHLKDYIGKCDASAISSANMTMQNQKNNWG
NGCAGVEETQSLLKTSAADFNNQTPQINQAQNLANTLIQELGNNTYEQLSRLLTNDNGTNSKTSAQAINQAVNNL
NERAKTLAGGTTNSPAYQATLLALRSVLGLWNSMGYAVICGGYTKSPGENNQKDFHYTDENGNGTTINCGGSTN
SNGTHSYNGTNTLKADKNVSLSIEQYEKIHEAYQILSKALKQAGLAPLNSKGEKLEAHVTTSKNRQVCANPEKKWV
REYINSLEMSIgggsggggsggggsggggsggggsggggsggggsQELTTICEQIPSPTLESTPYSLSTTTILANGKAMQGVFE
YYKSVTFVSNCGSHPSTTSKGSPINTQYVFKdnsstsMSTIEERVKKIIGEQLGVKQEEVTNNASFVEDLGADSLDTVELV
MALEEEFDTEIPDEEAEKITTVGAAIDYINGHQAseqkliseedI
>SEQ ID NO: 8: Mk_{6P4-CCL5}^{c7HopQ}V2_Aga2p_ACP protein sequence
>SEQ ID NO: 9: Mk_{6P4-CCL5}^{c7HopQ}V3_Aga2p_ACP protein sequence
>SEQ ID NO: 10: Mk_{6P4-CCL5}^{c7HopQ}V4_Aga2p_ACP protein sequence
>SEQ ID NO: 11: Mb_{Nb207}^{c7HopQ}_Aga2p_ACP protein sequence
(appS4 leader sequence, MegaBody Mb_{Nb207}^{c7Hop} depicted in bold, flexible (GGGS)ₙ polypeptide linker,
Aga2p protein sequence underlined, ACP sequence double underlined, cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAQVQLVESGGGLVQTKTTTSVIDTTNDAQNLLTQAQTIVNTLKDYCPILIAKSSSSNGGTNNANTPSW
QTAGGGKNSCATFGAEFSAASDMINNAQKIVQETQQLSANQPKNITQPHNINLNSPSSLTALAQKMLKNAQSQA
EILKLANQVESDFNKLSSGHLKDYIGKCDASAISSANMTMQNQKNNWGNGCAGVEETQSLLKTSAADFNNQTPQ
INQAQNLANTLIQELGNNPFRASGGGSGGGGSGKLSDTYEQLSRLLTNDNGTNSKTSAQAINQAVNNLNERAKTL
AGGTTNSPAYQATLLALRSVLGLWNSMGYAVICGGYTKSPGENNQKDFHYTDENGNGTTINCGGSTNSNGTHSY
NGTNTLKADKNVSLSIEQYEKIHEAYQILSKALKQAGLAPLNSKGEKLEAHVTTSKYGSLRLSCAASGRTFSTAAMG
WFRQAPGKERDFVAGIYWTVGSTYYADSAKGRFTISRDNAKNTVYLQMDSLKPEDTAVYYCAARRRGFTLAPTR
ANEYDYWGQGTQVTVSSIgggsggggsggggsggggsggggsggggsggggsQELTTICEQIPSPTLESTPYSLSTTTILANG
KAMQGVFEYYKSVTFVSNCGSHPSTTSKGSPINTQYVFKdnsstsMSTIEERVKKIIGEQLGVKQEEVTNNASFVEDLGA
DSLDTVELVMALEEEFDTEIPDEEAEKITTVQAAIDYINGHQAseqkliseedI
>SEQ ID NO: 12: Mk_{6P4-CCL5}^{c7HopQ}V1 yeast secreted protein sequence
(appS4 leader sequence, Megakine Mk_{6P4-CCL5}^{c7HopQ}V1 depicted in bold, 6xHis tag, EPEA tag)
MRFPSIFTAVVFAASSALAAPANTTAEDETAQIPAEAVIGYLGLEGDSDVAALPLSDSTNNGSLSTNTTIASIAAKEEGV
QLDKREAEAQGPPGDIVLACCFAYIARPLPRAHIKEYFYTSGKCSNPAVVFVKTTTSVIDTTNDAQNLLTQAQTIVNT
LKDYCPILIAKSSSSNGGTNNANTPSWQTAGGGKNSCATFGAEFSAASDMINNAQKIVQETQQLSANQPKNITQP
HNLNLNSPSSLTALAQKMLKNAQSQAEILKLANQVESDFNKLSSGHLKDYIGKCDASAISSANMTMQNQKNNWG
NGCAGVEETQSLLKTSAADFNNQTPQINQAQNLANTLIQELGNNTYEQLSRLLTNDNGTNSKTSAQAINQAVNNL
NERAKTLAGGTTNSPAYQATLLALRSVLGLWNSMGYAVICGGYTKSPGENNQKDFHYTDENGNGTTINCGGSTN
SNGTHSYNGTNTLKADKNVSLSIEQYEKIHEAYQILSKALKQAGLAPLNSKGEKLEAHVTTSKNRQVCANPEKKWV
REYINSLEMShhhhhhepea
>SEQ ID NO: 13: Mk_{6P4-CCL5}^{c7HopQ}V2 yeast secreted protein sequence
>SEQ ID NO: 14: Mk_{6P4-CCL5}^{c7HopQ}V3 yeast secreted protein sequence
>SEQ ID NO: 15: Mk_{6P4-CCL5}^{c7HopQ}V4 yeast secreted protein sequence
>SEQ ID NO: 16: DsbA_Mk_{6P4-CCL5}^{c7HopQ}V1 protein sequence
(DsbA leader sequence, Megakine Mk_{6P4-CCL5}^{c7HopQ}V1 depicted in bold, 6xHis tag, EPEA tag)
MKKIWLALAGLVLAFSASAQGPPGDIVLACCFAYIARPLPRAHIKEYFYTSGKCSNPAVVFVKTTTSVIDTTNDAQNL
LTQAQTIVNTLKDYCPILIAKSSSSNGGTNNANTPSWQTAGGGKNSCATFGAEFSAASDMINNAQKIVQETQQLS
ANQPKNITQPHNLNLNSPSSLTALAQKMLKNAQSQAEILKLANQVESDFNKLSSGHLKDYIGKCDASAISSANMT
MQNQKNNWGNGCAGVEETQSLLKTSAADFNNQTPQINQAQNLANTLIQELGNNTYEQLSRLLTNDNGTNSKTS
AQAINQAVNNLNERAKTLAGGTTNSPAYQATLLALRSVLGLWNSMGYAVICGGYTKSPGENNQKDFHYTDENG
NGTTINCGGSTNSNGTHSYNGTNTLKADKNVSLSIEQYEKIHEAYQILSKALKQAGLAPLNSKGEKLEAHVTTSKNR
QVCANPEKKWVREYINSLEMShhhhhhepea
>SEQ ID NO: 17: DsbA_Mk_{6P4-CCL5}^{c7HopQ}V2 protein sequence
>SEQ ID NO: 18: DsbA_Mk_{6P4-CCL5}^{c7HopQ}V3 protein sequence
>SEQ ID NO: 19: DsbA_Mk_{6P4-CCL5}^{c7HopQ}V4 protein sequence
>SEQ ID NO: 20: DsbA_Mb_{Nb207}^{c7HopQ} Mega Body
(DsbA leader sequence, MegaBody Mb_{Nb207}^{c7HopQ} depicted in bold, 6xHis tag, EPEA tag)
MKKIWLALAGLVLAFSASAQVQLVESGGGLVQTKTTTSVIDTTNDAQNLLTQAQTIVNTLKDYCPILIAKSSSSNGGT
NNANTPSWQTAGGGKNSCATFGAEFSAASDMINNAQKIVQETQQLSANQPKNITQPHNLNLNSPSSLTALAQK
MLKNAQSQAEILKLANQVESDFNKLSSGHLKDYIGKCDASAISSANMTMQNQKNNWGNGCAGVEETQSLLKTSA
ADFNNQTPQINQAQNLANTLIQELGNNTYEQLSRLLTNDNGTNSKTSAQAINQAVNNLNERAKTLAGGTTNSPAY
QATLLALRSVLGLWNSMGYAVICGGYTKSPGENNQKDFHYTDENGNGTTINCGGSTNSNGTHSYNGTNTLKADK
NVSLSIEQYEKIHEAYQILSKALKQAGLAPLNSKGEKLEAHVTTSKYGSLRLSCAASGRTFSTAAMGWFRQAPGKER
DFVAGIYWTVGSTYYADSAKGRFTISRDNAKNTVYLQMDSLKPEDTAVYYCAARRRGFTLAPTRANEYDYWGQG
TQVTVSShhhhhhepea
>SEQ ID NO: 21: affinity tag (U.S. Pat. No. 9,518,084 B2)
>SEQ ID NO: 22: CXCL12 chemokine (Human)
>SEQ ID NO: 23: Mk_{CXCL12}^{c7HopQ} protein sequence
(CXCL12 depicted in bold, c7HopQ in normal text, 6xHis tag, EPEA tag dotted underlined)
KPVSLSYRCPCRFFESHVARANVKHLKILNTPNCALQIVARLKTKTTTSVIDTTNDAQNLLTQAQTIVNTLKDYCPILIA
KSSSSNGGTNNANTPSWQTAGGGKNSCATFGAEFSAASDMINNAQKIVQETQQLSANQPKNITQPHNLNLNSPSSL
TALAQKMLKNAQSQAEILKLANQVESDFNKLSSGHLKDYIGKCDASAISSANMTMQNQKNNWGNGCAGVEETQSL
LKTSAADFNNQTPQINQAQNLANTLIQELGNNTYEQLSRLLTNDNGTNSKTSAQAINQAVNNLNERAKTLAGGTTNS
PAYQATLLALRSVLGLWNSMGYAVICGGYTKSPGENNQKDFHYTDENGNGTTINCGGSTNSNGTHSYNGTNTLKAD
KNVSLSIEQYEKIHEAYQILSKALKQAGLAPLNSKGEKLEAHVTTSKNRQVCIDPKLKWIQEYLEKALNK

[symbol]

[symbol]

>SEQ ID NO: 24: DsbA-Mk_{CXCL12}^{c7HopQ} protein sequence
(DsbA leader sequence underlined, Mk_{CXCL12}^{c7HopQ}: CXCL12 depicted in bold, c7HopQ in normal text; 6xHis
tag, EPEA tag dotted underlined)
MKKIWLALAGLVLAFSASAKPVSLSYRCPCRFFESHVARANVKHLKILNTPNCALQIVARLKTKTTTSVIDTTNDAQNL
LTQAQTIVNTLKDYCPILIAKSSSSNGGTNNANTPSWQTAGGGKNSCATFGAEFSAASDMINNAQKIVQETQQLSAN
QPKNITQPHNLNLNSPSSLTALAQKMLKNAQSQAEILKLANQVESDFNKLSSGHLKDYIGKCDASAISSANMTMQNQ
KNNWGNGCAGVEETQSLLKTSAADFNNQTPQINQAQNLANTLIQELGNNTYEQLSRLLTNDNGTNSKTSAQAINQA
VNNLNERAKTLAGGTTNSPAYQATLLALRSVLGLWNSMGYAVICGGYTKSPGENNQKDFHYTDENGNGTTINCGGS
TNSNGTHSYNGTNTLKADKNVSLSIEQYEKIHEAYQILSKALKQAGLAPLNSKGEKLEAHVTTSKNRQVCIDPKLKWIQ
EYLEKALNK [symbol]
>SEQ ID NO: 25: Mk_{6P4-CCL5}^{c7HopQ} random linkers
(appS4 leader sequence, Megakine Mk_{6P4-CCL5}^{c7HopQ} YD1 depicted in bold, X is a short peptide linker of 1
AA and random composition, flexible (GGGS)ₙ polypeptide linker, Aga2p protein sequence underlined,
ACP sequence double underlined, cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAQGPPGDIVLACCFAYIARPLPRAHIKEYFYTSGKCSNPAVVFVTXTTSVIDTTNDAQNLLTQAQTIVNT
LKDYCPILIAKSSSSNGGTNNANTPSWQTAGGGKNSCATFGAEFSAASDMINNAQKIVQETQQLSANQPKNITQP
HNLNLNSPSSLTALAQKMLKNAQSQAEILKLANQVESDFNKLSSGHLKDYIGKCDASAISSANMTMQNQKNNWG
NGCAGVEETQSLLKTSAADFNNQTPQINQAQNLANTLIQELGNNTYEQLSRLLTNDNGTNSKTSAQAINQAVNNL
NERAKTLAGGTTNSPAYQATLLALRSVLGLWNSMGYAVICGGYTKSPGENNQKDFHYTDENGNGTTINCGGSTN
SNGTHSYNGTNTLKADKNVSLSIEQYEKIHEAYQILSKALKQAGLAPLNSKGEKLEAHVTTXNRQVCANPEKKWVR
EYINSLEMSgsgggsggggsggggsggggsggggsggggsggggsQELTTICEQIPSPTLESTPYSLSTTTILANGKAMQGVFE
YYKSVTFVSNCGSHPSTTSKGSPINTQYVFKdnsstsMSTIEERVKKIIGEQLGVKQEEVTNNASFVEDLGADSLDTVELV
MALEEEFDTEIPDEEAEKITTVQAAIDYINGHQAseqkliseedI
>SEQ ID NO: 26: Mk_{6P4-CCL5}^{c7HopQ} random linkers
(appS4 leader sequence, Megakine Mk_{6P4-CCL5}^{c7HopQ} YD1 depicted in bold, X is a short peptide linker of 1
AA and random composition and XX is a short peptide linker of 2 AA and random composition, flexible
(GGGS)ₙ polypeptide linker, Aga2p protein sequence underlined, ACP sequence double underlined,
cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAQGPPGDIVLACCFAYIARPLPRAHIKEYFYTSGKCSNPAVVFVTXTTSVIDTTNDAQNLLTQAQTIVNT
LKDYCPILIAKSSSSNGGTNNANTPSWQTAGGGKNSCATFGAEFSAASDMINNAQKIVQETQQLSANQPKNITQP
HNLNLNSPSSLTALAQKMLKNAQSQAEILKLANQVESDFNKLSSGHLKDYIGKCDASAISSANMTMQNQKNNWG
NGCAGVEETQSLLKTSAADFNNQTPQINQAQNLANTLIQELGNNTYEQLSRLLTNDNGTNSKTSAQAINQAVNNL
NERAKTLAGGTTNSPAYQATLLALRSVLGLWNSMGYAVICGGYTKSPGENNQKDFHYTDENGNGTTINCGGSTN
SNGTHSYNGTNTLKADKNVSLSIEQYEKIHEAYQILSKALKQAGLAPLNSKGEKLEAHVTTXXNRQVCANPEKKWV
REYINSLEMSgsgggsggggsggggsggggsggggsggggsggggsQELTTICEQIPSPTLESTPYSLSTTTILANGKAMQGVF
EYYKSVTFVSNCGSHPSTTSKGSPINTQYVFKdnsstsMSTIEERVKKIIGEQLGVKQEEVTNNASFVEDLGADSLDTVEL
VMALEEEFDTEIPDEEAEKITTVQAAIDYINGHQAseqkliseedI
>SEQ ID NO: 27: Mk_{6P4-CCL5}^{c7HopQ} random linkers
(appS4 leader sequence, Megakine Mk_{6P4-CCL5}^{c7HopQ} YD1 depicted in bold, XX is a short peptide linker of
2 AA and random composition and X is a short peptide linker of 1 AA and random composition, flexible
(GGGS)ₙ polypeptide linker, Aga2p protein sequence underlined, ACP sequence double underlined,
cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAQGPPGDIVLACCFAYIARPLPRAHIKEYFYTSGKCSNPAVVFVTXXTTSVIDTTNDAQNLLTQAQTIVN
TLKDYCPILIAKSSSSNGGTNNANTPSWQTAGGGKNSCATFGAEFSAASDMINNAQKIVQETQQLSANQPKNITQ
PHNLNLNSPSSLTALAQKMLKNAQSQAEILKLANQVESDFNKLSSGHLKDYIGKCDASAISSANMTMQNQKNNW
GNGCAGVEETQSLLKTSAADFNNQTPQINQAQNLANTLIQELGNNTYEQLSRLLTNDNGTNSKTSAQAINQAVN
NLNERAKTLAGGTTNSPAYQATLLALRSVLGLWNSMGYAVICGGYTKSPGENNQKDFHYTDENGNGTTINCGGST
NSNGTHSYNGTNTLKADKNVSLSIEQYEKIHEAYQILSKALKQAGLAPLNSKGEKLEAHVTTXNRQVCANPEKKWV
REYINSLEMSgsgggsggggsggggsggggsggggsggggsggggsQELTTICEQIPSPTLESTPYSLSTTTILANGKAMQGVF
EYYKSVTFVSNCGSHPSTTSKGSPINTQYVFKdnsstsMSTIEERVKKIIGEQLGVKQEEVTNNASFVEDLGADSLDTVEL
VMALEEEFDTEIPDEEAEKITTVQAAIDYINGHQAseqkliseedI
>SEQ ID NO: 28: Mk_{6P4-CCL5}^{c7HopQ} random linkers
(appS4 leader sequence, Megakine Mk_{6P4-CCL5}^{c7HopQ} YD1 depicted in bold, XX is a short peptide linker of
2 AA and random composition, flexible (GGGS)ₙ polypeptide linker, Aga2p protein sequence underlined,
ACP sequence double underlined, cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAQGPPGDIVLACCFAYIARPLPRAHIKEYFYTSGKCSNPAVVFVTXXTTSVIDTTNDAQNLLTQAQTIVN
TLKDYCPILIAKSSSSNGGTNNANTPSWQTAGGGKNSCATFGAEFSAASDMINNAQKIVQETQQLSANQPKNITQ
PHNLNLNSPSSLTALAQKMLKNAQSQAEILKLANQVESDFNKLSSGHLKDYIGKCDASAISSANMTMQNQKNNW
GNGCAGVEETQSLLKTSAADFNNQTPQINQAQNLANTLIQELGNNTYEQLSRLLTNDNGTNSKTSAQAINQAVN
NLNERAKTLAGGTTNSPAYQATLLALRSVLGLWNSMGYAVICGGYTKSPGENNQKDFHYTDENGNGTTINCGGST
NSNGTHSYNGTNTLKADKNVSLSIEQYEKIHEAYQILSKALKQAGLAPLNSKGEKLEAHVTTXXNRQVCANPEKKW
VREYINSLEMSgsgggsggggsggggsggggsggggsggggsggggsQELTTICEQIPSPTLESTPYSLSTTTILANGKAMQG
VFEYYKSVTFVSNCGSHPSTTSKGSPINTQYVFKdnsstsMSTIEERVKKIIGEQLGVKQEEVTNNASFVEDLGADSLDTV
ELVMALEEEFDTEIPDEEAEKITTVQAAIDYINGHQAseqkliseedI
>SEQ ID NO: 29/31: Forward/Reverse Primer for introducing short peptide linker with length 1 amino
acid in the yeast display library of Megakine Mk_{6P4-CCL5}^{c7HopQ}
>SEQ ID NO: 30/32: Forward/Reverse Primer for introducing short peptide linker with length 2 amino
acids in the yeast display library of Megakine Mk_{6P4-CCL5}^{c7HopQ}
>SEQ ID NO: 33: SS-6P4-CCL5
Recombinant soluble 6P4-CCL5 chemokine for production in mammalian cells (HEK293T) (Seq signal
underlined 6P4 sequence (of SEQ ID NO: 1), CCL5)
MKVSAAALAVILIATALCAPASAQGPPGDIVLACCFAYIARPLPRAHIKEYFYTSGKCSNPAVVFVTRKNRQVCANPEK
KWVREYINSLEMS
>SEQ ID NO: 34: Escherichia coli Ygjk protein (PDB 3W7S)
>SEQ ID NO: 35: cYgjk circular permutation linker peptide
>SEQ ID NO: 36: c1YgjK scaffold protein (PDB 3W7S) (YgjK sequences underlined, circular permutation
linker in italics)
KEETQSGLNNYARVVEKGQYDSLEIPAQVAASWESGRDDAAVFGFIDKEQLDKYVANGGKRSDWTVKFAENRSQD
GTLLGYSLLQESVDQASYMYSDNHYLAEMATILGKPEEAKRYRQLAQQLADYINTCMFDPTTQFYYDVRIEDKPLANG
CAGKPIVERGKGPEGWSPLFNGAATQANADAVVKVMLDPKEFNTFVPLGTAALTNPAFGADIYWRGRVWVDQFW
FGLKGMERYGYRDDALKLADTFFRHAKGLTADGPIQENYNPLTGAQQGAPNFSWSAAHLYMLYNDFFRKQasgggs
ggggsggggsgNADNYKNVINRTGAPQYMKDYDYDDHQRFNPFFDLGAWHGHLLPDGPNTMGGFPGVALLTEEYIN
FMASNFDRLTVWQDGKKVDFTLEAYSIPGALVQKLTAKDVQVEMTLRFATPRTSLLETKITSNKPLDLVWDGELLEKL
EAKEGKPLSDKTIAGEYPDYQRKISATRDGLKVTFGKVRATWDLLTSGESEYQVHKSLPVQTEINGNRFTSKAHINGST
TLYTTYSHLLTAQEVSKEQMQIRDILARPAFYLTASQQRWEEYLKKGLTNPDATPEQTRVAVKAIETLNGNWRSPGGA
VKFNTVTPSVTGRWFSGNQTWPWDTWKQAFAMAHFNPDIAKENIRAVFSWQIQPGDSVRPQDVGFVPDLIAWN
LSPERGGDGGNWNERNTKPSLAAWSVMEVYNVTQDKTWVAEMYPKLVAYHDWWLRNRDHNGNGVPEYGATR
DKAHNTESGEMLFTVKK
>SEQ ID NO: 37: c2YgjK scaffold protein (PDB 3W7S) (YgjK sequences underlined, circular permutation
linker in italics)
VQVEMTLRFATPRTSLLETKITSNKPLDLVWDGELLEKLEAKEGKPLSDKTIAGEYPDYQRKISATRDGLKVTFGKVRAT
WDLLTSGESEYQVHKSLPVQTEINGNRFTSKAHINGSTTLYTTYSHLLTAQEVSKEQMQIRDILARPAFYLTASQQRWE
EYLKKGLTNPDATPEQTRVAVKAIETLNGNWRSPGGAVKFNTVTPSVTGRWFSGNQTWPWDTWKQAFAMAHFN
PDIAKENIRAVFSWQIQPGDSVRPQDVGFVPDLIAWNLSPERGGDGGNWNERNTKPSLAAWSVMEVYNVTQDKT
WVAEMYPKLVAYHDWWLRNRDHNGNGVPEYGATRDKAHNTESGEMLFTVKKGDKEETQSGLNNYARVVEKGQY
DSLEIPAQVAASWESGRDDAAVFGFIDKEQLDKYVANGGKRSDWTVKFAENRSQDGTLLGYSLLQESVDQASYMYS
DNHYLAEMATILGKPEEAKRYRQLAQQLADYINTCMFDPTTQFYYDVRIEDKPLANGCAGKPIVERGKGPEGWSPLF
NGAATQANADAVVKVMLDPKEFNTFVPLGTAALTNPAFGADIYWRGRVWVDQFWFGLKGMERYGYRDDALKLA
DTFFRHAKGLTADGPIQENYNPLTGAQQGAPNFSWSAAHLYMLYNDFFRKQasgggsggggsggggsgNADNYKNVI
NRTGAPQYMKDYDYDDHQRFNPFFDLGAWHGHLLPDGPNTMGGFPGVALLTEEYINFMASNFDRLTVWQDGKK
VDFTLEAYSIPGALVQKLTA
>SEQ ID NO: 38: Mk_{6P4-CCL5}^{c1YgjK}V1 Megakine
(N-terminus of 6P4-CCL5-chemokine, GG short peptide linker, c1YgjK scaffold protein sequence
underlined, GG short peptide linker, C-terminus of 6P4-CCL5 chemokine in bold)
QGPPGDIVLACCFAYIARPLPRAHIKEYFYTSGKCSNPAVVFVTRGGKEETQSGLNNYARVVEKGQYDSLEIPAQVAAS
WESGRDDAAVFGFIDKEQLDKYVANGGKRSDWTVKFAENRSQDGTLLGYSLLQESVDQASYMYSDNHYLAEMATIL
GKPEEAKRYRQLAQQLADYINTCMFDPTTQFYYDVRIEDKPLANGCAGKPIVERGKGPEGWSPLFNGAATQANADA
VVKVMLDPKEFNTFVPLGTAALTNPAFGADIYWRGRVWVDQFWFGLKGMERYGYRDDALKLADTFFRHAKGLTAD
GPIQENYNPLTGAQQGAPNFSWSAAHLYMLYNDFFRKQASGGGSGGGGSGGGGSGNADNYKNVINRTGAPQYM
KDYDYDDHQRFNPFFDLGAWHGHLLPDGPNTMGGFPGVALLTEEYINFMASNFDRLTVWQDGKKVDFTLEAYSIP
GALVQKLTAKDVQVEMTLRFATPRTSLLETKITSNKPLDLVWDGELLEKLEAKEGKPLSDKTIAGEYPDYQRKISATRDG
LKVTFGKVRATWDLLTSGESEYQVHKSLPVQTEINGNRFTSKAHINGSTTLYTTYSHLLTAQEVSKEQMQIRDILARPAF
YLTASQQRWEEYLKKGLTNPDATPEQTRVAVKAIETLNGNWRSPGGAVKFNTVTPSVTGRWFSGNQTWPWDTWK
QAFAMAHFNPDIAKENIRAVFSWQIQPGDSVRPQDVGFVPDLIAWNLSPERGGDGGNWNERNTKPSLAAWSVME
VYNVTQDKTWVAEMYPKLVAYHDWWLRNRDHNGNGVPEYGATRDKAHNTESGEMLFTVKKGGNRQVCANPEK
KWVREYINSLEMS
>SEQ ID NO: 39: Mk_{6P4-CCL5}^{c1YgjK}V2 Megakine
(N-terminus of 6P4-CCL5-chemokine, G short peptide linker, c1YgjK scaffold protein sequence
underlined, G short peptide linker, C-terminus of 6P4-CCL5 chemokine in bold)
QGPPGDIVLACCFAYIARPLPRAHIKEYFYTSGKCSNPAVVFVTRGKEETQSGLNNYARVVEKGQYDSLEIPAQVAASW
ESGRDDAAVFGFIDKEQLDKYVANGGKRSDWTVKFAENRSQDGTLLGYSLLQESVDQASYMYSDNHYLAEMATILG
KPEEAKRYRQLAQQLADYINTCMFDPTTQFYYDVRIEDKPLANGCAGKPIVERGKGPEGWSPLFNGAATQANADAV
VKVMLDPKEFNTFVPLGTAALTNPAFGADIYWRGRVWVDQFWFGLKGMERYGYRDDALKLADTFFRHAKGLTADG
PIQENYNPLTGAQQGAPNFSWSAAHLYMLYNDFFRKQASGGGSGGGGSGGGGSGNADNYKNVINRTGAPQYMK
DYDYDDHQRFNPFFDLGAWHGHLLPDGPNTMGGFPGVALLTEEYINFMASNFDRLTVWQDGKKVDFTLEAYSIPG
ALVQKLTAKDVQVEMTLRFATPRTSLLETKITSNKPLDLVWDGELLEKLEAKEGKPLSDKTIAGEYPDYQRKISATRDGL
KVTFGKVRATWDLLTSGESEYQVHKSLPVQTEINGNRFTSKAHINGSTTLYTTYSHLLTAQEVSKEQMQIRDILARPAF
YLTASQQRWEEYLKKGLTNPDATPEQTRVAVKAIETLNGNWRSPGGAVKFNTVTPSVTGRWFSGNQTWPWDTWK
QAFAMAHFNPDIAKENIRAVFSWQIQPGDSVRPQDVGFVPDLIAWNLSPERGGDGGNWNERNTKPSLAAWSVME
VYNVTQDKTWVAEMYPKLVAYHDWWLRNRDHNGNGVPEYGATRDKAHNTESGEMLFTVKKGNRQVCANPEKK
WVREYINSLEMS
>SEQ ID NO: 40: Mk_{6P4-CCL5}^{c1YgjK}V3 Megakine
(N-terminus of 6P4-CCL5-chemokine, c1YgjK scaffold protein sequence underlined, C-terminus of 6P4-
CCL5 chemokine in bold)
QGPPGDIVLACCFAYIARPLPRAHIKEYFYTSGKCSNPAVVFVTRKEETQSGLNNYARVVEKGQYDSLEIPAQVAASWE
SGRDDAAVFGFIDKEQLDKYVANGGKRSDWTVKFAENRSQDGTLLGYSLLQESVDQASYMYSDNHYLAEMATILGK
PEEAKRYRQLAQQLADYINTCMFDPTTQFYYDVRIEDKPLANGCAGKPIVERGKGPEGWSPLFNGAATQANADAVV
KVMLDPKEFNTFVPLGTAALTNPAFGADIYWRGRVWVDQFWFGLKGMERYGYRDDALKLADTFFRHAKGLTADGP
IQENYNPLTGAQQGAPNFSWSAAHLYMLYNDFFRKQASGGGSGGGGSGGGGSGNADNYKNVINRTGAPQYMKD
YDYDDHQRFNPFFDLGAWHGHLLPDGPNTMGGFPGVALLTEEYINFMASNFDRLTVWQDGKKVDFTLEAYSIPGAL
VQKLTAKDVQVEMTLRFATPRTSLLETKITSNKPLDLVWDGELLEKLEAKEGKPLSDKTIAGEYPDYQRKISATRDGLKV
TFGKVRATWDLLTSGESEYQVHKSLPVQTEINGNRFTSKAHINGSTTLYTTYSHLLTAQEVSKEQMQIRDILARPAFYLT
ASQQRWEEYLKKGLTNPDATPEQTRVAVKAIETLNGNWRSPGGAVKFNTVTPSVTGRWFSGNQTWPWDTWKQA
FAMAHFNPDIAKENIRAVFSWQIQPGDSVRPQDVGFVPDLIAWNLSPERGGDGGNWNERNTKPSLAAWSVMEVY
NVTQDKTWVAEMYPKLVAYHDWWLRNRDHNGNGVPEYGATRDKAHNTESGEMLFTVKKNRQVCANPEKKWV
REYINSLEMS
>SEQ ID NO: 41: Mk_{6P4-CCL5}^{c2YgjK}V1 Megakine
(N-terminus of 6P4-CCL5-chemokine, _GG short peptide linker, c2YgjK scaffold protein sequence
underlined, GG short peptide linker, C-terminus of 6P4-CCL5 chemokine in bold)
QGPPGDIVLACCFAYIARPLPRAHIKEYFYTSGKCSNPAVVFVTRGGVQVEMTLRFATPRTSLLETKITSNKPLDLVWD
GELLEKLEAKEGKPLSDKTIAGEYPDYQRKISATRDGLKVTFGKVRATWDLLTSGESEYQVHKSLPVQTEINGNRFTSKA
HINGSTTLYTTYSHLLTAQEVSKEQMQIRDILARPAFYLTASQQRWEEYLKKGLTNPDATPEQTRVAVKAIETLNGNW
RSPGGAVKFNTVTPSVTGRWFSGNQTWPWDTWKQAFAMAHFNPDIAKENIRAVFSWQIQPGDSVRPQDVGFVP
DLIAWNLSPERGGDGGNWNERNTKPSLAAWSVMEVYNVTQDKTWVAEMYPKLVAYHDWWLRNRDHNGNGVP
EYGATRDKAHNTESGEMLFTVKKGDKEETQSGLNNYARVVEKGQYDSLEIPAQVAASWESGRDDAAVFGFIDKEQL
DKYVANGGKRSDWTVKFAENRSQDGTLLGYSLLQESVDQASYMYSDNHYLAEMATILGKPEEAKRYRQLAQQLADY
INTCMFDPTTQFYYDVRIEDKPLANGCAGKPIVERGKGPEGWSPLFNGAATQANADAVVKVMLDPKEFNTFVPLGT
AALTNPAFGADIYWRGRVWVDQFWFGLKGMERYGYRDDALKLADTFFRHAKGLTADGPIQENYNPLTGAQQGAP
NFSWSAAHLYMLYNDFFRKQASGGGSGGGGSGGGGSGNADNYKNVINRTGAPQYMKDYDYDDHQRFNPFFDLG
AWHGHLLPDGPNTMGGFPGVALLTEEYINFMASNFDRLIVWQDGKKVDFTLEAYSIPGALVQKLTAGGNRQVCAN
PEKKWVREYINSLEMS
>SEQ ID NO: 42: Mk_{6P4-CCL5}^{c2YgjK}V3 Megakine
(N-terminus of 6P4-CCL5-chemokine, c2YgjK scaffold protein sequences underlined, C-terminus of 6P4-
CCL5 chemokine in bold)
QGPPGDIVLACCFAYIARPLPRAHIKEYFYTSGKCSNPAVVFVTRVEMTLRFATPRTSLLETKITSNKPLDLVWDGEL
LEKLEAKEGKPLSDKTIAGEYPDYQRKISATRDGLKVTFGKVRATWDLLTSGESEYQVHKSLPVQTEINGNRFTSKAHIN
GSTTLYTTYSHLLTAQEVSKEQMQIRDILARPAFYLTASQQRWEEYLKKGLTNPDATPEQTRVAVKAIETLNGNWRSP
GGAVKFNTVTPSVTGRWFSGNQTWPWDTWKQAFAMAHFNPDIAKENIRAVFSWQIQPGDSVRPQDVGFVPDLIA
WNLSPERGGDGGNWNERNTKPSLAAWSVMEVYNVTQDKTWVAEMYPKLVAYHDWWLRNRDHNGNGVPEYG
ATRDKAHNTESGEMLFTVKKGDKEETQSGLNNYARVVEKGQYDSLEIPAQVAASWESGRDDAAVFGFIDKEQLDKY
VANGGKRSDWTVKFAENRSQDGTLLGYSLLQESVDQASYMYSDNHYLAEMATILGKPEEAKRYRQLAQQLADYINT
CMFDPTTQFYYDVRIEDKPLANGCAGKPIVERGKGPEGWSPLFNGAATQANADAVVKVMLDPKEFNTFVPLGTAAL
TNPAFGADIYWRGRVWVDQFWFGLKGMERYGYRDDALKLADTFFRHAKGLTADGPIQENYNPLTGAQQGAPNFS
WSAAHLYMLYNDFFRKQASGGGSGGGGSGGGGSGNADNYKNVINRTGAPQYMKDYDYDDHQRFNPFFDLGAW
HGHLLPDGPNTMGGFPGVALLTEEYINFMASNFDRLTVWQDGKKVDFTLEAYSIPGALVQKLTANRQVCANPEKK
WVREYINSLEMS
>SEQ ID NO: 43: Mk_{6P4-CCL5}^{c1YgjK}V1_Aga2p_ACP protein sequence
(appS4 leader sequence, Megakine Mk_{6P4-CCL5}^{c1YgjK}V1 depicted in bold, flexible (GGGS)ₙ polypeptide
linker, Aga2p protein sequence underlined, ACP sequence double underlined, cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAQGPPGDIVLACCFAYIARPLPRAHIKEYFYTSGKCSNPAVVFVTRGGKEETQSGLNNYARVVEKGQY
DSLEIPAQVAASWESGRDDAAVFGFIDKEQLDKYVANGGKRSDWTVKFAENRSQDGTLLGYSLLQESVDQASYM
YSDNHYLAEMATILGKPEEAKRYRQLAQQLADYINTCMFDPTTQFYYDVRIEDKPLANGCAGKPIVERGKGPEGW
SPLFNGAATQANADAVVKVMLDPKEFNTFVPLGTAALTNPAFGADIYWRGRVWVDQFWFGLKGMERYGYRDD
ALKLADTFFRHAKGLTADGPIQENYNPLTGAQQGAPNFSWSAAHLYMLYNDFFRKQASGGGSGGGGSGGGGSG
NADNYKNVINRTGAPQYMKDYDYDDHQRFNPFFDLGAWHGHLLPDGPNTMGGFPGVALLTEEYINFMASNFD
RLTVWQDGKKVDFTLEAYSIPGALVQKLTAKDVQVEMTLRFATPRTSLLETKITSNKPLDLVWDGELLEKLEAKEGK
PLSDKTIAGEYPDYQRKISATRDGLKVTFGKVRATWDLLTSGESEYQVHKSLPVQTEINGNRFTSKAHINGSTTLYTT
YSHLLTAQEVSKEQMQIRDILARPAFYLTASQQRWEEYLKKGLTNPDATPEQTRVAVKAIETLNGNWRSPGGAVK
FNTVTPSVTGRWFSGNQTWPWDTWKQAFAMAHFNPDIAKENIRAVFSWQIQPGDSVRPQDVGFVPDLIAWN
LSPERGGDGGNWNERNTKPSLAAWSVMEVYNVTQDKTWVAEMYPKLVAYHDWWLRNRDHNGNGVPEYGA
TRDKAHNTESGEMLFTVKKGGNRQVCANPEKKWVREYINSLEMSIgggsggggsggggsggggsggggsggggsggggs
QELTTICEQIPSPTLESTPYSLSTTTILANGKAMQGVFEYYKSVTFVSNCGSHPSTTSKGSPINTQYVFKdnsstsMSTIEER
VKKIIGEQLGVKQEEVTNNASFVEDLGADSLDTVELVMALEEEFDTEIPDEEAEKITTVQAAIDYINGHQAseqkliseedI
>SEQ ID NO: 44: Mk_{6P4-CCL5}^{c1YgjK}V2_Aga2p_ACP protein sequence
(appS4 leader sequence, Megakine Mk_{6P4-CCL}^{c1YgjK}V2 depicted in bold, flexible (GGGS)ₙ polypeptide
linker, Aga2p protein sequence underlined, ACP sequence double underlined, cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAQGPPGDIVLACCFAYIARPLPRAHIKEYFYTSGKCSNPAVVFVTRGKEETQSGLNNYARVVEKGQYDS
LEIPAQVAASWESGRDDAAVFGFIDKEQLDKYVANGGKRSDWTVKFAENRSQDGTLLGYSLLQESVDQASYMYS
DNHYLAEMATILGKPEEAKRYRQLAQQLADYINTCMFDPTTQFYYDVRIEDKPLANGCAGKPIVERGKGPEGWSP
LFNGAATQANADAVVKVMLDPKEFNTFVPLGTAALTNPAFGADIYWRGRVWVDQFWFGLKGMERYGYRDDAL
KLADTFFRHAKGLTADGPIQENYNPLTGAQQGAPNFSWSAAHLYMLYNDFFRKQASGGGSGGGGSGGGGSGN
ADNYKNVINRTGAPQYMKDYDYDDHQRFNPFFDLGAWHGHLLPDGPNTMGGFPGVALLTEEYINFMASNFDRL
TVWQDGKKVDFTLEAYSIPGALVQKLTAKDVQVEMTLRFATPRTSLLETKITSNKPLDLVWDGELLEKLEAKEGKPL
SDKTIAGEYPDYQRKISATRDGLKVTFGKVRATWDLLTSGESEYQVHKSLPVQTEINGNRFTSKAHINGSTTLYTTYS
HLLTAQEVSKEQMQIRDILARPAFYLTASQQRWEEYLKKGLTNPDATPEQTRVAVKAIETLNGNWRSPGGAVKFN
TVTPSVTGRWFSGNQTWPWDTWKQAFAMAHFNPDIAKENIRAVFSWQIQPGDSVRPQDVGFVPDLIAWNLSP
ERGGDGGNWNERNTKPSLAAWSVMEVYNVTQDKTWVAEMYPKLVAYHDWWLRNRDHNGNGVPEYGATRD
KAHNTESGEMLFTVKKGNRQVCANPEKKWVREYINSLEMSIgggsggggsggggsggggsggggsggggsggggsQELT
TICEQIPSPTLESTPYSLSTTTILANGKAMQGVFEYYKSVTFVSNCGSHPSTTSKGSPINTQYVFKdnsstsMSTIEERVKKII
GEQLGVKQEEVTNNASFVEDLGADSLDTVELVMALEEEFDTEIPDEEAEKITTVQAAIDYINGHQAseqkliseedI
>SEQ ID NO: 45: Mk_{6P4-CCL5}^{c1YgjK}V3_Aga2p_ACP protein sequence
(appS4 leader sequence, Megakine Mk_{6P4-CCL5}^{c1YgjK}V3 depicted in bold, flexible (GGGS)ₙ polypeptide
linker, Aga2p protein sequence underlined, ACP sequence double underlined, cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAQGPPGDIVLACCFAYIARPLPRAHIKEYFYTSGKCSNPAVVFVTRKEETQSGINNYARVVEKGQYDSL
EIPAQVAASWESGRDDAAVFGFIDKEQLDKYVANGGKRSDWTVKFAENRSQDGTLLGYSLLQESVDQASYMYSD
NHYLAEMATILGKPEEAKRYRQLAQQLADYINTCMFDPTTQFYYDVRIEDKPLANGCAGKPIVERGKGPEGWSPLF
NGAATQANADAVVKVMLDPKEFNTFVPLGTAALTNPAFGADIYWRGRVWVDQFWFGLKGMERYGYRDDALKL
ADTFFRHAKGLTADGPIQENYNPLTGAQQGAPNFSWSAAHLYMLYNDFFRKQASGGGSGGGGSGGGGSGNAD
NYKNVINRTGAPQYMKDYDYDDHQRFNPFFDLGAWHGHLLPDGPNTMGGFPGVALLTEEYINFMASNFDRLTV
WQDGKKVDFTLEAYSIPGALVQKLTAKDVQVEMTLRFATPRTSLLETKITSNKPLDLVWDGELLEKLEAKEGKPLSD
KTIAGEYPDYQRKISATRDGLKVTFGKVRATWDLLTSGESEYQVHKSLPVQTEINGNRFTSKAHINGSTTLYTTYSHL
LTAQEVSKEQMQIRDILARPAFYLTASQQRWEEYLKKGLTNPDATPEQTRVAVKAIETLNGNWRSPGGAVKFNTV
TPSVTGRWFSGNQTWPWDTWKQAFAMAHFNPDIAKENIRAVFSWQIQPGDSVRPQDVGFVPDLIAWNLSPER
GGDGGNWNERNTKPSLAAWSVMEVYNVTQDKTWVAEMYPKLVAYHDWWLRNRDHNGNGVPEYGATRDKA
HNTESGEMLFTVKKNRQVCANPEKKWVREYINSLEMSIgggsggggsggggsggggsggggsggggsggggsQELTTICE
QIPSPTLESTPYSLSTTTILANGKAMQGVFEYYKSVTFVSNCGSHPSTTSKGSPINTQYVFKdnsstsMSTIEERVKKIIGEQ
LGVKQEEVTNNASFVEDLGADSLDTVELVMALEEEFDTEIPDEEAEKITTVQAAIDYINGHQAseqkliseedI
>SEQ ID NO: 46: Mk_{6P4-CCL5}^{c2YgjK}V1_Aga2p_ACP protein sequence
(appS4 leader sequence, Megakine Mk_{6P4-CCL5}^{c2YgjK}V1 depicted in bold, flexible (GGGS)ₙ polypeptide
linker, Aga2p protein sequence underlined, ACP sequence double underlined, cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAQGPPGDIVLACCFAYIARPLPRAHIKEYFYTSGKCSNPAVVFVTRGGVQVEMTLRFATPRTSLLETKIT
SNKPLDLVWDGELLEKLEAKEGKPLSDKTIAGEYPDYQRKISATRDGLKVTFGKVRATWDLLTSGESEYQVHKSLPV
QTEINGNRFTSKAHINGSTTLYTTYSHLLTAQEVSKEQMQIRDILARPAFYLTASQQRWEEYLKKGLTNPDATPEQT
RVAVKAIETLNGNWRSPGGAVKFNTVTPSVTGRWFSGNQTWPWDTWKQAFAMAHFNPDIAKENIRAVFSWQI
QPGDSVRPQDVGFVPDLIAWNLSPERGGDGGNWNERNTKPSLAAWSVMEVYNVTQDKTWVAEMYPKLVAYH
DWWLRNRDHNGNGVPEYGATRDKAHNTESGEMLFTVKKGDKEETQSGLNNYARVVEKGQYDSLEIPAQVAAS
WESGRDDAAVFGFIDKEQLDKYVANGGKRSDWTVKFAENRSQDGTLLGYSLLQESVDQASYMYSDNHYLAEMA
TILGKPEEAKRYRQLAQQLADYINTCMFDPTTQFYYDVRIEDKPLANGCAGKPIVERGKGPEGWSPLFNGAATQA
NADAVVKVMLDPKEFNTFVPLGTAALTNPAFGADIYWRGRVWVDQFWFGLKGMERYGYRDDALKLADTFFRH
AKGLTADGPIQENYNPLTGAQQGAPNFSWSAAHLYMLYNDFFRKQASGGGSGGGGSGGGGSGNADNYKNVIN
RTGAPQYMKDYDYDDHQRFNPFFDLGAWHGHLLPDGPNTMGGFPGVALLTEEYINFMASNFDRLTVWQDGKK
VDFTLEAYSIPGALVQKLTAGGNRQVCANPEKKWVREYINSLEMSIgggsggggsggggsggggsggggsggggsggggs
QELTTICEQIPSPTLESTPYSLSTTTILANGKAMQGVFEYYKSVTFVSNCGSHPSTTSKGSPINTQYVFKdnsstsMSTIEER
VKKIIGEQLGVKQEEVTNNASFVEDLGADSLDTVELVMALEEEFDTEIPDEEAEKITTVQAAIDYINGHQAseqkliseedI
>SEQ ID NO: 47: Mk_{6P4-CCL5}^{c2YgjK}V3_Aga2p_ACP protein sequence
(appS4 leader sequence, Megakine Mk_{6P4-CCL5}^{c2YgjK}V3 depicted in bold, flexible (GGGS)ₙ polypeptide
linker, Aga2p protein sequence underlined, ACP sequence double underlined, cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAQGPPGDIVLACCFAYIARPLPRAHIKEYFYTSGKCSNPAVVFVTRVQVEMTLRFATPRTSLLETKITSNK
PLDLVWDGELLEKLEAKEGKPLSDKTIAGEYPDYQRKISATRDGLKVTFGKVRATWDLLTSGESEYQVHKSLPVQTEI
NGNRFTSKAHINGSTTLYTTYSHLLTAQEVSKEQMQIRDILARPAFYLTASQQRWEEYLKKGLTNPDATPEQTRVA
VKAIETLNGNWRSPGGAVKFNTVTPSVTGRWFSGNQTWPWDTWKQAFAMAHFNPDIAKENIRAVFSWQIQP
GDSVRPQDVGFVPDLIAWNLSPERGGDGGNWNERNTKPSLAAWSVMEVYNVTQDKTWVAEMYPKLVAYHD
WWLRNRDHNGNGVPEYGATRDKAHNTESGEMLFTVKKGDKEETQSGLNNYARVVEKGQYDSLEIPAQVAASW
ESGRDDAAVFGFIDKEQLDKYVANGGKRSDWTVKFAENRSQDGTLLGYSLLQESVDQASYMYSDNHYLAEMATI
LGKPEEAKRYRQLAQQLADYINTCMFDPTTQFYYDVRIEDKPLANGCAGKPIVERGKGPEGWSPLFNGAATQANA
DAVVKVMLDPKEFNTFVPLGTAALTNPAFGADIYWRGRVWVDQFWFGLKGMERYGYRDDALKLADTFFRHAK
GLTADGPIQENYNPLTGAQQGAPNFSWSAAHLYMLYNDFFRKQASGGGSGGGGSGGGGSGNADNYKNVINRT
GAPQYMKDYDYDDHQRFNPFFDLGAWHGHLLPDGPNTMGGFPGVALLTEEYINFMASNFDRLTVWQDGKKVD
FTLEAYSIPGALVQKLTANRQVCANPEKKWVREYINSLEMSIgggsggggsggggsggggsggggsggggsggggsQELTT
ICEQIPSPTLESTPYSLSTTTILANGKAMQGVFEYYKSVTFVSNCGSHPSTTSKGSPINTQYVFKdnsstsMSTIEERVKKII
GEQLGVKQEEVTNNASFVEDLGADSLDTVELVMALEEEFDTEIPDEEAEKITTVQAAIDYINGHQAseqkliseedI
>SEQ ID NO: 48: mature form of human IL-1β
>SEQ ID NO: 49: Mk_{IL}_1β^{c7HopQ}V1 Megakine
(N-terminus of IL-1β interleukin, GG short peptide linker, HopQ sequence underlined, GG short peptide
linker, C-terminus of IL-1β interleukin in bold)
APVRSLNCTLRDSQQKSLVMSGPYELKALHLQGQDMEQQVVFSMSFVQGEESNDKIPVALGLKEKNLYLSCVLGGKT
TSVIDTTNDAQNLLTQAQTIVNTLKDYCPILIAKSSSSNGGTNNANTPSWQTAGGGKNSCATFGAEFSAASDMINNA
QKIVQETQQLSANQPKNITQPHNLNLNSPSSLTALAQKMLKNAQSQAEILKLANQVESDFNKLSSGHLKDYIGKCDAS
AISSANMTMQNQKNNWGNGCAGVEETQSLLKTSAADFNNQTPQINQAQNLANTLIQELGNNTYEQLSRLLTNDN
GTNSKTSAQAINQAVNNLNERAKTLAGGTTNSPAYQATLLALRSVLGLWNSMGYAVICGGYTKSPGENNQKDFHYT
DENGNGTTINCGGSTNSNGTHSYNGTNTLKADKNVSLSIEQYEKIHEAYQILSKALKQAGLAPLNSKGEKLEAHVTTSK
GGPTLQLESVDPKNYPKKKMEKRFVFNKIEINNKLEFESAQFPNWYISTSQAENMPVFLGGTKGGQDITDFTMQF
VSS
>SEQ ID NO: 50: Mk_{IL}_1β^{c7HopQ}V2 Megakine
(N-terminus of IL-1β interleukin, G short peptide linker, HopQ sequence underlined, G short peptide
linker, C-terminus of IL-1β interleukin in bold)
APVRSLNCTLRDSQQKSLVMSGPYELKALHLQGQDMEQQVVFSMSFVQGEESNDKIPVALGLKEKNLYLSCVLGKTT
SVIDTTNDAQNLLTQAQTIVNTLKDYCPILIAKSSSSNGGTNNANTPSWQTAGGGKNSCATFGAEFSAASDMINNAQ
KIVQETQQLSANQPKNITQPHNLNLNSPSSLTALAQKMLKNAQSQAEILKLANQVESDFNKLSSGHLKDYIGKCDASAI
SSANMTMQNQKNNWGNGCAGVEETQSLLKTSAADFNNQTPQINQAQNLANTLIQELGNNTYEQLSRLLTNDNGT
NSKTSAQAINQAVNNLNERAKTLAGGTTNSPAYQATLLALRSVLGLWNSMGYAVICGGYTKSPGENNQKDFHYTDE
NGNGTTINCGGSTNSNGTHSYNGTNTLKADKNVSLSIEQYEKIHEAYQILSKALKQAGLAPLNSKGEKLEAHVTTSKGP
TLQLESVDPKNYPKKKMEKRFVFNKIEINNKLEFESAQFPNWYISTSQAENMPVFLGGTKGGQDITDFTMQFVSS
>SEQ ID NO: 51: Mk_{IL}_1β^{c7HopQ}V3 Megakine
(N-terminus of IL-1β interleukin, HopQ sequence underlined, C-terminus of IL-1β interleukin in bold)
APVRSLNCTLRDSQQKSLVMSGPYELKALHLQGQDMEQQVVFSMSFVQGEESNDKIPVALGLKEKNLYLSCVLKTTS
VIDTTNDAQNLLTQAQTIVNTLKDYCPILIAKSSSSNGGTNNANTPSWQTAGGGKNSCATFGAEFSAASDMINNAQK
IVQETQQLSANQPKNITQPHNLNLNSPSSLTALAQKMLKNAQSQAEILKLANQVESDFNKLSSGHLKDYIGKCDASAIS
SANMTMQNQKNNWGNGCAGVEETQSLLKTSAADFNNQTPQINQAQNLANTLIQELGNNTYEQLSRLLTNDNGTN
SKTSAQAINQAVNNLNERAKTLAGGTTNSPAYQATLLALRSVLGLWNSMGYAVICGGYTKSPGENNQKDFHYTDEN
GNGTTINCGGSTNSNGTHSYNGTNTLKADKNVSLSIEQYEKIHEAYQILSKALKQAGLAPLNSKGEKLEAHVTTSKPTL
QLESVDPKNYPKKKMEKRFVFNKIEINNKLEFESAQFPNWYISTSQAENMPVFLGGTKGGQDITDFTMQFVSS
>SEQ ID NO: 52: Mk_{IL}_1β^{c7HopQ}V1_Aga2p_ACP protein sequence
(appS4 leader sequence, Megakine Mk_{IL}_iβ^{c7HopQ}V1 depicted in bold, flexible (GGGS)ₙ polypeptide linker,
Aga2p protein sequence underlined, ACP sequence double underlined, cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAAPVRSLNCTLRDSQQKSLVMSGPYELKALHLQGQDMEQQVVFSMSFVQGEESNDKIPVALGLKEK
NLYLSCVLGGKTTSVIDTTNDAQNLLTQAQTIVNTLKDYCPILIAKSSSSNGGTNNANTPSWQTAGGGKNSCATFG
AEFSAASDMINNAQKIVQETQQLSANQPKNITQPHNLNLNSPSSLTALAQKMLKNAQSQAEILKLANQVESDFNK
LSSGHLKDYIGKCDASAISSANMTMQNQKNNWGNGCAGVEETQSLLKTSAADFNNQTPQINQAQNLANTLIQEL
GNNTYEQLSRLLTNDNGTNSKTSAQAINQAVNNLNERAKTLAGGTTNSPAYQATLLALRSVLGLWNSMGYAVIC
GGYTKSPGENNQKDFHYTDENGNGTTINCGGSTNSNGTHSYNGTNTLKADKNVSLSIEQYEKIHEAYQILSKALKQ
AGLAPLNSKGEKLEAHVTTSKGGPTLQLESVDPKNYPKKKMEKRFVFNKIEINNKLEFESAQFPNWYISTSQAENM
PVFLGGTKGGQDITDFTMQFVSSIgggsggggsggggsggggsggggsggggsggggsQELTTICEQIPSPTLESTPYSLSTT
TILANGKAMQGVFEYYKSVTFVSNCGSHPSTTSKGSPINTQYVFKdnsstsMSTIEERVKKIIGEQLGVKQEEVTNNASF
VEDLGADSLDTVELVMALEEEFDTEIPDEEAEKITTVQAAIDYINGHQAseqkliseedI
>SEQ ID NO: 53: Mk_{IL}_1β^{c7HopQ}V2_Aga2p_ACP protein sequence
(appS4 leader sequence, Megakine Mk_{IL}_1β^{c7HopQ}V2 depicted in bold, flexible (GGGS)ₙ polypeptide linker,
Aga2p protein sequence underlined, ACP sequence double underlined, cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAAPVRSLNCTLRDSQQKSLVMSGPYELKALHLQGQDMEQQVVFSMSFVQGEESNDKIPVALGLKEK
NLYLSCVLGKTTSVIDTTNDAQNLLTQAQTIVNTLKDYCPILIAKSSSSNGGTNNANTPSWQTAGGGKNSCATFGA
EFSAASDMINNAQKIVQETQQLSANQPKNITQPHNLNLNSPSSLTALAQKMLKNAQSQAEILKLANQVESDFNKL
SSGHLKDYIGKCDASAISSANMTMQNQKNNWGNGCAGVEETQSLLKTSAADFNNQTPQINQAQNLANTLIQEL
GNNTYEQLSRLLTNDNGTNSKTSAQAINQAVNNLNERAKTLAGGTTNSPAYQATLLALRSVLGLWNSMGYAVIC
GGYTKSPGENNQKDFHYTDENGNGTTINCGGSTNSNGTHSYNGTNTLKADKNVSLSIEQYEKIHEAYQILSKALKQ
AGLAPLNSKGEKLEAHVTTSKGPTLQLESVDPKNYPKKKMEKRFVFNKIEINNKLEFESAQFPNWYISTSQAENMP
VFLGGTKGGQDITDFTMQFVSSIgggsggggsggggsggggsggggsggggsggggsQELTTICEQIPSPTLESTPYSLSTTTI
LANGKAMQGVFEYYKSVTFVSNCGSHPSTTSKGSPINTQYVFKdnsstsMSTIEERVKKIIGEQLGVKQEEVTNNASFVE
DLGADSLDTVELVMALEEEFDTEIPDEEAEKITTVQAAIDYINGHQAseqkliseedI
>SEQ ID NO: 54: Mk_{IL}_1β^{c7HopQ}V3_Aga2p_ACP protein sequence
(appS4 leader sequence, Megakine Mk_{IL}_1β^{c7HopQ}V3 depicted in bold, flexible (GGGS)ₙ polypeptide linker,
Aga2p protein sequence underlined, ACP sequence double underlined, cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAAPVRSLNCTLRDSQQKSLVMSGPYELKALHLQGQDMEQQVVFSMSFVQGEESNDKIPVALGLKEK
NLYLSCVLKTTSVIDTTNDAQNLLTQAQTIVNTLKDYCPILIAKSSSSNGGTNNANTPSWQTAGGGKNSCATFGAEF
SAASDMINNAQKIVQETQQLSANQPKNITQPHNLNLNSPSSLTALAQKMLKNAQSQAEILKLANQVESDFNKLSS
GHLKDYIGKCDASAISSANMTMQNQKNNWGNGCAGVEETQSLLKTSAADFNNQTPQINQAQNLANTLIQELGN
NTYEQLSRLLTNDNGTNSKTSAQAINQAVNNLNERAKTLAGGTTNSPAYQATLLALRSVLGLWNSMGYAVICGGY
TKSPGENNQKDFHYTDENGNGTTINCGGSTNSNGTHSYNGTNTLKADKNVSLSIEQYEKIHEAYQILSKALKQAGL
APLNSKGEKLEAHVTTSKPTLQLESVDPKNYPKKKMEKRFVFNKIEINNKLEFESAQFPNWYISTSQAENMPVFLGG
TKGGQDITDFTMQFVSSIgggsggggsggggsggggsggggsggggsggggsQELTTICEQIPSPTLESTPYSLSTTTILANGK
AMQGVFEYYKSVTFVSNCGSHPSTTSKGSPINTQYVFKdnsstsMSTIEERVKKIIGEQLGVKQEEVTNNASFVEDLGAD
SLDTVELVMALEEEFDTEIPDEEAEKITTVQAAIDYINGHQAseqkliseedI
>SEQ ID NO: 55: IL-1β_Aga2p_ACP protein sequence
(appS4 leader sequence, IL-1β depicted in bold, flexible (GGGS)ₙ polypeptide linker, Aga2p protein
sequence underlined, ACP sequence double underlined, cMyc Tag)
MRFPSIFTAVVFAASSALAAPANTTAEDETAQIPAEAVIGYLGLEGDSDVAALPLSDSTNNGSLSTNTTIASIAAKEEGV
QLDKREAEAAPVRSLNCTLRDSQQKSLVMSGPYELKALHLQGQDMEQQVVFSMSFVQGEESNDKIPVALGLKEK
NLYLSCVLKDDKPTLQLESVDPKNYPKKKMEKRFVFNKIEINNKLEFESAQFPNWYISTSQAENMPVFLGGTKGGQ
DITDFTMQFVSSIgggsggggsggggsggggsggggsggggsggggsQELTTICEQIPSPTLESTPYSLSTTTILANGKAMQG
VFEYYKSVTFVSNCGSHPSTTSKGSPINTQYVFKdnsstsMSTIEERVKKIIGEQLGVKQEEVTNNASFVEDLGADSLDTV
ELVMALEEEFDTEIPDEEAEKITTVQAAIDYINGHQAseqkliseedI

### REFERENCES

Bliven, S., Prlic, A. (2012). Circular permutation in proteins. PLOS Comput. Biol. 8(3):e1002445.

Boder, E. T., and Wittrup, K. D. (1997). Yeast surface display for screening combinatorial polypeptide libraries. Nat Biotechnol 15, 553-557.

Chao, G., Lau, W. L., Hackel, B. J., Sazinsky, S. L., Lippow, S. M., and Wittrup, K. D. (2006). Isolating and engineering human antibodies using yeast surface display. Nat Protoc 1, 755-768.

Dixon A S et al. 2016. NanoLuc Complementation Reporter Optimized for Accurate Measurement of Protein Interactions in Cells. ACS Chem Biol, 11(2):400-8

Gustaysson M. et al., 2017. Structural basis of ligand interaction with atypical chemokine receptor 3. Nature Comm. 8:14135.

Javaheri, A., Kruse, T., Moonens, K., Mejías-Luque, R., Debraekeleer, A., Asche, C. I., Tegtmeyer, N., Kalali, B., Bach, N. C., Sieber, S. A., Hill, D. J., Königer, V., Hauck, C. R., Moskalenko, R., Haas, R., Busch, D. H., Klaile, E., Slevogt, H., Schmidt, A., Backert, S., Remaut, H., Singer, B. B., and Gerhard, M. (2016). Helicobacter pylori adhesin HopQ engages in a virulence-enhancing interaction with human CEACAMs. Nature Microbiology 2, 16189.

Johnsson, N., George, N., and Johnsson, K. (2005). Protein chemistry on the surface of living cells. Chembiochem: a European journal of chemical biology 6, 47-52.

King I. C., Gleixner, J., Doyle, L., Kuzin, A., Hunt, J. F., Xiao, R., Montelione, G. T., Stoddard, B. L., DiMaio, F., and Baker, D. (2015). Precise assembly of complex beta sheet topologies from de novo designed building blocks. eLife 4:e11012. doi: 10.7554/eLife.11012.

Koide, S. (2009). Engineering of recombinant crystallization chaperones. Curr Opin Struct Biol 19(4): 449-457.

Kufareva I. et al., 2015. Chemokine and chemokine receptor structure and interactions: implications for therapeutic strategies. Immunol Cell Biol. 93(4): 372-383.

Kurakata, Y. Uechi, A. Yoshida, H. Kamitori, S. Sakano, Y. Nishikawa, A. Tonozuka, T. (2008). Structural Insights into the Substrate Specificity and Function of Escherichia coli K12 YgjK, a Glucosidase Belonging to the Glycoside Hydrolase Family 63. J. Mol. Biol. 381, 116-128.

Manglik, A., Kobilka, B. K., and Steyaert, J. (2017). Nanobodies to Study G Protein-Coupled Receptor Structure and Function. Annu Rev Pharmacol Toxicol. 57: 19-37.

Martin A C. (2000). The ups and downs of protein topology; rapid comparison of protein structure. Protein Eng. 13(12):829-37.

Nogales, E. (2016). The development of cryo-EM into a mainstream structural biology technique. Nature Methods 13, 24-27.

Orengo et al. (1994). Protein superfamilies and domain superfolds. Nature. 15;372(6507):631-4.

Pardon, E., Laeremans, T., Triest, S., Rasmussen, S. G., Wohlkonig, A., Ruf, A., Muyldermans, S., Hol, W. G., Kobilka, B. K., and Steyaert, J. (2014). A general protocol for the generation of Nanobodies for structural biology. Nature Protocols. 9: 674-693.

Proudfoot A. E. I. et al. 2015. Targeting chemokines: Pathogens can, why can't we? Cytokine 74 (2015) 259-267

Rakestraw J, Sazinsky S, Piatesi A, Antipov E, Wittrup K. (2009). Directed evolution of a secretory leader for the improved expression of heterologous proteins and full-length antibodies in Saccharomyces cerevisiae. Biotechnol. Bioeng. 103, 1192-1201.

Ramesh, G. et al. Cytokines and Chemokines at the crossroads of neuroinflammation, neurodegeneration, and neuropathic pain. Hinawi Publishing Group, Mediators of Inflammation ID480739, (2013)

Wan, Q. et al. (2018) Mini G protein probes for active G protein-coupled receptors (GPCRs) in live cells. J Biol Chem 293,7466-7473.

Wang, D. Zhang, S. Li, L. Liu, X. Mei, K. Wang, X. (2010). Structural insights into the assembly and activation of IL-1β with its receptors. Nature Immunology, 11, 905-911.

Zheng et al. (2017) Structure of CC Chemokine Receptor 5 with a Potent Chemokine Antagonist Reveals Mechanisms of Chemokine Recognition and Molecular Mimicry by HIV. Immunity, 46: 1005-1017.

1. A functional fusion protein comprising: a cytokine fused with a scaffold protein,
wherein the scaffold protein is a folded protein of at least 50 amino acids that interrupts the topology of the cytokine at one or more accessible sites in an exposed β-turn of a β-strand-containing domain of the cytokine via two or more fusions, wherein the fusions are direct fusions or fusions made by a linker.

2. The functional fusion protein of claim 1, wherein the cytokine is a chemokine and wherein the scaffold protein interrupts the topology of a core domain of the chemokine at one or more accessible sites in an exposed β-turn of the core domain.

3. The functional fusion protein of claim 2, wherein the chemokine core domain comprises a N-terminal loop, a β-sheet comprising 3 β-strands, and a C-terminal helix, and wherein the scaffold protein is inserted in the exposed β-turn that connects β-strand β2 and β-strand β3 of the chemokine core domain.

4. The functional fusion protein of claim 1, wherein the cytokine is an interleukin and wherein the scaffold protein interrupts the topology of the interleukin β-barrel core motif at one or more accessible sites in an exposed β-turn of the β-barrel core motif.

5. The functional fusion protein of claim 4, wherein the interleukin is an IL-1 family interleukin.

6. The functional fusion protein of claim 1, wherein the scaffold protein is a circularly permutated protein.

7. The functional fusion protein of claim 1, wherein the scaffold protein has a total molecular mass of at least 30 kDa.

8. A nucleic acid molecule encoding the fusion protein of claim 1.

9. The nucleic acid molecule of claim 8, wherein the nucleic acid is comprised in a vector.

10. The nucleic acid molecule of claim 9, wherein the vector is optimized for expression in E. coli, for surface display in yeast, in phages, in bacteria, or in viruses.

11. The fusion protein of claim 1, wherein the fusion protein is comprised in a host cell.

12. The fusion protein of claim 11, wherein said the fusion protein and a cytokine receptor are co-expressed in the host cell.

13. The fusion protein of claim 1, wherein the fusion protein is present in a complex comprising
(i) the fusion protein, and
(ii) a receptor protein,
wherein the receptor protein is bound to the cytokine of the fusion protein.

14. The fusion protein of claim 13, wherein the receptor is activated upon binding to the fusion protein.

15. A method for determining a 3-dimensional structure of a ligand/receptor complex comprising the steps of:
(i) providing the fusion protein of claim 13;
(ii) displaying the complex in suitable conditions for structural analysis,
wherein the 3D structure of the ligand/receptor complex is determined at high-resolution.

16. (canceled)

17. The method according to claim 16, wherein determining the 3D structure of the ligand/receptor complex comprises single particle cryo-EM or crystallography.

18. A method for producing the fusion protein of claim 3, the method comprising:
(i) selecting a chemokine, and a scaffold protein with accessible β-turns for interruption of the chemokine protein sequence without interruption of chemokine core domain topology;
(ii) designing a genetic fusion construct encoding:
a) the protein sequence of the chemokine interrupted between the β-strand β2 and β-strand β3 of the core domain,
b) the scaffold protein, wherein the N- and C-terminal ends of the scaffold protein are fused to obtain a circularly permutated scaffold protein,
wherein the circularly permutated scaffold protein of b) is interrupted in its amino acid sequence at an accessible site, loop, or turn, different from the original N- or C-terminus, and,
c) the amino acid at the interrupted site of the chemokine C-terminally of β-strand β2 fused to the amino acid of the most N-terminally interrupted site of the circularly permutated scaffold protein, and the amino acid of the interrupted site of the chemokine N-terminally of β-strand β3 fused to the amino acid most C-terminally of the interrupted site of the circularly permutated scaffold protein;
(iii) introducing the gene fusion construct into an expression system to obtain a fusion protein wherein the chemokine is fused at two sites of its core domain to the circularly permutated scaffold protein.
